(19) United States
(12) Patent Application Publication
(10) Pub. No.: US 2015/0344444 A1
(43) Pub. Date: Dec. 3, 2015

(54) BET-PROTEIN-INHIBITING DIHYDROXYQUINOXALINONES

(71) Applicant: BAYER PHARMA AKTIENGESELLSCHAFT, Berlin (DE)

(72) Inventors: Norbert SCHMEES, Berlin (DE); Bernard HAENDLER, Berlin (DE); Detlef STÖCKIGT, Potsdam (DE); Daniel GALLENKAMP, Wuppertal (DE)

(21) Appl. No.: 14/654,576

(22) PCT Filed: Dec. 17, 2013
(86) PCT No.: PCT/EP2013/076785

§ 371 Date: Jun. 22, 2015

(30) Foreign Application Priority Data
Dec. 20, 2012 (EP) 12198623.6
Oct. 8, 2013 (EP) 13187778.9
Oct. 18, 2013 (EP) 13189263.0

(51) Int. Cl. C07D 241/44; C07D 405/12; C07D 495/10; A61K 31/5377; C07D 403/12; C07D 405/04; A61K 31/498; C07D 401/12; C07D 491/107

(52) U.S. Cl. CPC C07D 241/44; C07D 401/12; C07D 405/12; C07D 495/10; C07D 491/107; C07D 403/12; C07D 405/04; A61K 31/498; A61K 31/5377

(57) ABSTRACT

The present invention relates to BET protein-inhibitory, especially BRD4-inhibitory, dihydroquinoxalinones of the general formula (I)

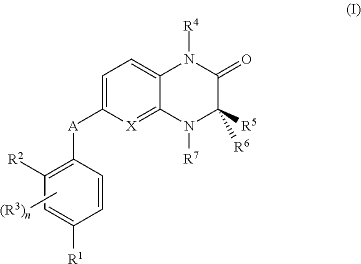

in which A, X, R¹, R², R³, R⁴, R⁵, R⁶, R⁷ and n are each as defined in the description, to intermediates for preparation of the inventive compounds, to pharmaceutical compositions comprising the inventive compounds, and to the prophylactic and therapeutic use thereof in the case of hyperproliferative disorders, especially in the case of neoplastic disorders. This invention further relates to the use of BET protein inhibitors in viral infections, in neurodegenerative disorders, in inflammation disorders, in atherosclerotic disorders and in male fertility control.

The present invention relates to BET protein-inhibitory, especially BRD4-inhibitory, dihydroquinoxalinones, to intermediates for preparation of the inventive compounds, to pharmaceutical compositions comprising the inventive compounds, and to the prophylactic and therapeutic use thereof in the case of hyperproliferative disorders, especially in the case of neoplastic disorders. This invention further relates to the use of BET protein inhibitors in viral infections, in neurodegenerative disorders, in inflammation diseases, in atherosclerotic disorders and in male fertility control.

The human BET family (bromo domain and extra C-terminal domain family) has four members (BRD2, BRD3, BRD4 and BRDT) containing two related bromo domains and one extraterminal domain (Wu and Chiang, J. Biol. Chem., 2007, 282:13141-13145). The bromo domains are protein regions which recognize acetylated lysine residues. Such acetylated lysines are often found at the N-terminal end of histones (e.g. histone H3 or histone H4) and are features of an open chromatin structure and active gene transcription (Kuo and Allis, Bioessays, 1998, 20:615-626). In addition, bromo domains may recognize further acetylated proteins. For example, BRD4 binds to RelA, which leads to stimulation of NF-κB and transcriptional activity of inflammatory genes (Huang et al., Mol. Cell. Biol., 2009, 29:1375-1387). BRD4 also binds to cyclin T1 and forms an active complex which is important for transcription elongation (Schröder et al., J. Biol. Chem., 2012, 287:1090-1099). The extraterminal domain of BRD2, BRD3 and BRD4 interacts with several proteins involved in chromatin modulation and the regulation of gene expression (Rahman et al., Mol. Cell. Biol., 2011, 31:2641-2652).

In mechanistic terms, BET proteins play an important role in cell growth and in the cell cycle. They are associated with mitotic chromosomes, which suggests a role in epigenetic memory (Dey et al., Mol. Biol. Cell, 2009, 20:4899-4909; Yang et al., Mol. Cell. Biol., 2008, 28:967-976). Involvement of BRD4 in the post-mitotic reactivation of gene transcription has been demonstrated (Zhao et al., Nat. Cell. Biol., 2011, 13:1295-1304). BRD4 is essential for transcription elongation and recruits the elongation complex P-TEFb consisting of CDK9 and cyclin T1, which leads to activation of RNA polymerase II (Yang et al., Mol. Cell, 2005, 19:535-545; Schröder et al., J. Biol. Chem., 2012, 287:1090-1099). Consequently, the expression of genes involved in cell proliferation is stimulated, for example of c-Myc, cyclin D1 and aurora B (You et al., Mol. Cell. Biol., 2009, 29:5094-5103; Zuber et al., Nature, 2011, doi:10.1038). BRD2 is involved in the regulation of target genes of the androgen receptor (Draker et al., PLOS Genetics, 2012, 8, e1003047). BRD2 and BRD3 bind to transcribed genes in hyperacetylated chromatin regions and promote transcription by RNA polymerase II (LeRoy et al., Mol. Cell, 2008, 30:51-60).

The knockdown of BRD4 or the inhibition of the interaction with acetylated histones in various cell lines leads to a G1 arrest (Mochizuki et al., J. Biol. Chem., 2008, 283:9040-9048; Mertz et al., Proc. Natl. Acad. Sci. USA, 2011, 108:16669-16674). It has also been shown that BRD4 binds to promoter regions of several genes which are activated in the G1 phase, for example cyclin D1 and D2 (Mochizuki et al., J. Biol. Chem., 2008, 283:9040-9048). In addition, inhibition of the expression of c-Myc, an essential factor in cell proliferation, after BRD4 inhibition has been demonstrated (Dawson et al., Nature, 2011, 478:529-533; Delmore et al., Cell, 2011, 146:1-14; Mertz et al., Proc. Natl. Acad. Sci. USA, 2011, 108:16669-16674) Inhibition of the expression of androgen-regulated genes and binding of BRD2 to corresponding regulatory regions has also been demonstrated (Draker et al., PLOS Genetics, 2012, 8, e1003047).

BRD2 and BRD4 knockout mice die at an early stage during embryogenesis (Gyuris et al., Biochim Biophys. Acta, 2009, 1789:413-421; Houzelstein et al., Mol. Cell. Biol., 2002, 22:3794-3802). Heterozygotic BRD4 mice have various growth defects attributable to reduced cell proliferation (Houzelstein et al., Mol. Cell. Biol., 2002, 22:3794-3802).

BET proteins play an important role in various tumour types. Fusion between the BET proteins BRD3 or BRD4 and NUT, a protein which is normally expressed only in the testes, leads to an aggressive form of squamous cell carcinoma, called NUT midline carcinoma (French, Cancer Genet. Cytogenet., 2010, 203:16-20). The fusion protein prevents cell differentiation and promotes proliferation (Yan et al., J. Biol. Chem., 2011, 286:27663-27675). The growth of in vivo models derived therefrom is inhibited by a BRD4 inhibitor (Filippakopoulos et al., Nature, 2010, 468:1067-1073). Screening for therapeutic targets in an acute myeloid leukaemia cell line (AML) showed that BRD4 plays an important role in this tumour (Zuber et al., Nature, 2011, 478, 524-528). Reduction in BRD4 expression leads to a selective arrest of the cell cycle and to apoptosis. Treatment with a BRD4 inhibitor prevents the proliferation of an AML xenograft in vivo. Further experiments with a BRD4 inhibitor show that BRD4 is involved in various haematological tumours, for example multiple myeloma (Delmore et al., Cell, 2011, 146, 904-917) and Burkitt's lymphoma (Mertz et al., Proc. Natl. Acad. Sci. USA, 2011, 108, 16669-16674). In solid tumours too, for example lung cancer, BRD4 plays an important role (Lockwood et al., Proc. Natl. Acad. Sci. USA, 2012, 109, 19408-19413). Elevated expression of BRD4 has been detected in multiple myeloma, and amplification of the BRD4 gene has also been found in patients having multiple myeloma (Delmore et al., Cell, 2011, 146, 904-917). Amplification of the DNA region containing the BRD4 gene was detected in primary breast tumours (Kadota et al., Cancer Res, 2009, 69:7357-7365). For BRD2 too, there are data relating to a role in tumours. A transgenic mouse which overexpresses BRD2 selectively in B cells develops B cell lymphoma and leukaemia (Greenwall et al., Blood, 2005, 103:1475-1484).

BET proteins are also involved in viral infections. BRD4 binds to the E2 protein of various papillomaviruses and is important for the survival of the viruses in latently infected cells (Wu et al., Genes Dev., 2006, 20:2383-2396; Vosa et al., J. Virol., 2006, 80:8909-8919). The herpes virus, which is responsible for Kaposi's sarcoma, also interacts with various BET proteins, which is important for disease survival (Viejo-Borbolla et al., J. Virol., 2005, 79:13618-13629; You et al., J. Virol., 2006, 80:8909-8919). Through binding to P-TEFb, BRD4 also plays an important role in the replication of HIV-1 (Bisgrove et al., Proc. Natl Acad. Sci. USA, 2007, 104:13690-13695).

Treatment with a BRD4 inhibitor leads to stimulation of the dormant, untreatable reservoir of HIV-1 viruses in T cells (Banerjee et al., J. Leukoc. Biol., 2012, 92, 1147-1154). This reactivation could enable new therapeutic methods for AIDS treatment (Zinchenko et al., J. Leukoc. Biol., 2012, 92, 1127-1129). A critical role of BRD4 in DNA replication of polyomaviruses has also been reported (Wang et al., PLoS Pathog., 2012, 8, doi:10.1371).

BET proteins are additionally involved in inflammation processes. BRD2-hypomorphic mice show reduced inflammation in adipose tissue (Wang et al., Biochem. J., 2009, 425:71-83). Infiltration of macrophages in white adipose tissue is also reduced in BRD2-deficient mice (Wang et al., Biochem. J., 2009, 425:71-83). It has also been shown that BRD4 regulates a number of genes involved in inflammation. In LPS-stimulated macrophages, a BRD4 inhibitor prevents the expression of inflammatory genes, for example IL-1 or IL-6 (Nicodeme et al., Nature, 2010, 468:1119-1123).

BET proteins are also involved in the regulation of the ApoA1 gene (Mirguet et al., Bioorg. Med. Chem. Lett., 2012, 22:2963-2967). The corresponding protein is part of high-density lipoprotein (HDL), which plays an important role in atherosclerosis (Smith, Arterioscler. Thromb. Vasc. Biol., 2010, 30:151-155). Through the stimulation of ApoA1 expression, BET protein inhibitors can increase the concentrations of cholesterol HDL and hence may potentially be useful for the treatment of atherosclerosis (Mirguet et al., Bioorg. Med. Chem. Lett., 2012, 22:2963-2967).

The BET protein BRDT plays an essential role in spermatogenesis through the regulation of the expression of several genes important during and after meiosis (Shang et al., Development, 2007, 134:3507-3515; Matzuk et al., Cell, 2012, 150:673-684). In addition, BRDT is involved in the post-meiotic organization of chromatin (Dhar et al., J. Biol. Chem., 2012, 287:6387-6405). In vivo experiments in mice show that treatment with a BET inhibitor which also inhibits BRDT leads to a decrease in sperm production and infertility (Matzuk et al., Cell, 2012, 150:673-684).

All these studies show that the BET proteins play an essential role in various pathologies, and also in male fertility. It would therefore be desirable to find potent and selective inhibitors which prevent the interaction between the BET proteins and acetylated proteins. These novel inhibitors should also have suitable pharmacokinetic properties which allow inhibition of these interactions in vivo, i.e. in patients.

It has now been found that substituted dihydroquinoxalines have the desired properties, i.e. show BRD4-inhibitory action. The inventive compounds are thus valuable active ingredients for prophylactic and therapeutic use in the case of hyperproliferative disorders, especially in the case of neoplastic disorders. In addition, the inventive compounds can be employed in the case of viral infections, in the case of neurodegenerative disorders, in the case of inflammation disorders, in the case of atherosclerotic disorders and in male fertility control.

### PRIOR ART

The nomenclature applied in the assessment of the prior art (derived from the nomenclature software ACD Name batch, Version 12.01, from Advanced Chemical Development, Inc.) is illustrated by the following diagrams:

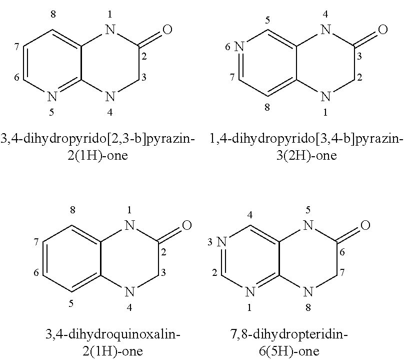

Based on the chemical structure, only very few types of BRD4 inhibitors have been described to date (Chun-Wa Chung et al., Progress in Medicinal Chemistry 2012, 51, 1-55).

The first published BRD4 inhibitors were diazepines. For example, phenylthienotriazolo-1,4-diazepines (4-phenyl-6H-thieno[3,2-f][1,2,4]triazolo[4,3-a][1,4]diazepines) are described in WO2009/084693 (Mitsubishi Tanabe Pharma Corporation) and as compound JQ1 in WO2011/143669 (Dana Farber Cancer Institute). The replacement of the thieno unit with a benzo unit likewise leads to active inhibitors (J. Med. Chem. 2011, 54, 3827-3838; E. Nicodeme et al., Nature 2010, 468, 1119). Further 4-phenyl-6H-thieno[3,2-f][1,2,4]triazolo[4,3-a][1,4]diazepines and related compounds having alternative rings as a fusion partner rather than the benzo unit are claimed generically or described explicitly in WO2012/075456 (Constellation Pharmaceuticals).

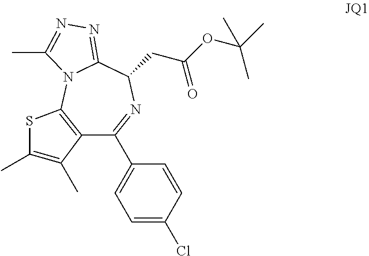 [SMILES: Cc1sc2c(c1C)C(c1ccc(Cl)cc1)=N[C@@H](CC(=O)OC(C)(C)C)c1nnc(C)n1-2]

Azepines as BRD4 inhibitors have recently been described in WO2012/075383 (Constellation Pharmaceuticals). This application relates to 6-substituted 4H-isoxazolo[5,4-d][2]benzazepines and 4H-isoxazolo[3,4-d][2]benzazepines, including those compounds which have optionally substituted phenyl at position 6, and also to analogues with alternative heterocyclic fusion partners rather than the benzo unit, for example thieno- or pyridoazepines. Another structural class of BRD4 inhibitors described is that of 7-isoxazoloquinolines and related quinolone derivatives (Bioorganic & Medicinal Chemistry Letters 22 (2012) 2963-2967). WO2011/054845 (GlaxoSmithKline) describes further benzodiazepines as BRD4 inhibitors.

The inventive compounds, in contrast, are substituted 3,4-dihydroquinoxalin-2(1H)-one derivatives which differ structurally in various ways from the above-discussed chemotypes of BRD4 inhibitors. Because of the significant structural differences, it could not have been assumed that the compounds claimed here also have BRD4-inhibitory action. It is therefore surprising that the inventive compounds have good inhibitory action in spite of the considerable structural differences.

Some 3,4-dihydroquinoxalin-2(1H)-one derivatives substituted at C-6 by an aromatic amino group, in which the phenyl group is in turn substituted by a para-amide group (corresponding to 2-oxo-1,2,3,4-tetrahydroquinoxaline derivatives), are indexed by Chemical Abstracts as “Chemical Library” substances without a literature reference [see 4-{[(3R)-4-cyclopentyl-3-ethyl-1-methyl-2-oxo-1,2,3,4-tetrahydroquinoxalin-6-yl]amino}-3-methoxy-N-[2-methyl-1-(pyrrolidin-1-yl)propan-2-yl]benzamide, CAS Registry No. 1026451-60-4, N-(1-benzylpiperidin-4-yl)-4-{[(3R)-4-cyclopentyl-1,3-dimethyl-2-oxo-1,2,3,4-tetrahydroquinoxalin-6-yl]amino}-3-methoxybenzamide, CAS Registry No. 1026961-36-3, 4-{[(3R)-4-cyclohexyl-1,3-dimethyl-2-oxo-1,2,3,4-tetrahydroquinoxalin-6-yl]amino}-N-[1-(dimethylamino)-2-methylpropan-2-yl]-3-methoxybenzamide, CAS Registry No. 1025882-57-8]. No therapeutic use for these compounds has been described to date.

Some documents include compounds which are structurally similar but are aimed at completely different mechanisms of action, and in some cases also other indications. Dihydroquinoxalinones and related bicyclic systems have been described in a series of patent applications.

US 2006/0019961 (P. E. Mahaney et al.) describes substituted 3,4-dihydroquinoxalin-2(1H)-one derivatives as modulators of the oestrogen receptor for treatment of various inflammation disorders, cardiovascular disorders and autoimmune disorders. The example substances disclosed in this application have only small substituents (such as halogen or methyl) at C-6, but a substituent which necessarily has a hydroxylated aromatic system at N-4, by virtue of which the substances differ from the compounds of this present invention.

WO 2008/117061 describes a series of bicyclic chemotypes, including 3,4-dihydroquinoxalin-2(1H)-one derivatives, as inhibitors of steroid sulphatase, for uses including inhibition of the growth of tumours. The substances claimed in the application mentioned differ from the substances disclosed in this present invention, for example, by the substitution at N-1. In the case of this present invention, this is restricted to small alkyl groups, preferably methyl, whereas the substitution at N-1 in WO 2008/117061 must necessarily contain an aromatic R³ group.

WO 2006/050064, WO 2007/134169 and US 2009/0264384 (Nuada LLC) describe a series of bicyclic chemotypes, including 3,4-dihydroquinoxalin-2(1H)-one derivatives, as inhibitors of various isoforms of phosphodiesterase for treatment of inflammation disorders among others. N-1 in the structures claimed is substituted by a group characterized by a carboxamide or a terminal group derived from the boronic acid, which differ from the compounds of this present invention.

WO 2012/088314 (Agios Pharmaceuticals) discloses a series of bicyclic chemotypes, including dihydroquinoxalinones, as modulators of pyruvate kinase M2. The substances described therein differ from the compounds of this present invention, for example, by the -D-Q-D¹- moiety, the totality of which cannot represent an A group of the present invention (—NH— or —O—).

U.S. Pat. No. 6,369,057 (EP 0509398; Aventis Pharma) describes various quinoxaline and quinoxalinone derivatives as active antiviral ingredients. The substances disclosed therein differ from the compounds of this present invention by the type and position of the substituents. EP 0657166 and EP 0728481 describe combinations of such compounds with nucleosides or protease inhibitors having antiviral action.

WO 2007/022638 (Methylgene Inc.) discloses, in quite general terms, HDAC inhibitors of several chemotypes, but the structures of the example compounds disclosed differ distinctly from the compounds of the present invention.

WO 1999/050254 (Pfizer) describes a series of bicyclic chemotypes as inhibitors of serine proteases for antithrombotic therapy, but these compounds differ distinctly by the type and position of the substituents from the inventive compounds.

WO 2010/085570 (Takeda Pharmaceutical Company) describes inhibitors of poly-ADP-ribose polymerase (PARP) which are derived from a series of bi- and tricyclic skeletons, and which include 3,4-dihydropyrido[2,3-b]pyrazin-2(1H)-one derivatives, as medicaments for treatment of various diseases.

WO 2006/005510 (Boehringer Ingelheim) describes 1,4-dihydropyrido[3,4-b]pyrazin-3(2H)-one derivatives as inhibitors of PLK-1 for treatment of hyperproliferative disorders.

It has now been found that compounds of the general formula (I)

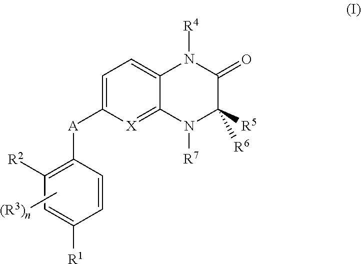

in which
A is —NH— or —O—,
X is —CH—,
n is 0 or 1,
R¹ is a —C(═O)NR⁸R⁹ or —S(═O)₂NR⁸R⁹ group,
R² is hydrogen, halogen, cyano, C₁-C₄-alkyl, C₂-C₄-alkenyl, C₂-C₄-alkynyl, halo-C₁-C₄-alkyl, C₁-C₄-alkoxy, halo-C₁-C₄-alkoxy, C₁-C₄-alkylthio, halo-C₁-C₄-alkylthio, or —NR¹⁰R¹¹,
R³ is halogen, C₁-C₃-alkyl, C₁-C₃-alkoxy, trifluoromethyl or cyano and may be bonded to any of the still-unoccupied positions in the aromatic system,
R⁴ is methyl or ethyl,
R⁵ is hydrogen or C₁-C₃-alkyl,
R⁶ is hydrogen or C₁-C₃-alkyl, or
R⁵ and R⁶ together are C₂-C₅-alkylene,
R⁷ is C₁-C₆-alkyl, C₃-C₈-cycloalkyl, 4- to 8-membered heterocycloalkyl, phenyl or phenyl-C₁-C₃-alkyl,
   in which each phenyl radical may optionally be mono-, di- or trisubstituted identically or differently by halogen, cyano, C₁-C₄-alkyl, C₂-C₄-alkenyl, C₂-C₄-alkynyl, C₁-C₄-alkoxy, halo-C₁-C₄-alkyl or halo-C₁-C₄-alkoxy,
R⁸ is C₁-C₆-alkyl which may optionally be mono-, di- or trisubstituted identically or differently by hydroxyl, oxo, fluorine, cyano, C₁-C₄-alkoxy, halo-C₁-C₄-alkoxy, —NR¹⁰R¹¹, 4- to 8-membered heterocycloalkyl, 4- to 8-membered heterocycloalkenyl, C₅-C₁₁-heterospirocycloalkyl, bridged C₆-C₁₂-heterocycloalkyl, C₆-C₁₂-heterobicycloalkyl, phenyl or 5- to 6-membered heteroaryl, in which 4- to 8-membered heterocycloalkyl, 4- to 8-membered heterocycloalkenyl, C₅-C₁₁-heterospirocycloalkyl, bridged C₆-C₁₂-heterocycloalkyl, C₆-C₁₂-heterobicycloalkyl may optionally be monosubstituted by oxo or C₁-C₃-alkyl,
   and in which phenyl and 5- to 6-membered heteroaryl may optionally be mono- or disubstituted identically or differently by halogen, cyano, trifluoromethyl, C₁-C₃-alkyl or C₁-C₃-alkoxy,
   or is C₃-C₆-alkenyl or C₃-C₆-alkynyl,
   or is C₃-C₈-cycloalkyl or C₄-C₈-cycloalkenyl, which may optionally be mono- or disubstituted identically or differently by hydroxyl, oxo, cyano, fluorine, C₁-C₃-alkyl, C₁-C₃-alkoxy, trifluoromethyl or —NR¹⁰R¹¹,
   or is 4- to 8-membered heterocycloalkyl, 4- to 8-membered heterocycloalkenyl, C₅-C₁₁-heterospirocycloalkyl, bridged C₆-C₁₂-heterocycloalkyl, or C₆-C₁₂-heterobicycloalkyl, which may optionally be mono- or disubstituted identically or differently by hydroxyl, oxo, cyano, fluorine, C₁-C₃-alkyl, C₁-C₃-alkoxy, trifluoromethyl, C₁-C₃-alkylcarbonyl or —NR¹⁰R¹¹,
R⁹ is hydrogen or C₁-C₃-alkyl, or
R⁸ and R⁹, together with the nitrogen atom to which they are bonded, are 4- to 8-membered heterocycloalkyl, 4- to 8-membered heterocycloalkenyl, C₅-C₁₁-heterospirocycloalkyl, bridged C₆-C₁₂-heterocycloalkyl or C₆-C₁₂-heterobicycloalkyl, which may optionally be mono- or disubstituted identically or differently by hydroxyl, oxo, cyano, fluorine, C₁-C₃-alkyl, C₁-C₃-alkoxy, trifluoromethyl or —NR¹⁰R¹¹,
R¹⁰ and R¹¹ are each independently hydrogen or C₁-C₃-alkyl optionally mono- or disubstituted identically or differently by hydroxyl, oxo or fluorine, or
R¹⁰ and R¹¹, together with the nitrogen atom to which they are bonded, are 4- to 8-membered heterocycloalkyl which may optionally be mono- or disubstituted identically or differently by hydroxyl, oxo, cyano, fluorine, C₃-C₆-cycloalkyl-C₁-C₃-alkyl or C₁-C₃-alkyl,
R¹² is C₁-C₆-alkyl or phenyl-C₁-C₃-alkyl,

and the diastereomers, racemates, polymorphs and physiologically acceptable salts thereof, excluding the compounds
4-{[(3R)-4-cyclopentyl-3-ethyl-1-methyl-2-oxo-1,2,3,4-tetrahydroquinoxalin-6-yl]amino}-3-methoxy-N-[2-methyl-1-(pyrrolidin-1-yl)propan-2-yl]benzamide and
4-{[(3R)-4-cyclohexyl-1,3-dimethyl-2-oxo-1,2,3,4-tetrahydroquinoxalin-6-yl]amino}-N-[1-(dimethylamino)-2-methylpropan-2-yl]-3-methoxybenzamide,

surprisingly prevent the interaction between BRD4 and an acetylated histone H4 peptide and hence inhibit the growth of cancer and tumour cells.

Preference is given to those compounds of the general formula (I) in which
A is —NH— or —O—,
X is —CH—,
n is 0 or 1,
R¹ is a —C(═O)NR⁸R⁹ or —S(═O)₂NR⁸R⁹ group,
R² is hydrogen, fluorine, chlorine, cyano, C₁-C₃-alkyl, fluoro-C₁-C₃-alkyl, C₁-C₃-alkoxy, fluoro-C₁-C₃-alkoxy, C₁-C₃-alkylthio or fluoro-C₁-C₃-alkylthio,
R³ is fluorine, chlorine or cyano and may be bonded to any of the still-unoccupied positions in the aromatic system,
R⁴ is methyl or ethyl,
R⁵ is C₁-C₃-alkyl,
R⁶ is hydrogen,
R⁷ is C₂-C₅-alkyl, C₃-C₇-cycloalkyl, 4- to 7-membered heterocycloalkyl or phenyl-C₁-C₃-alkyl,
   in which the phenyl radical may optionally be mono- or disubstituted identically or differently by fluorine, chlorine, bromine, cyano, C₁-C₃-alkyl, C₁-C₃-alkoxy, or trifluoromethyl,
R⁸ is C₁-C₆-alkyl which may optionally be mono-, di- or trisubstituted identically or differently by hydroxyl, oxo, fluorine, cyano, C₁-C₃-alkoxy, fluoro-C₁-C₃-alkoxy, —NR¹⁰R¹¹, 4- to 8-membered heterocycloalkyl, phenyl or 5- to 6-membered heteroaryl,
   in which the 4- to 8-membered heterocycloalkyl may optionally be monosubstituted by oxo or C₁-C₃-alkyl,
   or is C₃-C₈-cycloalkyl which may optionally be mono- or disubstituted identically or differently by hydroxyl, oxo, cyano, fluorine or —NR¹⁰R¹¹,
   or is 4- to 8-membered heterocycloalkyl, C₆-C₈-heterospirocycloalkyl, bridged C₆-C₁₀-heterocycloalkyl or C₆-C₁₀-heterobicycloalkyl, which may optionally be mono- or disubstituted identically or differently by hydroxyl, oxo, cyano, fluorine, C₁-C₃-alkyl, C₁-C₃-alkylcarbonyl or —NR¹⁰R¹¹,
R⁹ is hydrogen or C₁-C₃-alkyl, or
R⁸ and R⁹, together with the nitrogen atom to which they are bonded, are 4- to 8-membered heterocycloalkyl, C₆-C₈-heterospirocycloalkyl, bridged C₆-C₁₀-heterocycloalkyl or C₆-C₁₀-heterobicycloalkyl, which may optionally be mono- or disubstituted identically or differently by hydroxyl, oxo or C₁-C₃-alkyl,
R¹⁰ and R¹¹ are each independently hydrogen or optionally mono-hydroxyl-, -oxo- or -fluorine-substituted C₁-C₃-alkyl, or
R¹⁰ and R¹¹, together with the nitrogen atom to which they are bonded, are 4- to 7-membered heterocycloalkyl which may optionally be mono- or disubstituted identically or differently by hydroxyl, cyano, fluorine, cyclopropylmethyl or C₁-C₃-alkyl,

and the diastereomers, racemates, polymorphs and physiologically acceptable salts thereof, excluding the compounds
4-{[(3R)-4-cyclopentyl-3-ethyl-1-methyl-2-oxo-1,2,3,4-tetrahydroquinoxalin-6-yl]amino}-3-methoxy-N-[2-methyl-1-(pyrrolidin-1-yl)propan-2-yl]benzamide and
4-{[(3R)-4-cyclohexyl-1,3-dimethyl-2-oxo-1,2,3,4-tetrahydroquinoxalin-6-yl]amino}-N-[1-(dimethylamino)-2-methylpropan-2-yl]-3-methoxybenzamide.

Particular preference is given to those compounds of the general formula (I) in which
A is —NH— or —O—,
X is —CH—,
n is 0,
R¹ is a —C(═O)NR⁸R⁹ or —S(═O)₂NR⁸R⁹ group,
R² is hydrogen, fluorine, chlorine, cyano, C₁-C₃-alkyl, fluoro-C₁-C₃-alkyl, C₁-C₃-alkoxy, fluoro-C₁-C₃-alkoxy, C₁-C₃-alkylthio or fluoro-C₁-C₃-alkylthio,
R⁴ is methyl,
R⁵ is methyl or ethyl,
R⁶ is hydrogen,
R⁷ is C₃-C₅-alkyl, C₃-C₇-cycloalkyl, 4- to 7-membered heterocycloalkyl or phenyl-C₁-C₃-alkyl,
   in which the phenyl radical may optionally be mono- or disubstituted identically or differently by fluorine, C₁-C₃-alkyl or C₁-C₃-alkoxy,
R⁸ is C₁-C₄-alkyl which may optionally be monosubstituted by
   —NR¹⁰R¹¹ or 4- to 8-membered heterocycloalkyl,
   in which the 4- to 8-membered heterocycloalkyl may optionally be monosubstituted by oxo or C₁-C₃-alkyl,
   or is C₃-C₈-cycloalkyl which may optionally be monosubstituted by oxo or —NR¹⁰R¹¹,
   or is 4- to 8-membered heterocycloalkyl which may optionally be monosubstituted by oxo, C₁-C₃-alkyl or C₁-C₃-alkylcarbonyl,
R⁹ is hydrogen or methyl, or
R⁸ and R⁹, together with the nitrogen atom to which they are bonded, are 4- to 8-membered heterocycloalkyl or C₆-C₈-heterospirocycloalkyl, which may optionally be mono- or disubstituted identically or differently by oxo or C₁-C₃-alkyl,
R¹⁰ and R¹¹ are each independently hydrogen, methyl or ethyl, or
R¹⁰ and R¹¹, together with the nitrogen atom to which they are bonded, are 4- to 7-membered heterocycloalkyl which may optionally be mono- or disubstituted identically or differently by fluorine, cyclopropylmethyl or C₁-C₃-alkyl,

and the diastereomers, racemates, polymorphs and physiologically acceptable salts thereof, excluding the compounds
4-{[(3R)-4-cyclopentyl-3-ethyl-1-methyl-2-oxo-1,2,3,4-tetrahydroquinoxalin-6-yl]amino}-3-methoxy-N-[2-methyl-1-(pyrrolidin-1-yl)propan-2-yl]benzamide and
4-{[(3R)-4-cyclohexyl-1,3-dimethyl-2-oxo-1,2,3,4-tetrahydroquinoxalin-6-yl]amino}-N-[1-(dimethylamino)-2-methylpropan-2-yl]-3-methoxybenzamide.

Especially preferred are those compounds of the general formula (I) in which
A is —NH— or —O—,
X is —CH—,
n is 0,
R¹ is a —C(═O)NR⁸R⁹ or —S(═O)₂NR⁸R⁹ group,
R² is hydrogen or methoxy,
R⁴ is methyl,
R⁵ is methyl,
R⁶ is hydrogen,
R⁷ is iso-propyl, C₅-C₇-cycloalkyl, 5- or 6-membered heterocycloalkyl or benzyl, in which the phenyl radical present in benzyl may optionally be mono- or disubstituted identically or differently by fluorine or methoxy,
R⁸ is C₁-C₂-alkyl which may optionally be monosubstituted by oxetanyl, pyrrolidinyl, piperidinyl, morpholinyl or piperazinyl,
   in which piperazinyl may optionally be monosubstituted by C₁-C₃-alkyl, or is C₃-C₆-cycloalkyl which may optionally be monosubstituted by oxo or —NR¹⁰R¹¹,
   or is 4- to 6-membered heterocycloalkyl which may optionally be monosubstituted by oxo, methyl or acetyl,
R⁹ is hydrogen or methyl, or
R⁸ and R⁹, together with the nitrogen atom to which they are bonded, are 5- or 6-membered heterocycloalkyl or C₆-C₈-heterospirocycloalkyl, which may optionally be mono- or disubstituted identically or differently by oxo or C₁-C₃-alkyl,
R¹⁰ and R¹¹ are each independently hydrogen, methyl or ethyl, or
R¹⁰ and R¹¹, together with the nitrogen atom to which they are bonded, are pyrrolidinyl, piperidinyl, morpholinyl or piperazinyl bonded via the common nitrogen, where the piperazinyl may optionally be monosubstituted by cyclopropylmethyl or C₁-C₃-alkyl,

and the diastereomers, racemates, polymorphs and physiologically acceptable salts thereof.

Exceptionally preferred are those compounds of the general formula (I) in which
A is —NH— or —O—,
X is —CH—,
n is 0,
R¹ is a —C(═O)NR⁸R⁹ or —S(═O)₂NR⁸R⁹ group,
R² is hydrogen or methoxy,
R⁴ is methyl,
R⁵ is methyl,
R⁶ is hydrogen,
R⁷ is cyclopentyl, cycloheptyl, tetrahydropyran-4-yl, benzyl, 4-methoxybenzyl or 2,6-difluorobenzyl,
R⁸ is one of the following groups:

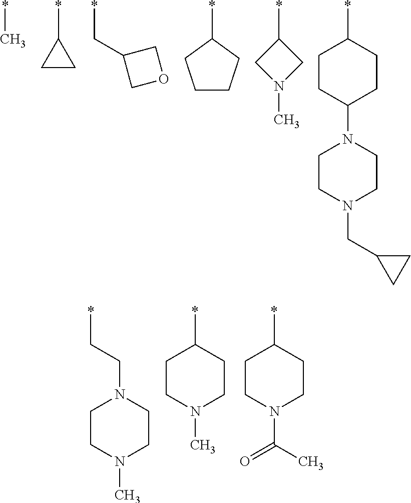

R⁹ is hydrogen or methyl, or
R⁸ and R⁹ together with the nitrogen atom to which they are bonded are one of the following groups:

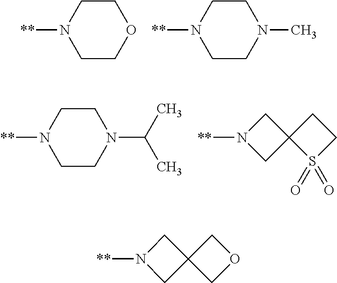

and the diastereomers, racemates, polymorphs and physiologically acceptable salts thereof.

Also exceptionally preferred are those compounds of the general formula (I) in which
A is —NH—,
X is —CH—,
n is 0,
R¹ is a —C(═O)NR⁸R⁹ or —S(═O)₂NR⁸R⁹ group,
R² is hydrogen or methoxy,
R⁴ is methyl,
R⁵ is methyl,
R⁶ is hydrogen,
R⁷ is cyclopentyl, cycloheptyl, tetrahydropyran-4-yl, benzyl, 4-methoxybenzyl or 2,6-difluorobenzyl,
R⁸ is one of the following groups:

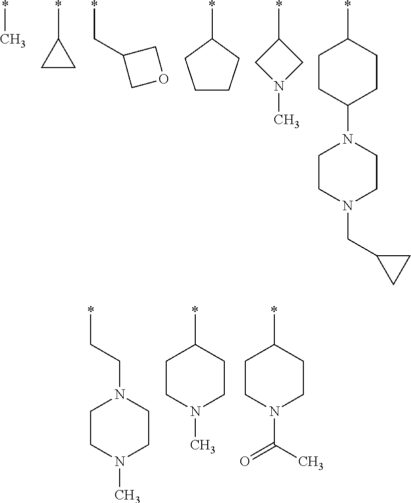

R⁹ is hydrogen or methyl, or
R⁸ and R⁹ together with the nitrogen atom to which they are bonded are one of the following groups:

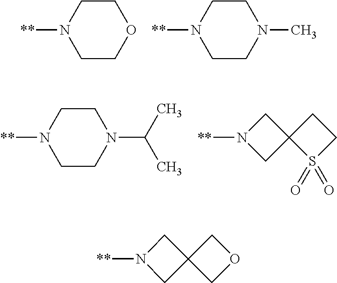

and the diastereomers, racemates, polymorphs and physiologically acceptable salts thereof.

Also exceptionally preferred are those compounds of the general formula (I) in which
A is —O—,
X is —CH—,
n is 0,
R¹ is a —C(═O)NR⁸R⁹ or —S(═O)₂NR⁸R⁹ group,
R² is hydrogen or methoxy,
R⁴ is methyl,
R⁵ is methyl,
R⁶ is hydrogen,
R⁷ is cyclopentyl, cycloheptyl, tetrahydropyran-4-yl, benzyl, 4-methoxybenzyl or 2,6-difluorobenzyl,
R⁸ is one of the following groups:

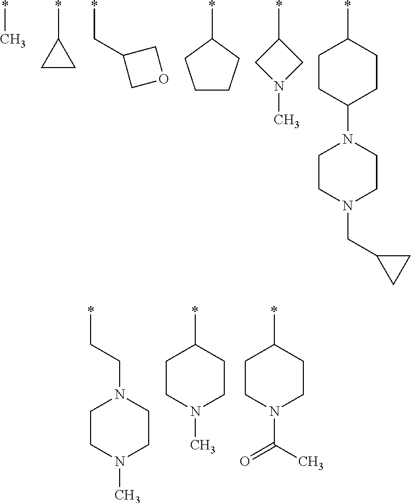

R⁹ is hydrogen or methyl, or
R⁸ and R⁹ together with the nitrogen atom to which they are bonded are one of the following groups:

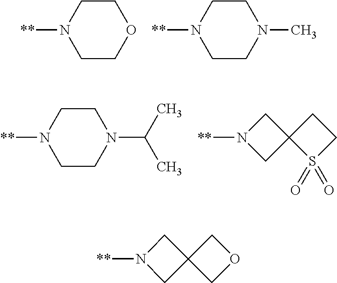

and the diastereomers, racemates, polymorphs and physiologically acceptable salts thereof.

In the definitions, “*” indicates the connection point to the nitrogen atom in —C(═O)NR⁸R⁹ or —S(═O)₂NR⁸R⁹.

In the definitions, “**” indicates the connection point to the carbonyl or sulphonyl group present in R¹.

Preference is additionally given to compounds of the general formula (I) in which A is —NH—.

Preference is given to compounds of the general formula (I) in which A is —O—.

Preference is given to compounds of the general formula (I) in which R¹ is —C(═O)NR⁸R⁹.

Preference is given to compounds of the general formula (I) in which R¹ is —S(═O)₂NR⁸R⁹.

Preference is given to compounds of the general formula (I) in which n is the number 0.

Preference is given to compounds of the general formula (I) in which R² is C₁-C₃-alkoxy.

Preference is given to compounds of the general formula (I) in which R² is ethoxy.

Preference is given to compounds of the general formula (I) in which R² is fluorine.

Preference is given to compounds of the general formula (I) in which R² is chlorine.

Particular preference is given to compounds of the general formula (I) in which R² is methoxy.

Particular preference is given to compounds of the general formula (I) in which R² is hydrogen.

Preference is given to compounds of the general formula (I) in which R⁴ is methyl or ethyl.

Preference is given to compounds of the general formula (I) in which R⁴ is ethyl.

Particular preference is given to compounds of the general formula (I) in which R⁴ is methyl.

Preference is given to compounds of the general formula (I) in which R⁵ is methyl or ethyl.

Preference is given to compounds of the general formula (I) in which R⁵ is ethyl.

Particular preference is given to compounds of the general formula (I) in which R⁵ is methyl.

Preference is given to compounds of the general formula (I) in which R⁶ is hydrogen.

Preference is given to compounds of the general formula (I) in which R⁷ is C₃-C₅-alkyl, C₃-C₇-cycloalkyl, 4- to 7-membered heterocycloalkyl or phenyl-C₁-C₃-alkyl in which the phenyl radical may optionally be mono- or disubstituted identically or differently by fluorine, C₁-C₃-alkyl or C₁-C₃-alkoxy.

Preference is given to compounds of the general formula (I) in which R⁷ is C₃-C₅-alkyl, C₃-C₇-cycloalkyl, 4- to 7-membered heterocycloalkyl or phenyl-C₁-C₃-alkyl.

Preference is given to compounds of the general formula (I) in which R⁷ is C₃-C₅-alkyl.

Preference is given to compounds of the general formula (I) in which R⁷ is C₃-C₆-cycloalkyl.

Preference is given to compounds of the general formula (I) in which R⁷ is phenyl-C₁-C₃-alkyl in which the phenyl radical may optionally be mono- or disubstituted identically or differently by fluorine, C₁-C₃-alkyl or C₁-C₃-alkoxy.

Preference is given to compounds of the general formula (I) in which R⁷ is phenyl-C₁-C₃-alkyl.

Particular preference is given to compounds of the general formula (I) in which R⁷ is iso-propyl, C₅-C₇-cycloalkyl, 5- or 6-membered heterocycloalkyl or benzyl, in which the phenyl radical present in benzyl may optionally be mono- or disubstituted identically or differently by fluorine or methoxy.

Particular preference is given to compounds of the general formula (I) in which R⁷ is iso-propyl.

Particular preference is given to compounds of the general formula (I) in which R⁷ is C₅-C₇-cycloalkyl.

Particular preference is given to compounds of the general formula (I) in which R⁷ is 5- or 6-membered heterocycloalkyl.

Particular preference is given to compounds of the general formula (I) in which R⁷ is benzyl, in which the phenyl radical present in benzyl may optionally be mono- or disubstituted identically or differently by fluorine or methoxy.

Particular preference is given to compounds of the general formula (I) in which R⁷ is cyclopentyl, cycloheptyl, tetrahydropyran-4-yl, benzyl, 4-methoxybenzyl or 2,6-difluorobenzyl.

Particular preference is given to compounds of the general formula (I) in which R⁷ is cyclopentyl or cycloheptyl.

Particular preference is given to compounds of the general formula (I) in which R⁷ is cyclopentyl.

Particular preference is given to compounds of the general formula (I) in which R⁷ is cycloheptyl.

Particular preference is given to compounds of the general formula (I) in which R⁷ is tetrahydropyran-4-yl.

Particular preference is given to compounds of the general formula (I) in which R⁷ is benzyl, 4-methoxybenzyl or 2,6-difluorobenzyl.

Particular preference is given to compounds of the general formula (I) in which R⁷ is 4-methoxybenzyl or 2,6-difluorobenzyl.

Particular preference is given to compounds of the general formula (I) in which R⁷ is benzyl or 4-methoxybenzyl.

Particular preference is given to compounds of the general formula (I) in which R⁷ is benzyl or 2,6-difluorobenzyl.

Particular preference is given to compounds of the general formula (I) in which R⁷ is benzyl.

Particular preference is given to compounds of the general formula (I) in which R⁷ is 4-methoxybenzyl.

Particular preference is given to compounds of the general formula (I) in which R⁷ is 2,6-difluorobenzyl.

Preference is given to compounds of the general formula (I) in which R⁸ is C₁-C₄-alkyl which may optionally be monosubstituted by —NR¹⁰R¹¹ or 4- to 8-membered heterocycloalkyl, in which the 4- to 8-membered heterocycloalkyl may optionally be monosubstituted by oxo or C₁-C₃-alkyl, or is C₃-C₈-cycloalkyl which may optionally be monosubstituted by oxo or —NR¹⁰R¹¹ or is 4- to 8-membered heterocycloalkyl which may optionally be monosubstituted by oxo, C₁-C₃-alkyl or C₁-C₃-alkylcarbonyl.

Preference is given to compounds of the general formula (I) in which R⁸ is C₁-C₄-alkyl which may optionally be monosubstituted by —NR¹⁰R¹¹ or 4- to 8-membered heterocycloalkyl, in which the 4- to 8-membered heterocycloalkyl may optionally be monosubstituted by oxo or C₁-C₃-alkyl.

Preference is given to compounds of the general formula (I) in which R⁸ is C₃-C₈-cycloalkyl which may optionally be monosubstituted by oxo or —NR¹⁰R¹¹.

Preference is given to compounds of the general formula (I) in which R⁸ is 4- to 8-membered heterocycloalkyl which may optionally be monosubstituted by oxo, C₁-C₃-alkyl or C₁-C₃-alkylcarbonyl.

Preference is given to compounds of the general formula (I) in which R⁸ is C₁-C₂-alkyl which may optionally be monosubstituted by oxetanyl, pyrrolidinyl, piperidinyl, morpholinyl or piperazinyl, in which piperazinyl may optionally be monosubstituted by C₁-C₃-alkyl,

or is C₃-C₆-cycloalkyl which may optionally be monosubstituted by oxo or —NR¹⁰R¹¹, or is 4- to 6-membered heterocycloalkyl which may optionally be monosubstituted by oxo, methyl or acetyl.

Preference is given to compounds of the general formula (I) in which R⁸ is C₁-C₂-alkyl which may optionally be monosubstituted by oxetanyl, pyrrolidinyl, piperidinyl, morpholinyl or piperazinyl, in which piperazinyl may optionally be monosubstituted by C₁-C₃-alkyl.

Preference is given to compounds of the general formula (I) in which R⁸ is C₃-C₆-cycloalkyl which may optionally be monosubstituted by oxo or —NR¹⁰R¹¹.

Preference is given to compounds of the general formula (I) in which R⁸ is 4- to 6-membered heterocycloalkyl which may optionally be monosubstituted by oxo, methyl or acetyl.

Particular preference is given to compounds of the general formula (I) in which R⁸ is one of the following groups:

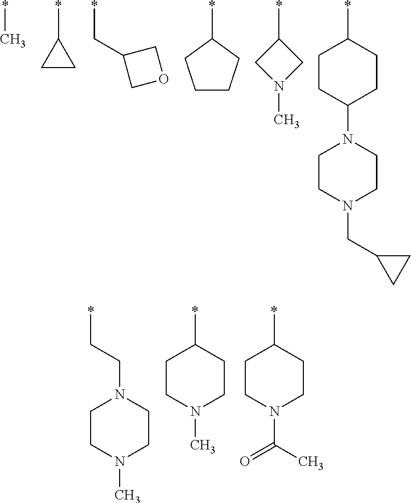

where “*” indicates the connection point to the nitrogen atom in

—C(═O)NR⁸R⁹ or —S(═O)₂NR⁸R⁹.

Preference is given to compounds of the general formula (I) in which R⁹ is hydrogen or C₁-C₃-alkyl.

Preference is given to compounds of the general formula (I) in which R⁹ is hydrogen or methyl.

Preference is given to compounds of the general formula (I) in which R⁹ is hydrogen.

Preference is given to compounds of the general formula (I) in which R⁹ is methyl.

Preference is given to compounds of the general formula (I) in which R⁸ and R⁹, together with the nitrogen atom to which they are bonded, are 4- to 8-membered heterocycloalkyl, C₆-C₈-heterospirocycloalkyl, bridged C₆-C₁₀-heterocycloalkyl or C₆-C₁₀-heterobicycloalkyl, which may optionally be mono- or disubstituted identically or differently by hydroxyl, oxo or C₁-C₃-alkyl.

Preference is given to compounds of the general formula (I) in which R⁸ and R⁹, together with the nitrogen atom to which they are bonded, are 4- to 8-membered heterocycloalkyl which may optionally be mono- or disubstituted identically or differently by hydroxyl, oxo or C₁-C₃-alkyl.

Preference is given to compounds of the general formula (I) in which R⁸ and R⁹, together with the nitrogen atom to which they are bonded, are 4- to 8-membered heterocycloalkyl or C₆-C₈-heterospirocycloalkyl, which may optionally be mono- or disubstituted identically or differently by oxo or C₁-C₃-alkyl.

Preference is given to compounds of the general formula (I) in which R⁸ and R⁹, together with the nitrogen atom to which they are bonded, are 4- to 8-membered heterocycloalkyl which may optionally be mono- or disubstituted identically or differently by oxo or C₁-C₃-alkyl.

Preference is given to compounds of the general formula (I) in which R⁸ and R⁹, together with the nitrogen atom to which they are bonded, are C₆-C₈-heterospirocycloalkyl, which may optionally be mono- or disubstituted identically or differently by oxo or C₁-C₃-alkyl.

Preference is given to compounds of the general formula (I) in which R⁸ and R⁹, together with the nitrogen atom to which they are bonded, are 5- or 6-membered heterocycloalkyl or C₆-C₈-heterospirocycloalkyl, which may optionally be mono- or disubstituted identically or differently by oxo or C₁-C₃-alkyl.

Particular preference is given to compounds of the general formula (I) in which R⁸ and R⁹, together with the nitrogen atom to which they are bonded, are one of the following groups:

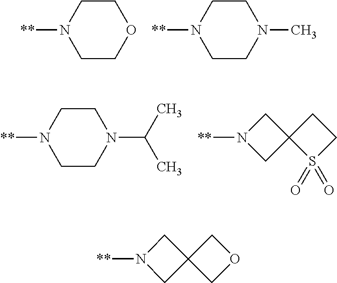

in which “**” indicates the connection point to the carbonyl or sulphonyl group present in R¹.

Preference is given to compounds of the general formula (I) in which R¹⁰ and R¹¹ are each independently hydrogen or optionally mono-hydroxyl-, -oxo- or -fluorine-substituted C₁-C₃-alkyl, or, together with the nitrogen atom to which they are bonded, are 4- to 7-membered heterocycloalkyl which may optionally be mono- or disubstituted identically or differently by hydroxyl, cyano, fluorine, cyclopropylmethyl or C₁-C₃-alkyl.

Preference is given to compounds of the general formula (I) in which R¹⁰ and R¹¹ are each independently hydrogen or optionally mono-hydroxyl-, -oxo- or -fluorine-substituted C₁-C₃-alkyl.

Preference is given to compounds of the general formula (I) in which R¹⁰ and R¹¹, together with the nitrogen atom to which they are bonded, are 4- to 7-membered heterocycloalkyl which may optionally be mono- or disubstituted identically or differently by hydroxyl, cyano, fluorine, cyclopropylmethyl or C₁-C₃-alkyl.

Particular preference is given to compounds of the general formula (I) in which R¹⁰ and R¹¹ are each independently hydrogen, methyl or ethyl, or, together with the nitrogen atom to which they are bonded, are 4- to 7-membered heterocycloalkyl which may optionally be mono- or disubstituted identically or differently by fluorine, cyclopropylmethyl or C₁-C₃-alkyl.

Particular preference is given to compounds of the general formula (I) in which R¹⁰ and R¹¹ are each independently hydrogen, methyl or ethyl.

Particular preference is given to compounds of the general formula (I) in which R¹⁰ and R¹¹, together with the nitrogen atom to which they are bonded, are 4- to 7-membered heterocycloalkyl which may optionally be mono- or disubstituted identically or differently by fluorine, cyclopropylmethyl or C₁-C₃-alkyl.

Particular preference is given to compounds of the general formula (I) in which R¹⁰ and R¹¹ are each independently hydrogen, methyl or ethyl, or, together with the nitrogen atom to which they are bonded, are pyrrolidinyl, piperidinyl, morpholinyl or piperazinyl bonded via the common nitrogen, where the piperazinyl may optionally be monosubstituted by cyclopropylmethyl or C₁-C₃-alkyl.

Particular preference is given to compounds of the general formula (I) in which R¹⁰ and R¹¹, together with the nitrogen atom to which they are bonded, are pyrrolidinyl, piperidinyl, morpholinyl or piperazinyl bonded via the common nitrogen, where the piperazinyl may optionally be monosubstituted by cyclopropylmethyl or C₁-C₃-alkyl.

Particular preference is given to compounds of the general formula (I) in which R¹⁰ is hydrogen, methyl or ethyl.

Particular preference is given to compounds of the general formula (I) in which R¹¹ is hydrogen, methyl or ethyl.

Particular preference is given to compounds of the general formula (I) in which R¹⁰ and R¹¹, together with the nitrogen atom to which they are bonded, are N-cyclopropylmethylpiperazinyl bonded via the common nitrogen.

The specific radical definitions given in the particular combinations or preferred combinations of radicals are, irrespective of the particular combinations of radicals specified, also replaced as desired by radical definitions of other combinations.

Very particular preference is given to combinations of two or more of the abovementioned preferred ranges.

Very particular preference is given to the following compounds of the general formula (I):
N-cyclopentyl-4-{[(3R)-4-cyclopentyl-1,3-dimethyl-2-oxo-1,2,3,4-tetrahydroquinoxalin-6-yl]amino}benzamide;
4-{[(3R)-4-cyclopentyl-1,3-dimethyl-2-oxo-1,2,3,4-tetrahydroquinoxalin-6-yl]amino}-N-cyclopropylbenzamide;
4-{[(3R)-4-cyclopentyl-1,3-dimethyl-2-oxo-1,2,3,4-tetrahydroquinoxalin-6-yl]amino}-N,N-dimethylbenzenesulphonamide;
4-{[(3R)-4-cyclopentyl-1,3-dimethyl-2-oxo-1,2,3,4-tetrahydroquinoxalin-6-yl]amino}-N-(1-methylpiperidin-4-yl)benzamide;
4-{[(3R)-4-cyclopentyl-1,3-dimethyl-2-oxo-1,2,3,4-tetrahydroquinoxalin-6-yl]oxy}-N-cyclopropylbenzamide;
(3R)-4-cyclopentyl-1,3-dimethyl-6-{[4-(morpholin-4-ylcarbonyl)phenyl]amino}-3,4-dihydroquinoxalin-2(1H)-one;
4-{[(3R)-4-cyclopentyl-1,3-dimethyl-2-oxo-1,2,3,4-tetrahydroquinoxalin-6-yl]amino}-N-isopropylbenzamide;
4-{[(3R)-4-cyclopentyl-1,3-dimethyl-2-oxo-1,2,3,4-tetrahydroquinoxalin-6-yl]amino}-N,N-dimethylbenzamide;
4-{[(3R)-4-benzyl-1,3-dimethyl-2-oxo-1,2,3,4-tetrahydroquinoxalin-6-yl]amino}-N-(oxetan-3-ylmethyl)benzamide;
4-{[(3R)-4-benzyl-1,3-dimethyl-2-oxo-1,2,3,4-tetrahydroquinoxalin-6-yl]amino}-N-cyclopropylbenzamide;
4-{[(3R)-4-benzyl-1,3-dimethyl-2-oxo-1,2,3,4-tetrahydroquinoxalin-6-yl]amino}-N-(1-methylpiperidin-4-yl)benzamide;
(3R)-4-benzyl-1,3-dimethyl-6-{[4-(morpholin-4-ylcarbonyl)phenyl]amino}-3,4-dihydroquinoxalin-2(1H)-one;
(3R)-4-benzyl-1,3-dimethyl-6-{[4-(morpholin-4-ylsulphonyl)phenyl]amino}-3,4-dihydroquinoxalin-2(1H)-one;
4-{[(3R)-4-benzyl-1,3-dimethyl-2-oxo-1,2,3,4-tetrahydroquinoxalin-6-yl]amino}-N,N-dimethylbenzenesulphonamide;
(3R)-4-benzyl-1,3-dimethyl-6-({4-[(4-methylpiperazin-1-yl)sulphonyl]phenyl}amino)-3,4-dihydroquinoxalin-2(1H)-one;
(3R)-4-benzyl-6-({4-[(1,1-dioxido-1-thia-6-azaspiro[3.3]hept-6-yl)carbonyl]phenyl}amino)-1,3-dimethyl-3,4-dihydroquinoxalin-2(1H)-one;
4-{[(3R)-4-(4-methoxybenzyl)-1,3-dimethyl-2-oxo-1,2,3,4-tetrahydroquinoxalin-6-yl]amino}-N-(1-methylpiperidin-4-yl)benzamide;
(3R)-4-(4-methoxybenzyl)-1,3-dimethyl-6-{[4-(morpholin-4-ylcarbonyl)phenyl]amino}-3,4-dihydroquinoxalin-2(1H)-one;
4-{[(3R)-4-(4-methoxybenzyl)-1,3-dimethyl-2-oxo-1,2,3,4-tetrahydroquinoxalin-6-yl]amino}-N-(oxetan-3-ylmethyl)benzamide;
(3R)-4-(4-methoxybenzyl)-1,3-dimethyl-6-{[4-(2-oxa-6-azaspiro[3.3]hept-6-ylcarbonyl)phenyl]amino}-3,4-dihydroquinoxalin-2(1H)-one;
4-{[(3R)-4-(4-methoxybenzyl)-1,3-dimethyl-2-oxo-1,2,3,4-tetrahydroquinoxalin-6-yl]amino}-N-[2-(4-methylpiperazin-1-yl)ethyl]benzamide;
(3R)-4-(4-methoxybenzyl)-1,3-dimethyl-6-[(4-{[4-(propan-2-yl)piperazin-1-yl]carbonyl}phenyl)amino]-3,4-dihydroquinoxalin-2(1H)-one;
4-{[(3R)-4-cycloheptyl-1,3-dimethyl-2-oxo-1,2,3,4-tetrahydroquinoxalin-6-yl]amino}-N-(1-methylpiperidin-4-yl)benzamide;
(3R)-4-cycloheptyl-1,3-dimethyl-6-{[4-(morpholin-4-ylcarbonyl)phenyl]amino}-3,4-dihydroquinoxalin-2(1H)-one;
4-{[(3R)-4-cycloheptyl-1,3-dimethyl-2-oxo-1,2,3,4-tetrahydroquinoxalin-6-yl]amino}-N-(oxetan-3-ylmethyl)benzamide;
4-{[(3R)-1,3-dimethyl-2-oxo-4-(tetrahydro-2H-pyran-4-yl)-1,2,3,4-tetrahydroquinoxalin-6-yl]amino}-N-(1-methylpiperidin-4-yl)benzamide;
(3R)-1,3-dimethyl-6-{[4-(morpholin-4-ylcarbonyl)phenyl]amino}-4-(tetrahydro-2H-pyran-4-yl)-3,4-dihydroquinoxalin-2(1H)-one;
4-{[(3R)-1,3-dimethyl-2-oxo-4-(tetrahydro-2H-pyran-4-yl)-1,2,3,4-tetrahydroquinoxalin-6-yl]amino}-N-(oxetan-3-ylmethyl)benzamide;
(3R)-1,3-dimethyl-6-{[4-(2-oxa-6-azaspiro[3.3]hept-6-ylcarbonyl)phenyl]amino}-4-(tetrahydro-2H-pyran-4-yl)-3,4-dihydroquinoxalin-2(1H)-one;
4-{[(3R)-1,3-dimethyl-2-oxo-4-(tetrahydro-2H-pyran-4-yl)-1,2,3,4-tetrahydroquinoxalin-6-yl]amino}-N-[2-(4-methylpiperazin-1-yl)ethyl]benzamide;
(3R)-6-({4-[(1,1-dioxido-1-thia-6-azaspiro[3.3]hept-6-yl)carbonyl]phenyl}amino)-1,3-dimethyl-4-(tetrahydro-2H-pyran-4-yl)-3,4-dihydroquinoxalin-2(1H)-one;
N-(1-acetylpiperidin-4-yl)-4-{[(3R)-1,3-dimethyl-2-oxo-4-(tetrahydro-2H-pyran-4-yl)-1,2,3,4-tetrahydroquinoxalin-6-yl]amino}benzamide;
(3R)-1,3-dimethyl-6-[(4-{[4-(propan-2-yl)piperazin-1-yl]carbonyl}phenyl)amino]-4-(tetrahydro-2H-pyran-4-yl)-3,4-dihydroquinoxalin-2(1H)-one;
4-{[(3R)-1,3-dimethyl-2-oxo-4-(tetrahydro-2H-pyran-4-yl)-1,2,3,4-tetrahydroquinoxalin-6-yl]amino}-N-(1-methylazetidin-3-yl)benzamide;
N-cyclopropyl-4-{[(3R)-1,3-dimethyl-2-oxo-4-(tetrahydro-2H-pyran-4-yl)-1,2,3,4-tetrahydroquinoxalin-6-yl]amino}benzamide;
4-{[(3R)-1,3-dimethyl-2-oxo-4-(tetrahydro-2H-pyran-4-yl)-1,2,3,4-tetrahydroquinoxalin-6-yl]amino}-3-methoxy-N-(1-methylpiperidin-4-yl)benzamide;
N-{4-[4-(cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydroquinoxalin-6-yl]amino}-3-methoxybenzamide;
(3R)-6-({2-methoxy-4-[(4-methylpiperazin-1-yl)carbonyl]phenyl}amino)-1,3-dimethyl-4-(tetrahydro-2H-pyran-4-yl)-3,4-dihydroquinoxalin-2(1H)-one;
4-{[4-(2,6-difluorobenzyl)-1,3-dimethyl-2-oxo-1,2,3,4-tetrahydroquinoxalin-6-yl]amino}-N-(1-methylpiperidin-4-yl)benzamide;
N-{4-[4-(cyclopropylmethyl)piperazin-1-yl]cyclohexyl}-4-{[4-(2,6-difluorobenzyl)-1,3-dimethyl-2-oxo-1,2,3,4-tetrahydroquinoxalin-6-yl]amino}benzamide;
4-{[(3R)-4-(4-methoxybenzyl)-1,3-dimethyl-2-oxo-1,2,3,4-tetrahydroquinoxalin-6-yl]amino}-N,N-dimethylbenzenesulphonamide;
(3R)-4-(4-methoxybenzyl)-1,3-dimethyl-6-{[4-(morpholin-4-ylsulphonyl)phenyl]amino}-3,4-dihydroquinoxalin-2(1H)-one;
(3R)-4-(4-methoxybenzyl)-1,3-dimethyl-6-({4-[(4-methylpiperazin-1-yl)sulphonyl]phenyl}amino)-3,4-dihydroquinoxalin-2(1H)-one;
(3R)-4-(4-methoxybenzyl)-1,3-dimethyl-6-[(4-{[4-(propan-2-yl)piperazin-1-yl]sulphonyl}phenyl)amino]-3,4-dihydroquinoxalin-2(1H)-one;
4-{[(3R)-4-cycloheptyl-1,3-dimethyl-2-oxo-1,2,3,4-tetrahydroquinoxalin-6-yl]amino}-N,N-dimethylbenzenesulphonamide;
4-{[(3R)-1,3-dimethyl-2-oxo-4-(tetrahydro-2H-pyran-4-yl)-1,2,3,4-tetrahydroquinoxalin-6-yl]amino}-N,N-dimethylbenzenesulphonamide;
(3R)-1,3-dimethyl-6-{[4-(morpholin-4-ylsulphonyl)phenyl]amino}-4-(tetrahydro-2H-pyran-4-yl)-3,4-dihydroquinoxalin-2(1H)-one;
(3R)-1,3-dimethyl-6-({4-[(4-methylpiperazin-1-yl)sulphonyl]phenyl}amino)-4-(tetrahydro-2H-pyran-4-yl)-3,4-dihydroquinoxalin-2(1H)-one,

and the diastereomers, racemates, polymorphs and physiologically acceptable salts thereof.

### DEFINITIONS

C₁-C₆-Alkyl, or a C₁-C₆-alkyl group, is understood to mean a linear or branched, saturated monovalent hydrocarbyl radical, for example a methyl, ethyl, propyl, butyl, pentyl, hexyl, iso-propyl, iso-butyl, sec-butyl, tert-butyl, iso-pentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl or 1,2-dimethylbutyl radical.

Preferably, C₁-C₆-alkyl or a C₁-C₆-alkyl group is understood to mean C₁-C₄-alkyl or C₂-C₅-alkyl, more preferably C₁-C₃-alkyl, i.e. a methyl, ethyl, propyl or isopropyl radical.

C₂-C₅-Alkylene, or a C₂-C₅-alkylene group, is understood to mean a linear or branched, saturated, bivalent hydrocarbyl radical, for example an ethylene, propylene, butylene, pentylene, iso-propylene, iso-butylene, sec-butylene, tert-butylene, iso-pentylene, 2-methylbutylene, 1-methylbutylene, 1-ethylpropylene, 1,2-dimethylpropylene, neo-pentylene or 1,1-dimethylpropylene radical.

C₂-C₆-Alkenyl, or a C₂-C₆-alkenyl group, is understood to mean a linear or branched, monovalent hydrocarbyl radical having one or two C═C double bonds, for example an ethenyl, (E)-prop-2-enyl, (Z)-prop-2-enyl, allyl (prop-1-enyl), allenyl, buten-1-yl or buta-1,3-dienyl radical. Preference is given to C₃-C₆-alkenyl or C₂-C₄-alkenyl, particular preference to ethenyl and allyl.

C₂-C₆-Alkynyl, or a C₂-C₆-alkynyl group, is understood to mean a linear or branched, monovalent hydrocarbyl radical having one CC triple bond, for example an ethynyl, propargyl (prop-1-ynyl) or butyn-1-yl radical. Preference is given to C₃-C₆-alkynyl or C₂-C₄-alkynyl, particular preference to ethynyl and propargyl.

C₁-C₄-Alkoxy, or a C₁-C₄-alkoxy group, is understood to mean a linear or branched, saturated alkyl ether radical —O-alkyl, for example a methoxy, ethoxy, n-propoxy, isopropoxy or tert-butoxy radical.

Preferably, C₁-C₄-alkoxy, or a C₁-C₄-alkoxy group, is understood to mean C₁-C₃-alkoxy, more preferably a methoxy or ethoxy radical.

C₁-C₄-Alkylthio, or a C₁-C₄-alkylthio group, is understood to mean a linear or branched, saturated alkyl thioether radical —S-alkyl, for example a methylthio, ethylthio, n-propylthio, isopropylthio or tert-butylthio radical.

Preferably, C₁-C₄-alkylthio, or a C₁-C₄-alkylthio group, is understood to mean C₁-C₃-alkylthio, more preferably a methylthio or ethylthio radical.

A heteroatom is understood to mean —O—, NH—, ═N— or —S—, including the oxidized forms thereof —S(═O)— and —S(═O)₂— and a sulphoximine —S(═O)(═NH)— derived from —S(═O)₂—. The heteroatom —NH— may optionally be substituted by C₁-C₃-alkyl, C₁-C₃-alkylcarbonyl, C₁-C₄-alkoxycarbonyl, or —S(═O)₂—C₁-C₃-alkyl. The ═NH of the abovementioned sulphoximine may optionally be substituted by C₁-C₃-alkyl, C₁-C₃-alkylcarbonyl, C₁-C₄-alkoxycarbonyl.

Preference is given to an oxygen or nitrogen atom.

Oxo, or an oxo substituent, is understood to mean a double-bonded oxygen atom ═O. Oxo may be bonded to atoms of suitable valency, for example to a saturated carbon atom or to sulphur.

Preference is given to the bond to carbon to form a carbonyl group.

Preference is further given to the bond of two double-bonded oxygen atoms to sulphur to form a sulphonyl group —(S═O)₂—.

Halogen is understood to mean fluorine, chlorine, bromine or iodine.

Fluorine, chlorine, bromine or iodine which is an optional substituent on the phenyl ring may be in the ortho, meta or para position. Preference is given to fluorine or chlorine.

The preferred position is the meta or para position.

A halo-C₁-C₄-alkyl radical is understood to mean a C₁-C₄-alkyl radical having at least one halogen substituent, preferably having at least one fluorine substituent.

Preference is given to fluoro-C₁-C₃-alkyl radicals, for example difluoromethyl, trifluoromethyl, 2,2,2-trifluoroethyl or pentafluoroethyl.

Particular preference is given to perfluorinated alkyl radicals such as trifluoromethyl or pentafluoroethyl.

Phenyl-C₁-C₃-alkyl is understood to mean a group composed of an optionally substituted phenyl radical and a C₁-C₃-alkyl group, and which is bonded to the rest of the molecule via the C₁-C₃-alkyl group.

A halo-C₁-C₄-alkoxy radical is understood to mean a C₁-C₄-alkoxy radical having at least one halogen substituent, preferably having at least one fluorine substituent.

Preference is given to fluoro-C₁-C₃-alkoxy radicals, for example difluoromethoxy, trifluoromethoxy or 2,2,2-trifluoroethoxy radicals.

A halo-C₁-C₄-alkylthio radical is understood to mean a C₁-C₄-alkylthio radical having at least one halogen substituent, preferably having at least one fluorine substituent.

Preference is given to fluoro-C₁-C₃-alkylthio radicals, especially trifluoromethylthio.

A C₁-C₃-alkylcarbonyl radical is understood to mean a C₁-C₃-alkyl-C(═O) group. Preference is given to acetyl or propanoyl.

A C₁-C₄-alkoxycarbonyl radical is understood to mean a C₁-C₄-alkoxy-C(═O) group. Preference is given to methoxycarbonyl, ethoxycarbonyl or tert-butoxycarbonyl.

Aryl is understood to mean an unsaturated, fully conjugated system which is formed from carbon atoms and has 3, 5 or 7 conjugated double bonds, for example phenyl, naphthyl or phenanthryl. Preference is given to phenyl.

Heteroaryl is understood to mean ring systems which have an aromatically conjugated ring system and contain at least one and up to five heteroatoms as defined above. These ring systems may have 5, 6 or 7 ring atoms, or else, in the case of fused or benzofused ring systems, combinations of 5- and 6-membered ring systems, 5- and 5-membered ring systems, or else 6- and 6-membered ring systems. Examples include ring systems such as pyrrolyl, pyrazolyl, imidazolyl, triazolyl, tetrazolyl, furyl, thienyl, oxazolyl, thiazolyl, isoxazolyl, oxadiazolyl, thiadiazolyl, pyridinyl, pyrimidinyl, pyrazinyl, triazinyl, oxazinyl, indolyl, benzimidazolyl, indazolyl, benzotriazolyl, benzothiazolyl, benzoxazolyl, benzofuryl, benzothienyl, quinolinyl, isoquinolinyl, cinnolinyl, quinazolinyl, quinoxalinyl, imidazopyridinyl or else benzoxazinyl.

Preference is given to 5- to 6-membered, monocyclic heteroaryl, for example pyrrolyl, pyrazolyl, imidazolyl, triazolyl, tetrazolyl, furyl, thienyl, oxazolyl, thiazolyl, isoxazolyl, oxadiazolyl, thiadiazolyl, pyridinyl, pyrimidinyl, pyrazinyl, triazinyl.

C₃-C₆-Cycloalkyl, C₃-C₇-cycloalkyl, C₃-C₈-cycloalkyl, C₅-C₇-cycloalkyl, and C₅-C₈-cycloalkyl are understood to mean a monocyclic, saturated ring system formed exclusively from carbon atoms and having, respectively, 3 to 6, 3 to 7, 3 to 8, 5 to 7, and 5 to 8 atoms. Examples are cyclopropyl, cyclobutyl, cyclopentyl, cyclohexyl, cycloheptyl or cyclooctyl.

C₄-C₆-Cycloalkenyl, C₄-C₈-cycloalkenyl, and C₅-C₈-cycloalkenyl are understood to mean a monocyclic, mono- or polyunsaturated, nonaromatic ring system formed exclusively from carbon atoms and having, respectively, 4 to 6, 4 to 8, and 5 to 8 atoms. Examples are cyclobuten-1-yl, cyclopenten-1-yl, cyclohexen-2-yl, cyclohexen-1-yl or cycloocta-2,5-dienyl.

C₃-C₆-Cycloalkyl-C₁-C₃-alkyl or a C₃-C₆-cycloalkyl-C₁-C₃-alkyl group is understood to mean a group which is composed of C₃-C₆-cycloalkyl as defined above and a C₁-C₃-alkyl group, and which is bonded to the rest of the molecule via the C₁-C₃-alkyl group. Preference is given to C₃-C₆-cycloalkylmethyl, particular preference to cyclopropylmethyl.

Heterocycloalkyl is understood to mean a 4- to 8-membered monocyclic, saturated ring system having 1 to 3 heteroatoms as defined above in any combination. Preference is given to 4- to 7-membered heterocycloalkyl groups, particular preference to 5- to 6-membered heterocycloalkyl groups. Examples include pyrrolidinyl, piperidinyl, tetrahydrofuranyl, tetrahydropyranyl, oxetanyl, azetidinyl, azepanyl, morpholinyl, thiomorpholinyl or piperazinyl.

Heterocycloalkenyl is understood to mean a 4- to 8-membered monocyclic, mono- or polyunsaturated, nonaromatic ring system having 1 to 3 heteroatoms as defined above in any combination. Preference is given to 4-7-membered heterocycloalkyl groups, particular preference to 5-6-membered heterocycloalkyl groups. Examples include 4H-pyranyl, 2H-pyranyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl, or 4H-[1,4]thiazinyl.

C₅-C₁₁-Spirocycloalkyl or C₅-C₁₁-heterospirocycloalkyl having a replacement of 1-4 carbon atoms by heteroatoms as defined above in any combination is understood to mean a fusion of two saturated ring systems which share a common atom. Examples are spiro[2.2]pentyl, spiro[2.3]hexyl, azaspiro[2.3]hexyl, spiro[3.3]heptyl, azaspiro[3.3]heptyl, oxaazaspiro[3.3]heptyl, thiaazaspiro[3.3]heptyl, oxaspiro[3.3]heptyl, oxazaspiro[5.3]nonyl, oxazaspiro[4.3]octyl, oxazaspiro[5.5]undecyl, diazaspiro[3.3]heptyl, thiazaspiro[3.3]heptyl, thiazaspiro[4.3]octyl, azaspiro[5.5]decyl, and the further homologous spiro[3.4], spiro[4.4], spiro[5.5], spiro[6.6], spiro[2.4], spiro[2.5], spiro[2.6], spiro[3.5], spiro[3.6], spiro[4.5], spiro[4.6] and spiro[5.6] systems including the variants modified by heteroatoms as per the definition. Preference is given to C₆-C₈-heterospirocycloalkyl.

C₆-C₁₂-Bicycloalkyl or C₆-C₁₂-heterobicycloalkyl having a replacement of 1-4 carbon atoms by heteroatoms as defined above in any combination is understood to mean a fusion of two saturated ring systems which share two directly adjacent atoms. Examples are bicyclo[2.2.0]hexyl, bicyclo[3.3.0]octyl, bicyclo[4.4.0]decyl, bicyclo[5.4.0]undecyl, bicyclo[3.2.0]heptyl, bicyclo[4.2.0]octyl, bicyclo[5.2.0]nonyl, bicyclo[6.2.0]decyl, bicyclo[4.3.0]nonyl, bicyclo[5.3.0]decyl, bicyclo[6.3.0]undecyl and bicyclo[5.4.0]undecyl, including the variants modified by heteroatoms, for example azabicyclo[3.3.0]octyl, azabicyclo[4.3.0]nonyl, diazabicyclo[4.3.0]nonyl, oxazabicyclo[4.3.0]nonyl, thiazabicyclo[4.3.0]nonyl or azabicyclo[4.4.0]decyl, and the further possible combinations as per the definition. Preference is given to C₆-C₁₀-heterobicycloalkyl.

A bridged C₆-C₁₂ ring system such as bridged C₆-C₁₂-cycloalkyl or bridged C₆-C₁₂-heterocycloalkyl is understood to mean a fusion of at least two saturated rings which share two atoms that are not directly adjacent. This may give rise either to a bridged carbocycle (bridged cycloalkyl) or to a bridged heterocycle (bridged heterocycloalkyl) having a replacement of 1-4 carbon atoms by heteroatoms as defined above in any combination. Examples are bicyclo[2.2.1]heptyl, azabicyclo[2.2.1]heptyl, oxazabicyclo[2.2.1]heptyl, thiazabicyclo[2.2.1]heptyl, diazabicyclo[2.2.1]heptyl, bicyclo[2.2.2]octyl, azabicyclo[2.2.2]octyl, diazabicyclo[2.2.2]octyl, oxazabicyclo[2.2.2]octyl, thiazabicyclo[2.2.2]octyl, bicyclo[3.2.1]octyl, azabicyclo[3.2.1]octyl, diazabicyclo[3.2.1]octyl, oxazabicyclo[3.2.1]octyl, thiazabicyclo[3.2.1]octyl, bicyclo[3.3.1]nonyl, azabicyclo[3.3.1]nonyl, diazabicyclo[3.3.1]nonyl, oxazabicyclo[3.3.1]nonyl, thiazabicyclo[3.3.1]nonyl, bicyclo[4.2.1]nonyl, azabicyclo[4.2.1]nonyl, diazabicyclo[4.2.1]nonyl, oxazabicyclo[4.2.1]nonyl, thiazabicyclo[4.2.1]nonyl, bicyclo[3.3.2]decyl, azabicyclo[3.3.2]decyl, diazabicyclo[3.3.2]decyl, oxazabicyclo[3.3.2]decyl, thiazabicyclo[3.3.2]decyl or azabicyclo[4.2.2]decyl and the further possible combinations according to the definition.

Preference is given to bridged C₆-C₁₀-heterocycloalkyl.

Inventive compounds are the compounds of the general formula (I) and the salts, solvates and solvates of the salts thereof, the compounds encompassed by the general formula (I) of the formulae specified hereinafter and the salts, solvates and solvates of the salts thereof, and the compounds encompassed by the general formula (I) and specified hereinafter as working examples and the salts, solvates and solvates of the salts thereof, to the extent that the compounds encompassed by the general formula (I) and specified hereinafter are not already salts, solvates and solvates of the salts.

The present invention is likewise considered to encompass the use of the salts of the inventive compounds.

In the context of the present invention, preferred salts are physiologically acceptable salts of the inventive compounds. Also included, however, are salts which are themselves unsuitable for pharmaceutical applications but can be used, for example, for the isolation or purification of the inventive compounds.

Physiologically acceptable salts of the inventive compounds include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

The present invention further provides all the possible crystalline and polymorphous forms of the inventive compounds, where the polymorphs may be present either as single polymorphs or as a mixture of a plurality of polymorphs in all concentration ranges.

The present invention also relates to medicaments comprising the inventive compounds together with at least one or more than one further active ingredients, especially for prophylaxis and/or treatment of neoplastic disorders.

In the context of the invention, solvates refer to those forms of the inventive compounds which, in the solid or liquid state, form a complex by coordination with solvent molecules. Hydrates are a specific form of the solvates in which the coordination is with water. Preferred solvates in the context of the present invention are hydrates.

Where the inventive compounds can occur in tautomeric forms, the present invention encompasses all the tautomeric forms.

The present invention also encompasses all suitable isotopic variants of the inventive compounds. An isotopic variant of an inventive compound is understood here to mean a compound in which at least one atom within the inventive compound has been exchanged for another atom of the same atomic number, but with a different atomic mass from the atomic mass which usually or predominantly occurs in nature. Examples of isotopes which can be incorporated into an inventive compound are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of an inventive compound, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the inventive compounds may therefore in some cases also constitute a preferred embodiment of the present invention. Isotopic variants of the inventive compounds can be prepared by the processes known to those skilled in the art, for example by the methods described below and the instructions reproduced in the working examples, by using corresponding isotopic modifications of the particular reagents and/or starting compounds.

Depending on their structure, the inventive compounds may exist in different stereoisomeric forms, i.e. in the form of configurational isomers or if appropriate also as conformational isomers. The inventive compounds may have a centre of asymmetry at the carbon atom to which R⁵ and R⁶ are bonded (C-3). They may therefore take the form of pure enantiomers, racemates, or else of diastereomers or mixtures thereof when one or more of the substituents described in the formula (I) contains a further element of asymmetry, for example a chiral carbon atom. The present invention therefore also encompasses diastereomers and the respective mixtures thereof. The pure stereoisomers can be isolated from such mixtures in a known manner; chromatography processes are preferably used for this, in particular HPLC chromatography on a chiral or achiral phase.

In general, the inventive enantiomers inhibit the target proteins to different degrees and have different activity in the cancer cell lines studied. The more active enantiomer is preferred, which is often that in which the centre of asymmetry represented by the carbon atom bonded to R⁵ and R⁶ has (R) configuration.

The present invention further provides enantiomer mixtures of the (3R)-configured inventive compounds with their (3S) enantiomers, especially the corresponding racemates and enantiomer mixtures in which the (3R) form predominates.

The inventive compounds can act systemically and/or locally. For this purpose, it can be administered in a suitable manner, for example by the oral, parenteral, pulmonary, nasal, sublingual, lingual, buccal, rectal, dermal, transdermal, conjunctival, otic route, or as an implant or stent.

The inventive compounds can be administered in administration forms suitable for these administration routes.

Suitable administration forms for oral administration are those which release the inventive compounds in a rapid and/or modified manner, work according to the prior art and contain the inventive compounds in crystalline and/or amorphous and/or dissolved form, for example tablets (uncoated or coated tablets, for example with enteric or retarded-dissolution or insoluble coatings which control the release of the inventive compound), tablets or films/wafers which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can bypass an absorption step (e.g. intravenously, intraarterially, intracardially, intraspinally or intralumbally) or include an absorption (e.g. intramuscularly, subcutaneously, intracutaneously, percutaneously or intraperitoneally). Suitable administration forms for parenteral administration include injection and infusion formulations in the form of solutions, suspensions, emulsions, lyophilizates or sterile powders.

For the other administration routes, suitable examples are inhalation medicaments (including powder inhalers, nebulizers), nasal drops, solutions or sprays; tablets for lingual, sublingual or buccal administration, films/wafers or capsules, suppositories, ear or eye preparations, vaginal capsules, aqueous suspensions (lotions, shaking mixtures), lipophilic suspensions, ointments, creams, transdermal therapeutic systems (for example patches), milk, pastes, foams, dusting powders, implants or stents.

The inventive compounds can be converted to the administration forms mentioned. This can be done in a manner known per se, by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), dyes (e.g. inorganic pigments, for example iron oxides) and taste and/or odour correctants.

The present invention further provides medicaments which comprise the inventive compounds, typically together with one or more inert, nontoxic, pharmaceutically suitable excipients, and the use thereof for the aforementioned purposes.

The formulation of the inventive compounds to give pharmaceutical preparations is effected in a manner known per se, by converting the active ingredient(s) to the desired administration form with the excipients customary in pharmaceutical formulation.

The excipients used may, for example, be carrier substances, fillers, disintegrants, binders, humectants, glidants, absorbents and adsorbents, diluents, solvents, cosolvents, emulsifiers, solubilizers, taste correctants, colorants, preservatives, stabilizers, wetting agents, salts for modifying osmotic pressure or buffers. Reference should be made to Remington's Pharmaceutical Science, 15th ed. Mack Publishing Company, East Pennsylvania (1980).

The pharmaceutical formulations may be in solid form, for example in the form of tablets, coated tablets, pills, suppositories, capsules, transdermal systems, or in semisolid form, for example as ointments, creams, gels, suppositories, emulsions, or in liquid form, for example as solutions, tinctures, suspensions or emulsions.

The excipients used in the context of the invention may, for example, be salts, saccharides (mono-, di-, tri-, oligo- and/or polysaccharides), proteins, amino acids, peptides, fats, waxes, oils, hydrocarbons and derivatives thereof, and the excipients may be of natural origin or synthetic or partially synthetic.

Useful forms for oral or peroral administration are especially tablets, coated tablets, capsules, pills, powders, granules, pastilles, suspensions, emulsions or solutions.

Useful forms for parenteral administration are especially suspensions, emulsions, and particularly solutions.

The inventive compounds are suitable for prophylaxis and/or treatment of hyperproliferative disorders, for example psoriasis, keloids and other hyperplasias which affect the skin, benign prostate hyperplasias (BPH), solid tumours and haematological tumours.

Solid tumours that can be treated in accordance with the invention are, for example, tumours of the breast, the respiratory tract, the brain, the reproductive organs, the gastrointestinal tract, the urogenital tract, the eye, the liver, the skin, the head and the neck, the thyroid gland, the parathyroid gland, the bones, and the connective tissue and metastases of these tumours.

Haematological tumours that can be treated are, for example, multiple myeloma, lymphoma or leukaemia.

Breast tumours that can be treated are, for example, mammary carcinoma with positive hormone receptor status, mammary carcinoma with negative hormone receptor status, Her-2-positive mammary carcinoma, hormone receptor- and Her-2-negative mammary carcinoma, BRCA-associated mammary carcinoma and inflammatory mammary carcinoma.

Tumours of the respiratory tract that can be treated are, for example, non-small-cell bronchial carcinoma and small-cell bronchial carcinoma.

Brain tumours that can be treated are, for example, glioma, glioblastoma, astrocytoma, meningioma and medulloblastoma.

Tumours of the male reproductive organs that can be treated are, for example, prostate carcinoma, malignant epididymal tumours, malignant testicular tumours and penile carcinoma.

Tumours of the female reproductive organs that can be treated are, for example, endometrial carcinoma, cervical carcinoma, ovarian carcinoma, vaginal carcinoma and vulvar carcinoma.

Tumours of the gastrointestinal tract that can be treated are, for example, colorectal carcinoma, anal carcinoma, gastric carcinoma, pancreatic carcinoma, oesophagal carcinoma, gallbladder carcinoma, small-intestinal carcinoma, salivary gland carcinoma, neuroendocrine tumours and gastrointestinal stromal tumours.

Tumours of the urogenital tract that can be treated are, for example, urinary bladder carcinoma, renal cell carcinoma, and carcinoma of the renal pelvis and of the urinary tract.

Tumours of the eye that can be treated are, for example, retinoblastoma and intraocular melanoma.

Tumours of the liver that can be treated are, for example, hepatocellular carcinoma and cholangiocellular carcinoma.

Tumours of the skin that can be treated are, for example, malignant melanoma, basalioma, spinalioma, Kaposi's sarcoma and Merkel cell carcinoma.

Tumours of the head and neck that can be treated are, for example, laryngeal carcinoma and carcinoma of the pharynx and of the oral cavity.

Sarcomas that can be treated are, for example, soft tissue sarcoma and osteosarcoma.

Lymphomas that can be treated are, for example, non-Hodgkin's lymphoma, Hodgkin's lymphoma, cutaneous lymphoma, lymphoma of the central nervous system and AIDS-associated lymphoma.

Leukaemias that can be treated are, for example, acute myeloid leukaemia, chronic myeloid leukaemia, acute lymphatic leukaemia, chronic lymphatic leukaemia and hair cell leukaemia.

Advantageously, the inventive compounds can be used for prophylaxis and/or treatment of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

Particularly advantageously, the inventive compounds can be used for prophylaxis and/or treatment of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The inventive compounds are also suitable for prophylaxis and/or treatment of benign hyperproliferative diseases, for example endometriosis, leiomyoma and benign prostate hyperplasia.

The inventive compounds are also suitable for prophylaxis and/or treatment of systemic inflammatory diseases, especially LPS-induced endotoxic shock and/or bacteria-induced sepsis.

The inventive compounds are also suitable for prophylaxis and/or treatment of inflammatory or autoimmune disorders, for example:
pulmonary disorders associated with inflammatory, allergic and/or proliferative processes: chronic obstructive pulmonary disorders of any origin, particularly bronchial asthma; bronchitis of different origin; all forms of restrictive pulmonary disorders, particularly allergic alveolitis; all forms of pulmonary oedema, particularly toxic pulmonary oedema; sarcoidoses and granulomatoses, particularly Boeck's disease
rheumatic disorders/autoimmune disorders/joint disorders associated with inflammatory, allergic and/or proliferative processes: all forms of rheumatic disorders, especially rheumatoid arthritis, acute rheumatic fever, polymyalgia rheumatica; reactive arthritis; inflammatory soft-tissue disorders of other origin; arthritic symptoms in the case of degenerative joint disorders (arthroses); traumatic arthritis; collagenoses of any origin, e.g. systemic lupus erythematosus, sclerodermia, polymyositis, dermatomyositis, Sjögren's syndrome, Still's syndrome, Felty's syndrome
allergies associated with inflammatory and/or proliferative processes: all forms of allergic reactions, e.g. angiooedema, hay fever, insect bites, allergic reactions to medicaments, blood derivatives, contrast agents, etc., anaphylactic shock, urticaria, contact dermatitis
vascular inflammation (vasculitis): panarteritis nodosa, temporal arteritis, erythema nodosum
dermatological disorders associated with inflammatory, allergic and/or proliferative processes: atopic dermatitis; psoriasis; pityriasis rubra pilaris; erythematous disorders triggered by different noxae, for example radiation, chemicals, burns, etc.; bullous dermatoses; lichenoid disorders; pruritus; seborrhoeic eczema; rosacea; pemphigus vulgaris; erythema exsudativum multiforme; balanitis; vulvitis; hair loss, such as alopecia areata; cutaneous T-cell lymphoma
renal disorders associated with inflammatory, allergic and/or proliferative processes: nephrotic syndrome; all nephritides
hepatic disorders associated with inflammatory, allergic and/or proliferative processes: acute hepatic disintegration; acute hepatitis of different origin, for example viral, toxic, medicament-induced; chronic aggressive and/or chronic intermittent hepatitis
gastrointestinal disorders associated with inflammatory, allergic and/or proliferative processes: regional enteritis (Crohn's disease); ulcerative colitis; gastritis; reflux oesophagitis; gastroenteritides of other origin, z.B. indigenous sprue
proctological disorders associated with inflammatory, allergic and/or proliferative processes: anal eczema; fissures; haemorrhoids; idiopathic proctitis
ocular disorders associated with inflammatory, allergic and/or proliferative processes: allergic keratitis, uveitis, iritis; conjunctivitis; blepharitis; optic neuritis; chlorioditis; sympathetic ophthalmia
disorders of the ear-nose-throat region associated with inflammatory, allergic and/or proliferative processes: allergic rhinitis, hay fever; otitis externa, for example caused by contact eczema, infection, etc.; otitis media
neurological disorders associated with inflammatory, allergic and/or proliferative processes: cerebral oedema, particularly tumour-related cerebral oedema; multiple sclerosis; acute encephalomyelitis; meningitis; various forms of seizure, for example West's syndrome
haematological disorders associated with inflammatory, allergic and/or proliferative processes: congenital haemolytic anaemia; idiopathic thrombocytopenia
neoplastic disorders associated with inflammatory, allergic and/or proliferative processes: acute lymphatic leukaemia; malignant lymphoma; lymphogranulomatoses; lymphosarcoma; extensive metastases, particularly in the case of mammary, bronchial and prostate carcinoma
endocrine disorders associated with inflammatory, allergic and/or proliferative processes: endocrine orbitopathy; thyrotoxic crisis; de Quervain's thyroiditis; Hashimoto's thyroiditis; Basedow's disease
organ and tissue transplants, graft-versus-host disease
severe states of shock, for example anaphylactic shock, systemic inflammatory response syndrome (SIRS)
substitution therapy in the case of: congenital primary renal insufficiency, for example congenital adrenogenital syndrome; acquired primary renal insufficiency, for example Addison's disease, autoimmune adrenalitis, postinfectious tumours, metastases, etc; congenital secondary renal insufficiency, for example congenital hypopituitarism; acquired secondary renal insufficiency, for example postinfectious tumours, etc.
emesis associated with inflammatory, allergic and/or proliferative processes, for example in combination with a 5-HT3 antagonist in the case of cytostatic-induced nausea
pain of inflammatory origin, for example lumbago

The inventive compounds are also suitable for the treatment of viral disorders, for example infections caused by papillomaviruses, herpesviruses, Epstein-Barr viruses, hepatitis B or C viruses, and human immunodeficiency viruses.

The inventive compounds are also suitable for the treatment of atherosclerosis, dyslipidaemia, hypercholesterolaemia, hypertriglyceridaemia, peripheral vascular disorders, cardiovascular disorders, angina pectoris, ischaemia, stroke, myocardial infarction, angioplastic restenosis, hypertension, thrombosis, obesity, endotoxaemia.

The inventive compounds are also suitable for the treatment of neurodegenerative diseases, for example multiple sclerosis, Alzheimer's disease and Parkinson's disease.

These disorders are well-characterized in man, but also exist in other mammals.

The present application further provides the inventive compounds for use as medicaments, especially for prophylaxis and/or treatment of neoplastic disorders.

The present application further provides the inventive compounds for prophylaxis and/or treatment of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present application further provides the inventive compounds for prophylaxis and/or treatment of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The invention further provides for the use of the inventive compounds for production of a medicament.

The present application further provides for the use of the inventive compounds for production of a medicament for prophylaxis and/or treatment of neoplastic disorders.

The present application further provides for the use of the inventive compounds for production of a medicament for prophylaxis and/or treatment of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present application further provides for the use of the inventive compounds for production of a medicament for prophylaxis and/or treatment of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The present application further provides for the use of the inventive compounds for prophylaxis and/or treatment of neoplastic disorders.

The present application further provides for the use of the inventive compounds for prophylaxis and/or treatment of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present application further provides for the use of the inventive compounds for prophylaxis and/or treatment of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The present application further provides pharmaceutical formulations in the form of tablets comprising one of the inventive compounds for prophylaxis and/or treatment of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present application further provides pharmaceutical formulations in the form of tablets comprising one of the inventive compounds for prophylaxis and/or treatment of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The invention further provides for the use of the inventive compounds for treatment of disorders associated with proliferative processes.

The invention further provides for the use of the inventive compounds for treatment of benign hyperplasias, inflammation disorders, autoimmune disorders, sepsis, viral infections, vascular disorders and neurodegenerative disorders.

The inventive compounds can be used alone or, if required, in combination with one or more further pharmacologically active substances, provided that this combination does not lead to undesirable and unacceptable side effects. The present invention therefore further provides medicaments comprising an inventive compound and one or more further active ingredients, especially for prophylaxis and/or treatment of the aforementioned disorders.

For example, the inventive compounds can be combined with known antihyperproliferative, cytostatic or cytotoxic chemical and biological substances for treatment of cancer. The combination of the inventive compounds with other substances commonly used for cancer treatment, or else with radiotherapy, is particularly appropriate.

An illustrative but nonexhaustive list of suitable combination active ingredients is as follows:

abiraterone acetate, abraxane, acolbifene, Actimmune, actinomycin D (dactinomycin), afatinib, affinitak, Afinitor, aldesleukin, alendronic acid, alfaferone, alitretinoin, allopurinol, Aloprim, Aloxi, alpharadin, altretamine, aminoglutethimide, aminopterin, amifostine, amrubicin, amsacrine, anastrozole, anzmet, apatinib, Aranesp, arglabin, arsenic trioxide, Aromasin, arzoxifen, asoprisnil, L-asparaginase, atamestane, atrasentane, avastin, axitinib, 5-azacytidine, azathioprine, BCG or Tice BCG, bendamustine, bestatin, beta-methasone acetate, betamethasone sodium phosphate, bexarotene, bicalutamide, bleomycin sulphate, broxuridine, bortezomib, bosutinib, busulfan, cabazitaxel, calcitonin, campath, camptothecin, capecitabine, carboplatin, carfilzomib, carmustine, casodex, CCI-779, CDC-501, cediranib, cefesone, celebrex, celmoleukin, cerubidine, cediranib, chlorambucil, cisplatin, cladribine, clodronic acid, clofarabine, colaspase, corixa, crisnatol, crizotinib, cyclophosphamide, cyproterone acetate, cytarabine, dacarbazine, dactinomycin, dasatinib, daunorubicin, DaunoXome, Decadron, Decadron Phosphate, decitabine, degarelix, Delestrogen, denileukin diftitox, depomedrol, deslorelin, dexrazoxane, diethylstilbestrol, diflucan, 2′,2′-difluorodeoxycytidine, DN-101, docetaxel, doxifluridine, doxorubicin (Adriamycin), dronabinol, dSLIM, dutasteride, DW-166HC, edotecarin, eflornithine, Eligard, Elitek, Ellence, Emend, enzalutamide, epirubicin, epoetin-alfa, Epogen, epothilone and derivatives thereof, eptaplatin, ergamisol, erlotinib, erythro-hydroxynonyladenine, estrace, oestradiol, oestramustine sodium phosphate, ethinyloestradiol, Ethyol, etidronic acid, etopophos, etoposide, everolimus, exatecan, exemestane, fadrozole, fareston, fenretinide, filgrastim, finasteride, fligrastim, floxuridine, fluconazole, fludarabine, 5-fluorodeoxyuridine monophosphate, 5-fluorouracil (5-FU), fluoxymesterone, flutamide, folotiyn, formestane, fosteabine, fotemustine, fulvestrant, Gammagard, gefitinib, gemcitabine, gemtuzumab, Gleevec, Gliadel, goserelin, gossypol, granisetrone hydrochloride, hexamethylmelamine, histamine dihydrochloride, histrelin, holmium-166-DOTPM, hycamtin, hydrocortone, erythro-hydroxynonyladenine, hydroxyurea, hydroxyprogesterone caproate, ibandronic acid, ibritumomab tiuxetan, idarubicin, ifosfamide, imatinib, iniparib, interferon-alpha, interferon-alpha-2, interferon-alpha-2α, interferon-alpha-2β, interferon-alpha-n1, interferon-alpha-n3, interferon-beta, interferon-gamma-1α, interleukin-2, intron A, iressa, irinotecan, ixabepilone, keyhole limpet haemocyanin, kytril, lanreotide, lapatinib, lasofoxifene, lenalidomide, lentinan sulphate, lestaurtinib, letrozole, leucovorin, leuprolide, leuprolide acetate, levamisole, levofolic acid calcium salt, levothroid, levoxyl, Libra, liposomal MTP-PE, lomustine, lonafarnib, lonidamine, marinol, mechlorethamine, mecobalamine, medroxyprogesterone acetate, megestrol acetate, melphalan, Menest, 6-mercaptopurine, mesna, methotrexate, metvix, miltefosine, minocycline, minodronate, miproxifen, mitomycin C, mitotan, mitoxantrone, modrenal, MS-209, MX-6, myocet, nafarelin, nedaplatin, nelarabine, nemorubicin, neovastat, neratinib, neulasta, neumega, neupogen, nilotimib, nilutamide, nimustine, nolatrexed, nolvadex, NSC-631570, obatoclax, oblimersen, OCT-43, octreotide, olaparib, ondansetron hydrochloride, Onco-TCS, Orapred, osidem, oxaliplatin, paclitaxel, pamidronate disodium, pazopanib, pediapred, pegaspargase, pegasys, pemetrexed, pentostatin, N-phosphonoacetyl-L-aspartate, picibanil, pilocarpine hydrochloride, pirarubicin, plerixafor, plicamycin, PN-401, porfimer sodium, prednimustine, prednisolone, prednisone, Premarin, procarbazine, Procrit, QS-21, quazepam, R-1589, raloxifene, raltitrexed, ranpirnas, RDEA119, Rebif, regorafenib, 13-cis-retinoic acid, rhenium-186 etidronate, rituximab, roferon-A, romidepsin, romurtide, ruxolitinib, salagen, salinomycin, sandostatin, sargramostim, satraplatin, semaxatinib, semustine, seocalcitol, sipuleucel-T, sizofiran, sobuzoxan, Solu-Medrol, sorafenib, streptozocin, strontium-89 chloride, sunitinib, Synthroid, T-138067, tamoxifen, tamsulosin, Tarceva, tasonermin, testolactone, Taxoprexin, Taxoter, teceleukin, temozolomide, temsirolimus, teniposide, testosterone propionate, Testred, thalidomide, thymosin alpha-1, thioguanine, thioTEPA, thyrotropin, tiazofurin, tiludronic acid, tipifarnib, tirapazamine, TLK-286, toceranib, topotecan, toremifen, tositumomab, tastuzumab, treosulfan, transMID-107R, tretinoin, Trexall, trimethylmelamine, trimetrexate, triptorelin acetate, triptorelin pamoate, trofosfamide, UFT, uridine, valrubicin, valspodar, vandetanib, vapreotide, vatalanib, vemurafinib, verte-porfin, vesnarinone, vinblastine, vincristine, vindesine, vinflunine, vinorelbine, virulizin, vismodegib, Xeloda, Z-100, Zinecard, zinostatin stimalamer, zofran, zoledronic acid.

More particularly, the inventive compounds can be combined with antibodies, for example aflibercept, alemtuzumab, bevacizumab, brentuximumab, catumaxomab, cetuximab, denosumab, edrecolomab, gemtuzumab, ibritumomab, ipilimumab, ofatumumab, panitumumab, pertuzumab, rituximab, tositumumab or trastuzumab, and also with recombinant proteins.

More particularly, the inventive compounds can be used in combination with treatments directed against angiogenesis, for example bevacizumab, axitinib, regorafenib, cediranib, sorafenib, sunitinib, lenalidomide or thalidomide.

Combinations with antihormones and steroidal metabolic enzyme inhibitors are particularly suitable because of their favourable profile of side effects.

Combinations with P-TEFb inhibitors are likewise particularly suitable because of the possible synergistic effects.

Generally, the following aims can be pursued with the combination of the inventive compounds with other cytostatically or cytotoxically active agents:
improved efficacy in slowing the growth of a tumour, in reducing its size or even in completely eliminating it, compared with treatment with an individual active ingredient;
the possibility of using the chemotherapeutics used in a lower dosage than in the case of monotherapy;
the possibility of a more tolerable therapy with fewer side effects compared with individual administration;
the possibility of treatment of a broader spectrum of neoplastic disorders;
the attainment of a higher rate of response to the therapy;
a longer survival time of the patient compared with present standard therapy.

In addition, the inventive compounds can also be used in conjunction with radiotherapy and/or surgical intervention.

Preparation of the Inventive Compounds:

In the present description:

NMR signals are reported with their respectively apparent multiplicities or combinations thereof. In this context, s=singlet, d=doublet, t=triplet, q=quartet, qi=quintet, sp=septet, m=multiplet, b=broad signal. Signals having combined multiplicities are reported, for example, as dd=doublet of doublets.
CDCl₃ deuterochloroform
dba dibenzylideneacetone
DMF N,N-dimethylformamide
DMSO-d6 deuterated dimethyl sulphoxide
DMSO dimethyl sulphoxide
HATU (7-aza-1H-benzotriazol-1-yl)-1,1,3,3-tetramethyluronium hexafluorophosphate
RP-HPLC reverse-phase high-pressure liquid chromatography
RT room temperature
Rt retention time
ACN acetonitrile
THF tetrahydrofuran
HBTU O-benzotriazole-N,N,N′,N′-tetramethyluronium hexafluorophosphate
PyBOB (benzotriazol-1-yl)oxytripyrrolidinophosphonium hexafluorophosphate
T3P 2,4,6-tripropyl-1,3,5,2,4,6-trioxatriphosphorinane 2,4,6-trioxide
KOtBu potassium tert-butoxide
LiHMDS lithium bis(trimethylsilyl)amide
KHMDS potassium bis(trimethylsilyl)amide
LCMS liquid chromatography coupled with mass spectrometry
EA ethyl acetate
TFA trifluoroacetic acid
CHAPS 3-{dimethyl[3-(4-{5,9,16-trihydroxy-2,15-dimethyltetracyclo-[8.7.0.0^{2,7}.0^{11,15}]heptadecan-14-yl}pentanamido)propyl]-azaniumyl}propane-1-sulphonate
(+)-BINAP (R)-(+)-2,2′-bis(diphenylphosphino)-1,1′-binaphthyl
(±)-BINAP 2,2′-bis(diphenylphosphino)-1,1′-binaphthyl (racemic)
TBTU (benzotriazol-1-yloxy)bisdimethylaminomethylium fluoroborate
DCC dicyclohexylcarbodiimide

General Description of the Preparation of the Inventive Compounds of the General Formula (I):

The inventive compounds of the formulae (Ia) and (Ib) shown in Scheme 1 can be prepared via synthesis routes described hereinafter. The formulae specified represent different portions of the general formula (I) in which A, R², R³, R⁴, R⁵, R⁶, R⁷, R⁸, R⁹ and n are each as defined for the general formula (I). In compounds of the formula (Ia), a —C(═O)NR⁸R⁹ group replaces R¹; in compounds of the formula (Ib), a —S(═O)₂NR⁸R⁹ group replaces R¹.

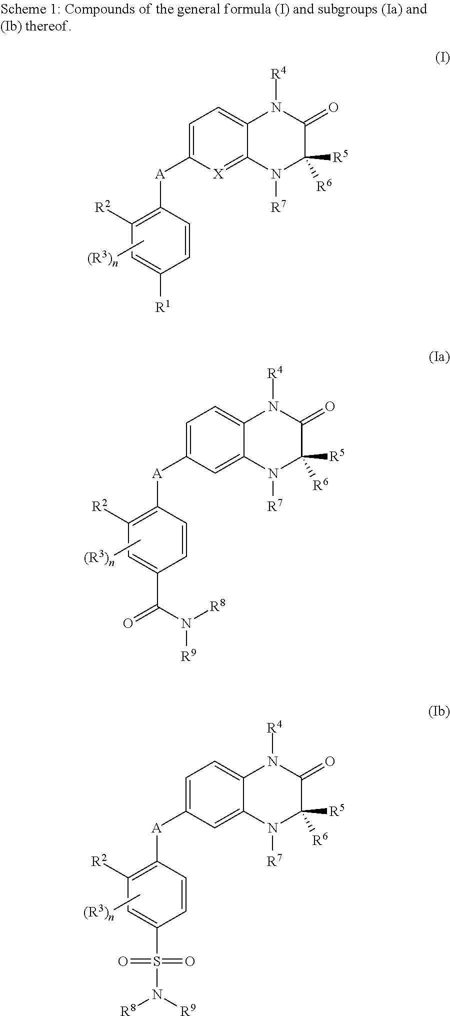

In addition to the synthesis sequences discussed hereinafter, it is also possible, in accordance with the general knowledge of the person skilled in the art in organic chemistry, to take further synthesis routes for the synthesis of inventive compounds of the general formula (I). The sequence of the synthesis steps shown in the schemes which follow is not binding, and synthesis steps from various of the schemes shown hereinafter may optionally be combined to form new sequences. In addition, interconversions of the substituents R², R³, R⁴, R⁵, R⁶, R⁷, R⁸, R⁹ can be performed before or after the synthesis stages shown. Examples of such conversions are the introduction or elimination of protecting groups, reduction or oxidation of functional groups, halogenation, metallation, metal-catalysed coupling reactions, substitution reactions or further reactions known to those skilled in the art. These reactions include conversions which introduce a functional group which enables the further conversion of substituents. Suitable protecting groups and methods for introduction and elimination thereof are known to those skilled in the art (see, for example, T.W. Greene and P.G.M. Wuts in: Protective Groups in Organic Synthesis, 3rd edition, Wiley 1999). In addition, it is possible to combine two or more reaction steps without intermediate workup in a manner known to those skilled in the art (for example in what are called “one-pot” reactions).

Compounds of the general formula (I) and the precursors thereof described hereinafter in which mutually different R⁵ and R⁶ are present are chiral and may occur as enantiomer mixtures, for example racemates, or as pure enantiomers. The enantiomer mixtures mentioned can be separated into the enantiomers by separation methods familiar to those skilled in the art, for example preparative HPLC on a chiral stationary phase.

Dihydroquinoxalinones with a carboxamide group of the formula (Ia) can be obtained as described in Schemes 2, 3 and 4. For this purpose, it is possible to react suitable ortho-fluoronitrobenzene derivatives, for example 4-bromo-2-fluoronitrobenzene ((II); CAS No. 321-23-3), by nucleophilic ipso substitution with amino acids of the structure (III) in which R⁵ and R⁶ are each as defined for the general formula (I) to give compounds of the structure (IV). By selective reduction of the nitro group with a suitable reducing agent and subsequent workup in an acidic medium, the bicyclic compounds of the formula (V) are obtained directly. Suitable reducing agents which may be used are, for example, alkali metal dithionites (J. Heterocyclic Chem. (1992), 29, p. 1859-61, Shafiee et al.), or tin(II) chloride (J. Org. Chem. (1983), 48, p. 2515ff, Xing et al.). The overall reaction sequence of reduction and cyclization has likewise been described (WO2010/116270 A1, L.1.b). For preparation of the compounds (VI) substituted on the basic nitrogen, in which R⁷ is as defined in the general formula (I), the compounds of the formula (V) can be reacted with aldehydes or ketones suitable for the introduction of R⁷ and a reducing agent by a reductive amination known to those skilled in the art. Here, for example, the use of an alkyl- or arylsilane, for example phenylsilane, as the reducing agent is a method which is known to those skilled in the art and gives the intermediate (VI) in adequate yields (Bioorg. Med. Chem. Lett. (2009), 19, p. 688ff; D. V. Smil et al.). The subsequent alkylation to give compounds (VII) can be effected by reaction with R⁴-LG in which R⁴ is as defined in the general formula (I) and LG is a leaving group, preferably iodide, in the presence of a suitable base such as sodium hydride, under conditions known to those skilled in the art.

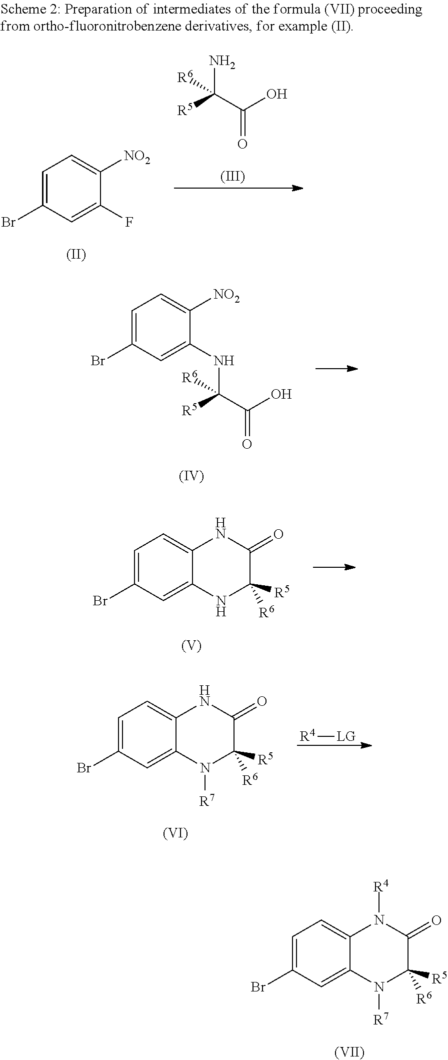

ortho-Fluoronitrobenzene derivatives, for example (II), and amino acids of the formula (III) are known to those skilled in the art and commercially available. An alternative route to intermediates of the formula (V) is shown in Scheme 3. In this case, amino acid esters of the structure (IIIa) in which R⁵ and R⁶ are each as defined for the general formula (I), and in which R^{E} is C₁-C₆-alkyl, are reacted in a reductive amination known to those skilled in the art with aldehydes or ketones suitable for the introduction of R⁷ and a reducing agent, for example sodium triacetoxyborohydride, to form N-substituted amino acid esters of the formula (VIII). These are subsequently reacted with suitable ortho-fluoronitrobenzene derivatives, for example 4-bromo-2-fluoronitrobenzene (II), by nucleophilic ipso substitution in the presence of a suitable base, for example potassium carbonate, in aqueous ethanol to give N,N-disubstituted amino acids of the formula (IX); the ester present in (IIIa) is hydrolysed under these reaction conditions. The N,N-disubstituted amino acids of the formula (IX) can be cyclized under reductive conditions, for example with iron powder in a mixture of methanol and acetic acid, to give the compounds of the formula (V) (Pesticide Science (1999), 55, p. 281ff.; J. W. Lyga et al.), which can then, as discussed in Scheme 2, be converted further to intermediates of the formula (VII).

Amino acid esters of the formula (IIIa) are known to those skilled in the art and many are commercially available.

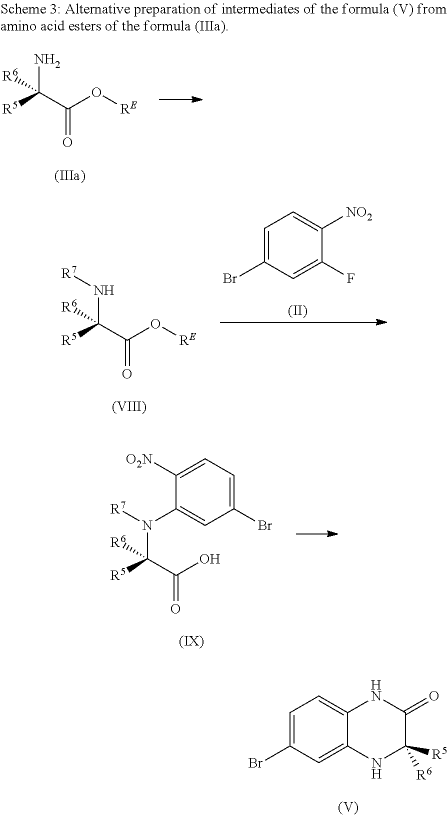

The conversion of compounds of the formula (VII) obtainable as described above, in which R⁴, R⁵, R⁶ and R⁷ are each as defined in the general formula (I), to the ester derivatives (XI) can be effected according to Scheme 4, by reaction with compounds of the formula (X) in which A, R², R³, and n are each as defined in the general formula (I), and in which R^{E} is C₁-C₆-alkyl, in a palladium-catalysed coupling reaction according to Buchwald and Hartwig (see, for example, J. Organomet. Chem. (1999), 576, p. 125 ft). Examples of palladium sources suitable here are palladium(II) acetate or palladium-dba complexes, for example Pd₂(dba)₃ (CAS Nos. 51364-51-3 and 52409-22-0). The conversion depends strongly on the ligands used. The examples adduced in the experimental section could thus be obtained, for example, through the use of racemic BINAP or (±)-BINAP (when A=—NH—; cf. also US2006/009457 A1); when A=—O—, di-tert-butyl(2′,4′,6′-triisopropylbiphenyl-2-yl)phosphine or a structurally similar ligand was advantageously used (Eur. J. Org. Chem. (2010), 34, p. 66651T, C. Schneider et al.).

The subsequent preparation of carboxamides of the general formula (Ia) can be effected by means of hydrolysis of the respective esters of the formula (XI) to give the corresponding carboxylic acids of the formula (XII) by methods known to those skilled in the art. These reactions can preferably be performed using alkali metal hydroxides such as lithium hydroxide, sodium hydroxide or potassium hydroxide in aqueous alcoholic solutions, optionally with the addition of a cyclic ether, for example tetrahydrofuran.

The carboxylic acids (XII) obtained in this way can be converted to the inventive carboxamides of the general formula (Ia) by reaction, for example, with the generally commercially available amines, specified in the working examples, of the formula R⁸R⁹NH in which R⁸ and R⁹ are each as defined for the general formula (I), with additional activation by a method as commonly known to those skilled in the art. Possible methods which should be mentioned here include the use of TBTU, HATU, HBTU, PyBOB or T3P with the addition of a suitable base. The conversion of the carboxylic acids to their amides is described in general terms in reference books such as “Compendium of Organic Synthetic Methods”, volume I-VI (Wiley Interscience) or “The Practice of Peptide Synthesis”, Bodansky (Springer Verlag).

The reaction routes described for Schemes 1 to 4 allow, in the case of the use of an enantiomerically pure amino acid of the formula (III) or of an enantiomerically pure amino acid ester of the formula (IIIa), very substantial suppression of epimerization or racemization of the stereogenic site at the carbon atom bonded to R⁵ and R⁶ on commencement of the sequence. Compounds of the formula (X) are known to those skilled in the art and in many cases commercially available.

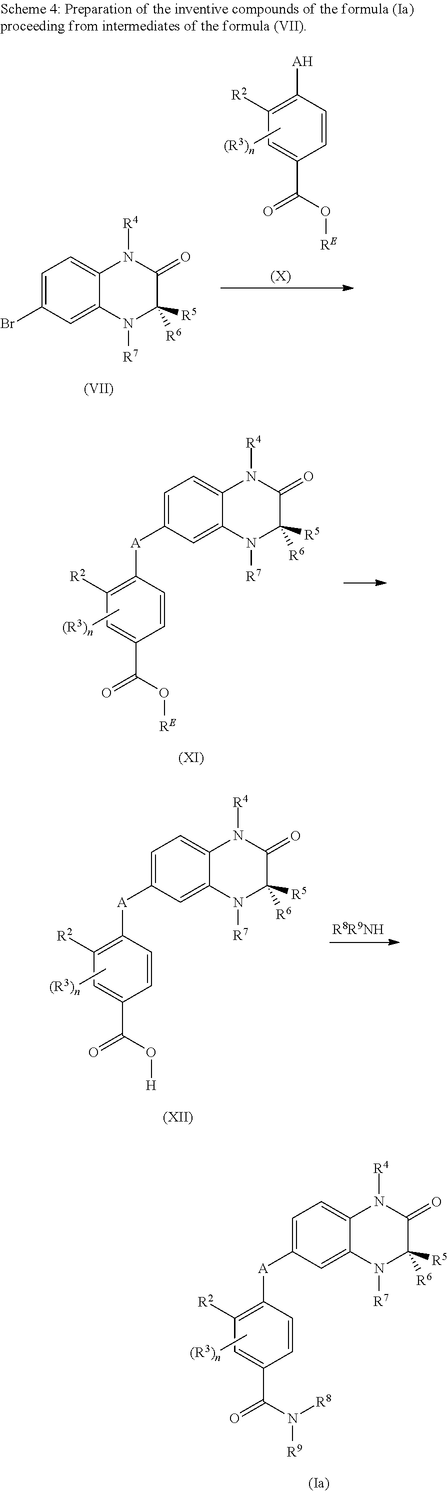

The preparation of the inventive compounds of the formula (Ib) having a sulphonamide group in place of R¹ can be effected according to Scheme 5. In this context, compounds of the formula (VII) can be reacted directly, in an analogous manner to that discussed in Scheme 4 for the conversion of (VII) to (XI), with compounds of the formula (XIII) in which A, R², R³, R⁸, R⁹ and n are each as defined in the general formula (I) in a Palladium-catalysed coupling reaction according to Buchwald and Hartwig to give the inventive compounds of the formula (Ib).

Compounds of the formula (XIII) are known to those skilled in the art and in many cases commercially available.

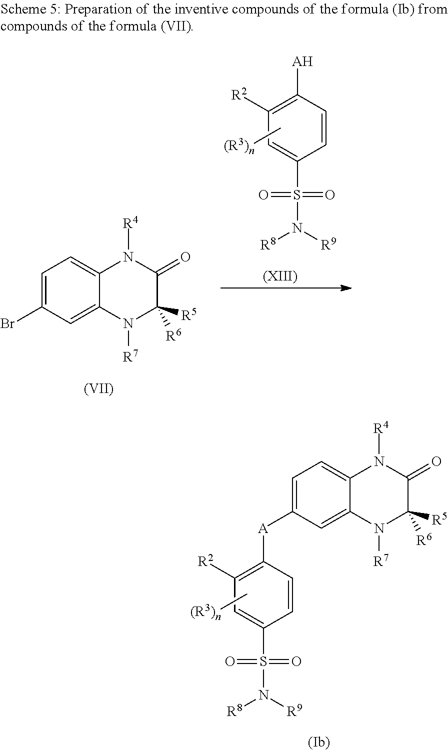

The preparation of intermediates of the formula (VIa) in which R⁷ is optionally substituted phenyl as per the definition of the general formula (I) is described in Scheme 6. 4-Bromo-2-fluoroaniline (XIV; CAS 367-24-8) is reacted with compounds of the formula (XV) in which R⁵ and R⁶ are each as defined for the general formula (I), and in which LG and LG′ are each independently a leaving group, preferably chlorine or bromine, for example 2-bromopropionyl bromide (CAS 563-76-8). This is done by conversion, under conditions known to those skilled in the art, with a suitable solvent such as dichloromethane or THF and with addition of a base such as triethylamine, di-iso-propylethylamine or pyridine. The base can also be used as the solvent. This gives compounds of the formula (XVI). These intermediates (XVI) are reacted with anilines of the formula R⁷—NH₂ in which R⁷ is optionally substituted phenyl as per the definition of the general formula (I) to give compounds of the formula (XVII). This reaction can be effected by reaction in various solvents such as toluene or acetonitrile and with addition of a base such as potassium carbonate, di-iso-propylethylamine or triethylamine at elevated temperature (Org. Lett. (2008), 10, p. 2905ff, S. P. Marsden et al.). Dihydroquinoxalinones of the formula (VIa) in which R⁷ is optionally substituted phenyl as per the definition of the general formula (I) are obtained by cyclizing the compounds of the formula (XVII) in the presence of a suitable base such as triethylamine, di-iso-propylethylamine or potassium carbonate under elevated temperature (in this regard, see also WO2010/96426 A2, Example 16). From these intermediates of the formula (VIa), it is possible according to Schemes 2, 4 and 5 to prepare the corresponding inventive compounds of the formula (I) in which R⁷ is optionally substituted phenyl as per the definition of the general formula (I). This gives the compounds of the formula (I) as racemates if R⁵ and R⁶ are different from one another. These can optionally be separated into the enantiomers by separation methods familiar to those skilled in the art, for example preparative HPLC on a chiral stationary phase.

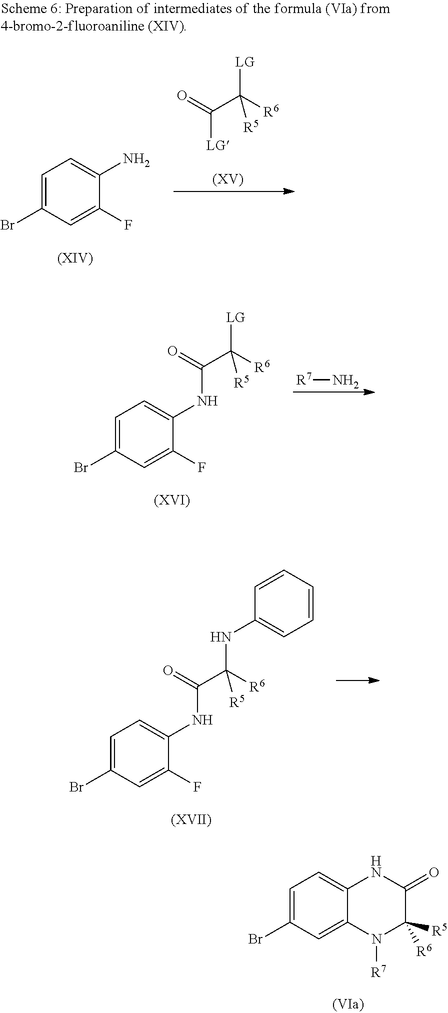

The present invention likewise provides the intermediates of the general formula (XI)

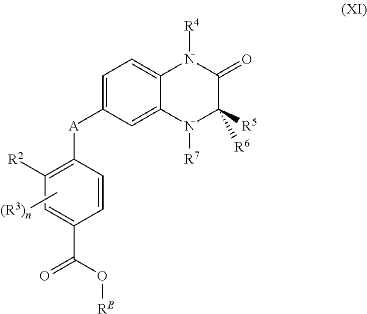

in which A, R², R³, R⁴, R⁵, R⁶, R⁷ and n are each as defined in the general formula (I) and R^{E} is C₁-C₆-alkyl, which can preferably be used for preparation of the inventive compounds of the general formula (I).

The present invention further provides the intermediates of the general formula (XII)

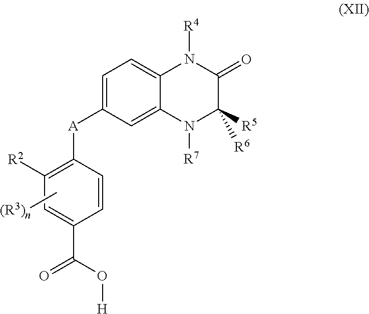

in which A, R², R³, R⁴, R⁵, R⁶, R⁷ and n are each as defined in the general formula (I), and which can likewise preferably be used for preparation of the inventive compounds of the general formula (I).

Especially valuable intermediates for preparation of the inventive compounds are the following compounds:
4-{[(3R)-4-cyclopentyl-1,3-dimethyl-2-oxo-1,2,3,4-tetrahydroquinoxalin-6-yl]amino}benzoic acid methyl ester;
4-{[(3R)-4-cyclopentyl-1,3-dimethyl-2-oxo-1,2,3,4-tetrahydroquinoxalin-6-yl]amino}benzoic acid;
4-{[(3R)-4-benzyl-1,3-dimethyl-2-oxo-1,2,3,4-tetrahydroquinoxalin-6-yl]amino}benzoic acid methyl ester;
4-{[(3R)-4-benzyl-1,3-dimethyl-2-oxo-1,2,3,4-tetrahydroquinoxalin-6-yl]amino}benzoic acid;
4-{[(3R)-4-cyclopentyl-1,3-dimethyl-2-oxo-1,2,3,4-tetrahydroquinoxalin-6-yl]oxy}benzoic acid ethyl ester;
4-{[(3R)-4-cyclopentyl-1,3-dimethyl-2-oxo-1,2,3,4-tetrahydroquinoxalin-6-yl]oxy}benzoic acid;
4-{[(3R)-4-(4-methoxybenzyl)-1,3-dimethyl-2-oxo-1,2,3,4-tetrahydroquinoxalin-6-yl]amino}benzoic acid methyl ester;
4-{[(3R)-4-(4-methoxybenzyl)-1,3-dimethyl-2-oxo-1,2,3,4-tetrahydroquinoxalin-6-yl]amino}benzoic acid;
4-{[(3R)-4-cycloheptyl-1,3-dimethyl-2-oxo-1,2,3,4-tetrahydroquinoxalin-6-yl]amino}benzoic acid methyl ester;
4-{[(3R)-4-cycloheptyl-1,3-dimethyl-2-oxo-1,2,3,4-tetrahydroquinoxalin-6-yl]amino}benzoic acid;
4-{[(3R)-1,3-dimethyl-2-oxo-4-(tetrahydro-2H-pyran-4-yl)-1,2,3,4-tetrahydroquinoxalin-6-yl]amino}benzoic acid methyl ester;
4-{[(3R)-1,3-dimethyl-2-oxo-4-(tetrahydro-2H-pyran-4-yl)-1,2,3,4-tetrahydroquinoxalin-6-yl]amino}benzoic acid;
4-{[(3R)-1,3-dimethyl-2-oxo-4-(tetrahydro-2H-pyran-4-yl)-1,2,3,4-tetrahydroquinoxalin-6-yl]amino}-3-methoxybenzoic acid methyl ester;
4-{[(3R)-1,3-dimethyl-2-oxo-4-(tetrahydro-2H-pyran-4-yl)-1,2,3,4-tetrahydroquinoxalin-6-yl]amino}-3-methoxybenzoic acid;
4-{[4-(2,6-difluorobenzyl)-1,3-dimethyl-2-oxo-1,2,3,4-tetrahydroquinoxalin-6-yl]amino}benzoic acid ethyl ester;
4-{[4-(2,6-difluorobenzyl)-1,3-dimethyl-2-oxo-1,2,3,4-tetrahydroquinoxalin-6-yl]amino}benzoic acid.

### WORKING EXAMPLES

The examples which follow illustrate the preparation of the inventive compounds, without restricting the invention to these examples.

Firstly, the preparation of the intermediates is described, which are preferably used ultimately for preparation of the inventive compounds.

IUPAC names were created with the aid of the nomenclature software ACD Name batch, Version 12.01, from Advanced Chemical Development, Inc., and adapted if required, for example to German-language nomenclature.

### Preparation of the Intermediates

### Intermediate 1

### N-(5-bromo-2-nitrophenyl)-D-alanine

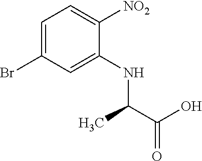 [SMILES: C[C@@H](Nc1cc(Br)ccc1[N+](=O)[O-])C(=O)O]

A solution of 13.57 g of 4-bromo-2-fluoronitrobenzene, 5.49 g of D-alanine and 10.66 g of potassium carbonate in 150 ml of ethanol and 60 ml of water was heated under reflux for 6 hours.

After cooling to room temperature, the pH was acidified with 1 M hydrochloric acid and the product formed was filtered off as a precipitate. This gave 17.36 g of N-(5-bromo-2-nitrophenyl)-D-alanine.

Alternative Batch on a Larger Scale:

A solution of 35.6 g of 4-bromo-2-fluoronitrobenzene (CAS No. 321-23-3), 14.4 g of D-alanine and 27.95 g of potassium carbonate in 395 ml of ethanol and 175 ml of water was heated under reflux for 6 hours. After cooling to room temperature, the reaction mixture was acidified by addition of 1 N hydrochloric acid and the product formed was filtered off as a precipitate. This gave 45.56 g of N-(5-bromo-2-nitrophenyl)-D-alanine.

¹H NMR (400 MHz, CDCl₃): δ=1.46 (d, 3H); 4.52-4.62 (m, 1H); 6.89 (dd, 1H); 7.22 (d, 1H); 8.01 (d, 1H); 8.38 (d, 1H).

### Intermediate 2

### (3R)-6-bromo-3-methyl-3,4-dihydroquinoxalin-2(1H)-one

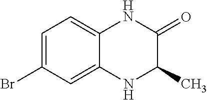

A solution of 5.19 g of Intermediate 1 and 4.96 g of potassium carbonate in 150 ml of water was admixed dropwise with a solution of 9.37 g of sodium dithionite in 50 ml of water at RT over 30 min. After a further 30 min at RT, the pH was acidified with 2 M hydrochloric acid and the mixture was stirred briefly. The mixture was neutralized with potassium carbonate and extracted with dichloromethane. The organic phase was dried over sodium sulphate and concentrated completely under reduced pressure. This gave 1.88 g of (3R)-6-bromo-3-methyl-3,4-dihydroquinoxalin-2(1H)-one.

Alternative Batch on a Larger Scale:

A solution of 45.56 g of Intermediate 1 in 158 ml of methanol and 158 ml of acetic acid was admixed with 30.8 g of iron powder and heated under reflux for 7 hours. The suspension was filtered through kieselguhr and the solution was freed of methanol under reduced pressure. The residue was diluted with dichloromethane and extracted with sodium hydroxide solution. The aqueous phase was extracted twice more with dichloromethane and the combined organic phases were dried over sodium sulphate. The solvent was removed completely under reduced pressure and the residue was purified by chromatography on silica gel (hexane/ethyl acetate gradient). This gave 17.2 g of (3R)-6-bromo-3-methyl-3,4-dihydroquinoxalin-2(1H)-one.

¹H NMR (400 MHz, CDCl₃): δ=1.47 (d, 3H); 3.90 (bs, 1H); 4.03 (q, 1H); 6.62 (d, 1H); 6.82 (d, 1H); 6.87 (dd, 1H); 8.68 (bs, 1H).

### Intermediate 3

### (3R)-6-bromo-4-cyclopentyl-3-methyl-3,4-dihydroquinoxalin-2(1H)-one

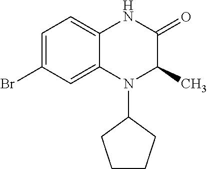 [SMILES: C[C@@H]1C(=O)Nc2ccc(Br)cc2N1C1CCCC1]

A solution of 1.36 g of Intermediate 2, 1.42 g of cyclopentanone, 1.83 g of phenylsilane and 1.71 g of dibutyltin dichloride in 40 ml of THF was stirred at RT for 72 hours. The solution was concentrated completely under reduced pressure and purified by chromatography on silica gel (dichloromethane/methanol 9:1). This gave 2.11 g of (3R)-6-bromo-4-cyclopentyl-3-methyl-3,4-dihydroquinoxalin-2(1H)-one.

¹H NMR (400 MHz, CDCl₃): δ=1.16 (d, 3H); 1.57-1.85 (m, 6H); 1.95-2.08 (m, 2H); 3.82 (qi, 1H); 4.12 (q, 1H); 6.67 (d, 1H); 6.92 (dd, 1H); 6.98 (d, 1H); 9.05 (bs, 1H).

### Intermediate 4

### (3R)-6-bromo-4-cyclopentyl-1,3-dimethyl-3,4-dihydroquinoxalin-2(1H)-one

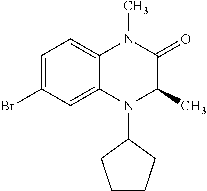 [SMILES: C[C@@H]1C(=O)N(C)c2ccc(Br)cc2N1C1CCCC1]

A solution of 2.11 g of Intermediate 3 and 1.45 g of methyl iodide in 40 ml of DMF was admixed at 0° C. with 409 mg of sodium hydride (60% in white oil) in portions. After a further 30 min at 0° C., saturated ammonium chloride solution was added and the mixture was diluted with dichloromethane. The organic phase was removed and dried over sodium sulphate. The solvent was removed under reduced pressure and the residue was purified by chromatography on silica gel (dichloromethane/methanol 95:5). This gave 2.24 g of (3R)-6-bromo-4-cyclopentyl-1,3-dimethyl-3,4-dihydroquinoxalin-2(1H)-one.

¹H NMR (400 MHz, CDCl₃): δ=1.06 (d, 3H); 1.55-1.84 (2 m, 6H); 1.97-2.09 (m, 2H); 3.34 (s, 3H); 3.77 (qi, 1H); 4.18 (q, 1H); 6.79 (d, 1H); 6.94 (d, 1H); 6.98 (dd, 1H).

### Intermediate 5

### 4-{[(3R)-4-cyclopentyl-1,3-dimethyl-2-oxo-1,2,3,4-tetrahydroquinoxalin-6-yl]amino}benzoic acid methyl ester

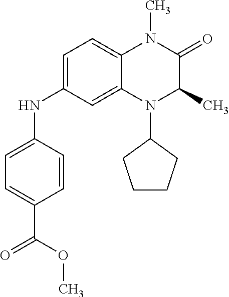 [SMILES: COC(=O)c1ccc(Nc2ccc3c(c2)N(C2CCCC2)[C@H](C)C(=O)N3C)cc1]

A suspension of 496 mg of Intermediate 4, 463 mg of methyl 4-aminobenzoate, 68.9 mg of palladium(II) acetate, 2 g of caesium carbonate and 191 mg of (+)-BINAP in 20 ml of toluene was stirred under an argon atmosphere at 110° C. for 6 hours. The reaction solution was filtered, the residue was washed with ethyl acetate, and the combined organic phases were extracted with water and concentrated completely under reduced pressure. The residue was purified by chromatography on silica gel (hexane/ethyl acetate gradient). This gave 388 mg of 4-{[(3R)-4-cyclopentyl-1,3-dimethyl-2-oxo-1,2,3,4-tetrahydroquinoxalin-6-yl]amino}benzoic acid methyl ester.

¹H NMR (400 MHz, CDCl₃): δ=1.10 (d, 3H); 1.54-1.84 (m, 6H); 1.93-2.06 (m, 2H); 3.38 (s, 3H); 3.72 (qi, 1H); 3.88 (s, 3H); 4.20 (q, 1H); 5.97 (bs, 1H); 6.66-6.75 (m, 2H); 6.91 (d, 1H); 6.94 (d, 2H); 7.92 (d, 2H).

### Intermediate 6

### 4-{[(3R)-4-cyclopentyl-1,3-dimethyl-2-oxo-1,2,3,4-tetrahydroquinoxalin-6-yl]amino}benzoic acid

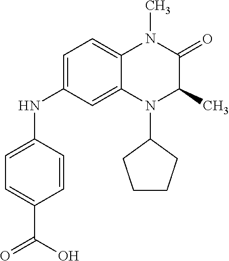 [SMILES: C[C@@H]1C(=O)N(C)c2ccc(Nc3ccc(C(=O)O)cc3)cc2N1C1CCCC1]

A solution of 378 mg of Intermediate 5 and 9.6 ml of 1N of lithium hydroxide solution in 3 ml of THF and 13 ml of methanol was stirred at 50° C. for 14 hours. After cooling to RT by adding 1N hydrochloric acid, the solution was adjusted to pH<7 and extracted with chloroform/methanol 9:1. The combined organic phases were dried over sodium sulphate and the solvent was removed completely under reduced pressure. This gave 452 mg of the title compound as a crude product, which was used without further purification.

UPLC-MS: Rt=1.11 min (M⁺+1=380)

Instrument: Waters Acquity UPLC-MS SQD; column: Acquity UPLC BEH C18 1.7 50×2.1 mm; eluent A: water+0.1% by vol. of formic acid (99%), eluent B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 7

### (3R)-4-benzyl-6-bromo-3-methyl-3,4-dihydroquinoxalin-2(1H)-one

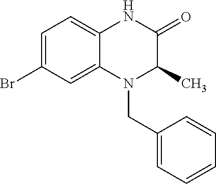 [SMILES: C[C@@H]1C(=O)Nc2ccc(Br)cc2N1Cc1ccccc1]

In analogy to the preparation of Intermediate 3, (3R)-4-benzyl-6-bromo-3-methyl-3,4-dihydroquinoxalin-2(1H)-one was prepared proceeding from 1.58 g of Intermediate 2, 2.09 g of benzaldehyde, 2.13 g of phenylsilane and 1.99 g of dibutyltin dichloride in 40 ml of THF. After chromatography on silica gel (hexane/ethyl acetate gradient), 2.15 g of (3R)-4-benzyl-6-bromo-3-methyl-3,4-dihydroquinoxalin-2(1H)-one were obtained.

¹H NMR (400 MHz, CDCl₃): δ=1.20 (d, 3H); 3.93 (q, 1H); 4.17 (d, 1H); 4.57 (d, 1H); 6.65 (d, 1H); 6.84 (d, 1H); 6.89 (dd, 1H); 7.29-7.39 (m, 5H); 8.79 (bs, 1H).

### Intermediate 8

### (3R)-4-benzyl-6-bromo-1,3-dimethyl-3,4-dihydroquinoxalin-2(1H)-one

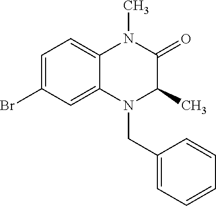 [SMILES: C[C@@H]1C(=O)N(C)c2ccc(Br)cc2N1Cc1ccccc1]

In analogy to the preparation of Intermediate 4, (3R)-4-benzyl-6-bromo-1,3-dimethyl-3,4-dihydroquinoxalin-2(1H)-one was prepared proceeding from 2.15 g of Intermediate 7, 389 mg of sodium hydride (60% in white oil) and 1.38 g of methyl iodide in 40 ml of DMF. After chromatography on silica gel (hexane/ethyl acetate gradient), 2.12 g of (3R)-4-benzyl-6-bromo-1,3-dimethyl-3,4-dihydroquinoxalin-2(1H)-one were obtained.

¹H NMR (400 MHz, CDCl₃): δ=1.10 (d, 3H); 3.36 (s, 3H); 3.95 (q, 1H); 4.11 (d, 1H); 4.53 (d, 1H); 6.80 (d, 1H); 6.84 (d, 1H); 6.98 (dd, 1H); 7.28-7.39 (m, 5H).

### Intermediate 9

### 4-{[(3R)-4-benzyl-1,3-dimethyl-2-oxo-1,2,3,4-tetrahydroquinoxalin-6-yl]amino}benzoic acid methyl ester

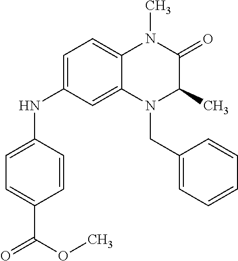 [SMILES: COC(=O)c1ccc(Nc2ccc3c(c2)N(Cc2ccccc2)[C@H](C)C(=O)N3C)cc1]

In analogy to the preparation of Intermediate 5, 4-{[(3R)-4-benzyl-1,3-dimethyl-2-oxo-1,2,3,4-tetrahydroquinoxalin-6-yl]amino}benzoic acid methyl ester was prepared proceeding from 1.0 g of Intermediate 8, 657 mg of methyl 4-aminobenzoate, 130 mg of palladium(II) acetate, 3.78 g of caesium carbonate and 361 mg of (±)-BINAP in 40 ml of toluene after stirring at 110° C. under an argon atmosphere for 6 hours. After chromatography on silica gel (hexane/ethyl acetate gradient), 805 mg of 4-{[(3R)-4-benzyl-1,3-dimethyl-2-oxo-1,2,3,4-tetrahydroquinoxalin-6-yl]amino}benzoic acid methyl ester were obtained.

¹H NMR (400 MHz, CDCl₃): δ=1.17 (d, 3H); 3.41 (s, 3H); 3.87 (s, 3H); 4.07 (q, 1H); 4.18 (d, 1H); 4.46 (d, 1H); 5.89 (bs, 1H); 6.47 (d, 1H); 6.60 (dd, 1H); 6.68 (d, 2H); 6.90 (d, 1H); 7.29-7.39 (m, 5H); 7.78 (d, 2H).

### Intermediate 10

### 4-{[(3R)-4-benzyl-1,3-dimethyl-2-oxo-1,2,3,4-tetrahydroquinoxalin-6-yl]amino}benzoic acid

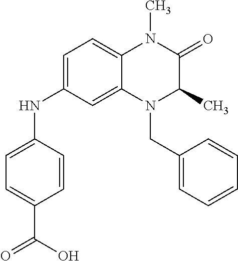 [SMILES: C[C@@H]1C(=O)N(C)c2ccc(Nc3ccc(C(=O)O)cc3)cc2N1Cc1ccccc1]

In analogy to the preparation of Intermediate 6, 4-{[(3R)-4-benzyl-1,3-dimethyl-2-oxo-1,2,3,4-tetrahydroquinoxalin-6-yl]amino}benzoic acid was prepared proceeding from 805 mg of Intermediate 9 and 19.4 ml of 1N aqueous lithium hydroxide solution in 5 ml of THF and 20 ml of methanol. This gave 685 mg of 4-{[(3R)-4-benzyl-1,3-dimethyl-2-oxo-1,2,3,4-tetrahydroquinoxalin-6-yl]amino}benzoic acid, which was used in the next stage without further purification.

UPLC-MS: Rt=0.66 min (M⁺+1=402)

Instrument: Waters Acquity UPLC-MS SQD; column: Acquity UPLC BEH C18 1.7×50×2.1 mm; eluent A: water+0.2% by vol. of NH₃ (32%), eluent B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 11

### 4-{[(3R)-4-cyclopentyl-1,3-dimethyl-2-oxo-1,2,3,4-tetrahydroquinoxalin-6-yl]oxy}benzoic acid ethyl ester

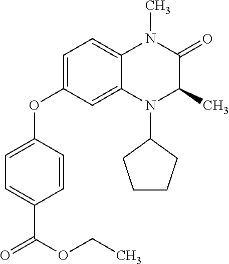 [SMILES: CCOC(=O)c1ccc(Oc2ccc3c(c2)N(C2CCCC2)[C@H](C)C(=O)N3C)cc1]

A solution of 366 mg of Intermediate 4, 376 mg of ethyl 4-hydroxybenzoate, 51 mg of palladium(II) acetate, 721 mg of potassium phosphate and 96 mg of di-tert-butyl(2′,4′,6′-triisopropylbiphenyl-2-yl)phosphine in 6 ml of toluene was stirred at 110° C. in an argon atmosphere for 72 hours. After cooling, the mixture was filtered through kieselguhr and concentrated completely under reduced pressure. The residue was purified twice by chromatography on silica gel (1st eluent: dichloromethane/methanol 98:2; 2nd eluent: hexane/ethyl acetate gradient). This gave 55 mg of 4-{[(3R)-4-cyclopentyl-1,3-dimethyl-2-oxo-1,2,3,4-tetrahydroquinoxalin-6-yl]oxy}benzoic acid ethyl ester.

¹H NMR (400 MHz, CDCl₃): δ=1.09 (d, 3H); 1.38 (t, 3H); 1.51-1.83 (m, 6H); 1.90-2.07 (m, 2H); 3.38 (s, 3H); 3.70 (qi, 1H); 4.20 (q, 1H); 4.36 (q, 2H); 6.52-6.60 (m, 2H); 6.91 (d, 1H); 6.98 (d, 2H); 8.01 (d, 2H).

### Intermediate 12

### 4-{[(3R)-4-cyclopentyl-1,3-dimethyl-2-oxo-1,2,3,4-tetrahydroquinoxalin-6-yl]oxy}benzoic acid

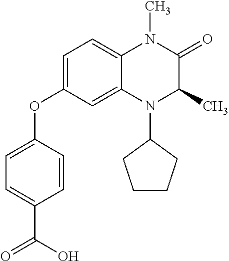 [SMILES: C[C@@H]1C(=O)N(C)c2ccc(Oc3ccc(C(=O)O)cc3)cc2N1C1CCCC1]

In analogy to the preparation of Intermediate 6, 4-{[(3R)-4-cyclopentyl-1,3-dimethyl-2-oxo-1,2,3,4-tetrahydroquinoxalin-6-yl]oxy}benzoic acid was prepared proceeding from 55 mg of Intermediate 11 and 1.4 ml of 1N lithium hydroxide solution in 0.4 ml of THF and 1.9 ml of methanol. This gave 54 mg of 4-{[(3R)-4-cyclopentyl-1,3-dimethyl-2-oxo-1,2,3,4-tetrahydroquinoxalin-6-yl]oxy}benzoic acid, which was used in the next stage without further purification.

UPLC-MS: Rt=1.25 min (M⁺+1=381)

Instrument: Waters Acquity UPLC-MS SQD; column: Acquity UPLC BEH C18 1.7×50×2.1 mm; eluent A: water+0.1% by vol. of formic acid (99%), eluent B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 13

### (3R)-6-bromo-4-(4-methoxybenzyl)-3-methyl-3,4-dihydroquinoxalin-2(1H)-one

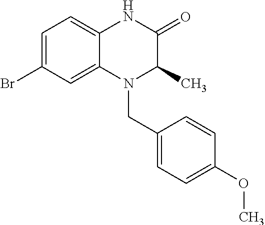 [SMILES: COc1ccc(CN2c3cc(Br)ccc3NC(=O)[C@H]2C)cc1]

In analogy to the preparation of Intermediate 3, (3R)-6-bromo-4-(4-methoxybenzyl)-3-methyl-3,4-dihydroquinoxalin-2(1H)-one was prepared proceeding from 1.53 g of Intermediate 2, 2.59 g of 4-methoxybenzaldehyde, 2.06 g of phenylsilane and 1.93 g of dibutyltin hydride. After chromatography on silica gel (hexane/ethyl acetate 3:2), 2.06 g of the title compound were obtained.

¹H NMR (400 MHz, CDCl₃): δ=1.17 (d, 3H); 3.82 (s, 3H); 3.90 (q, 1H); 4.09 (d, 1H); 4.51 (d, 1H); 6.65 (d, 1H); 6.85-6.95 (m, 4H); 7.24 (d, 2H); 9.00 (bs, 1H).

### Intermediate 14

### (3R)-6-bromo-4-(4-methoxybenzyl)-1,3-dimethyl-3,4-dihydroquinoxalin-2(1H)-one

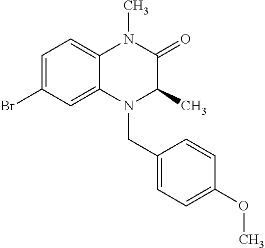 [SMILES: COc1ccc(CN2c3cc(Br)ccc3N(C)C(=O)[C@H]2C)cc1]

In analogy to the preparation of Intermediate 4, (3R)-6-bromo-4-(4-methoxybenzyl)-1,3-dimethyl-3,4-dihydroquinoxalin-2(1H)-one was prepared proceeding from 2.03 g of Intermediate 13, 1.2 g of methyl iodide and 337 mg of sodium hydride (60% in oil). After chromatography on silica gel, (hexane/ethyl acetate gradient), 1.34 g of the title compound were obtained.

¹H NMR (400 MHz, DMSO-d6): δ=0.99 (d, 3H); 3.26 (s, 3H); 3.74 (s, 3H); 3.90 (q, 1H); 4.15 (d, 1H); 4.50 (d, 1H); 6.87 (m, 1H); 6.92 (d, 2H); 6.99 (m, 2H); 7.27 (d, 2H).

### Intermediate 15

### 4-{[(3R)-4-(4-methoxybenzyl)-1,3-dimethyl-2-oxo-1,2,3,4-tetrahydroquinoxalin-6-yl]amino}benzoic acid methyl ester

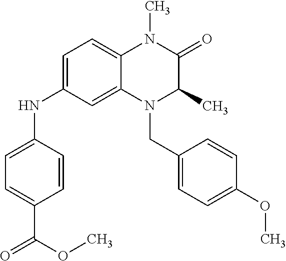 [SMILES: COC(=O)c1ccc(Nc2ccc3c(c2)N(Cc2ccc(OC)cc2)[C@H](C)C(=O)N3C)cc1]

In analogy to the preparation of Intermediate 5, 4-{[(3R)-4-(4-methoxybenzyl)-1,3-dimethyl-2-oxo-1,2,3,4-tetrahydroquinoxalin-6-yl]amino}benzoic acid methyl ester was prepared proceeding from 600 mg of Intermediate 14, 483 mg of methyl 4-aminobenzoate, 36 mg of palladium(II) acetate, 1.56 g of caesium carbonate and 100 mg of (±)-BINAP in 36 ml of toluene after stirring at 110° C. under an argon atmosphere for 17 hours. After chromatography on silica gel (hexane/ethyl acetate gradient), 760 mg of 4-{[(3R)-4-(4-methoxybenzyl)-1,3-dimethyl-2-oxo-1,2,3,4-tetrahydroquinoxalin-6-yl]amino}benzoic acid methyl ester were obtained.

UPLC-MS: Rt=1.27 min (M⁺+1=446)

Instrument: Waters Acquity UPLC-MS SQD; column: Acquity UPLC BEH C18 1.7×50×2.1 mm; eluent A: water+0.1% by vol. of formic acid (99%), eluent B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 16

### 4-{[(3R)-4-(4-methoxybenzyl)-1,3-dimethyl-2-oxo-1,2,3,4-tetrahydroquinoxalin-6-yl]amino}benzoic acid

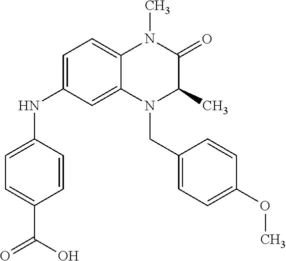 [SMILES: COc1ccc(CN2c3cc(Nc4ccc(C(=O)O)cc4)ccc3N(C)C(=O)[C@H]2C)cc1]

In analogy to the preparation of Intermediate 6, 4-{[(3R)-4-(4-methoxybenzyl)-1,3-dimethyl-2-oxo-1,2,3,4-tetrahydroquinoxalin-6-yl]amino}benzoic acid was prepared proceeding from 760 mg of Intermediate 15 and 17 ml of 1N lithium hydroxide solution in 5 ml of THF and 20 ml of methanol. This gave 900 mg of 4-{[(3R)-4-(4-methoxybenzyl)-1,3-dimethyl-2-oxo-1,2,3,4-tetrahydroquinoxalin-6-yl]amino}benzoic acid, which was used in the next stage without further purification.

¹H NMR (400 MHz, DMSO-d6): δ=1.02 (d, 3H); 3.29 (s, 3H); 3.77 (s, 3H); 4.01 (q, 1H); 4.24 (d, 1H); 4.39 (d, 1H); 6.45 (d, 1H); 6.57 (dd, 1H); 6.66 (d, 2H); 6.92 (d, 2H); 7.00 (d, 1H); 7.25 (d, 2H); 7.60 (d, 2H); 8.52 (s, 1H); 12.19 (bs, 1H).

### Intermediate 17

### (3R)-6-bromo-4-cycloheptyl-3-methyl-3,4-dihydroquinoxalin-2(1H)-one

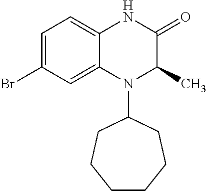 [SMILES: C[C@@H]1C(=O)Nc2ccc(Br)cc2N1C1CCCCCC1]

In analogy to the preparation of Intermediate 3, (3R)-6-bromo-4-cycloheptyl-3-methyl-3,4-dihydroquinoxalin-2(1H)-one was prepared proceeding from 1.55 g of Intermediate 2, 2.16 g of cycloheptanone, 2.09 g of phenylsilane and 2.93 g of dibutyltin hydride. After chromatography on silica gel (hexane/ethyl acetate 3:2), 336 mg of the title compound were obtained.

¹H NMR (400 MHz, CDCl₃): δ=1.17 (d, 3H); 1.27 (t, 1H); 1.35-1.87 (m, 10H); 2.01-2.13 (m, 1H); 3.43-3.57 (m, 1H); 4.06-4.18 (m, 1H); 6.64 (d, 1H); 6.84-6.93 (m, 2H); 8.72 (bs, 1H).

### Intermediate 18

### (3R)-6-bromo-4-cycloheptyl-1,3-dimethyl-3,4-dihydroquinoxalin-2(1H)-one

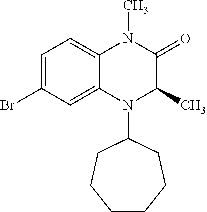 [SMILES: C[C@@H]1C(=O)N(C)c2ccc(Br)cc2N1C1CCCCCC1]

In analogy to the preparation of Intermediate 4, (3R)-6-bromo-4-cycloheptyl-1,3-dimethyl-3,4-dihydroquinoxalin-2(1H)-one was prepared proceeding from 336 mg of Intermediate 17, 148 mg of methyl iodide and 42 mg of sodium hydride (60% in oil). After chromatography on silica gel (hexane/ethyl acetate 3:2), 240 mg of the title compound were obtained.

¹H NMR (400 MHz, CDCl₃): δ=1.09 (d, 3H); 1.38-1.50 (m, 1H); 1.50-1.86 (m, 10H); 2.02-2.10 (m, 1H); 3.34 (s, 3H); 3.45-3.55 (m, 1H); 4.18 (q, 1H); 6.78 (d, 1H); 6.88 (d, 1H); 6.94 (dd, 1H).

### Intermediate 19

### 4-{[(3R)-4-cycloheptyl-1,3-dimethyl-2-oxo-1,2,3,4-tetrahydroquinoxalin-6-yl]amino}benzoic acid methyl ester

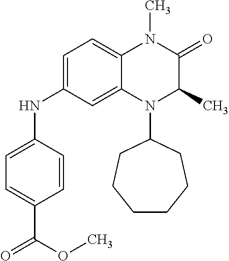 [SMILES: COC(=O)c1ccc(Nc2ccc3c(c2)N(C2CCCCCC2)[C@H](C)C(=O)N3C)cc1]

In analogy to the preparation of Intermediate 5, 4-{[(3R)-4-benzyl-1,3-dimethyl-2-oxo-1,2,3,4-tetrahydroquinoxalin-6-yl]amino}benzoic acid methyl ester was prepared proceeding from 190 mg of Intermediate 18, 123 mg of methyl 4-aminobenzoate, 24 mg of palladium(II) acetate, 529 mg of caesium carbonate and 67 mg of (+)-BINAP in 8 ml of toluene after stirring at 120° C. under an argon atmosphere in a closed vessel for 3 hours. After chromatography on silica gel (hexane/ethyl acetate 3:2), 164 mg of 4-{[(3R)-4-benzyl-1,3-dimethyl-2-oxo-1,2,3,4-tetrahydroquinoxalin-6-yl]amino}benzoic acid methyl ester were obtained.

¹H NMR (400 MHz, CDCl₃): δ=1.13 (d, 3H); 1.36-1.90 (m, 11H); 1.99-2.08 (m, 1H); 3.37 (s, 3H); 3.88 (s, 3H); 3.47 (tt, 1H); 4.20 (q, 1H); 6.06 (s, 1H); 6.60 (d, 1H); 6.67 (dd, 1H); 6.89 (d, 1H); 6.96 (d, 2H); 7.91 (d, 2H).

### Intermediate 20

### 4-{[(3R)-4-cycloheptyl-1,3-dimethyl-2-oxo-1,2,3,4-tetrahydroquinoxalin-6-yl]amino}benzoic acid

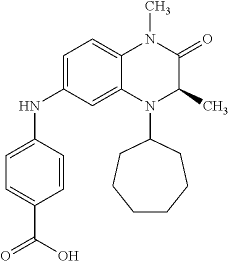 [SMILES: C[C@@H]1C(=O)N(C)c2ccc(Nc3ccc(C(=O)O)cc3)cc2N1C1CCCCCC1]

In analogy to the preparation of Intermediate 6, 4-{[(3R)-4-cycloheptyl-1,3-dimethyl-2-oxo-1,2,3,4-tetrahydroquinoxalin-6-yl]amino}benzoic acid was prepared proceeding from 164 mg of Intermediate 19 and 3.8 ml of lithium hydroxide solution (1M) in 1 ml of THF and 4 ml of methanol. This gave, in quantitative yield, 4-{[(3R)-4-cycloheptyl-1,3-dimethyl-2-oxo-1,2,3,4-tetrahydroquinoxalin-6-yl]amino}benzoic acid, which was used in the next stage without further purification.

UPLC-MS: Rt=0.73 min (M⁺+1=408)

Instrument: Waters Acquity UPLC-MS SQD; column: Acquity UPLC BEH C18 1.7×50×2.1 mm; eluent A: water+0.1% by vol. of ammonia (99%), eluent B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 21

### (3R)-6-bromo-3-methyl-4-(tetrahydro-2H-pyran-4-yl)-3,4-dihydroquinoxalin-2(1H)-one

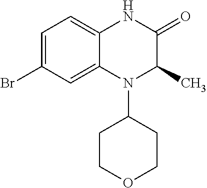 [SMILES: C[C@@H]1C(=O)Nc2ccc(Br)cc2N1C1CCOCC1]

In analogy to the preparation of Intermediate 3, (3R)-6-bromo-3-methyl-4-(tetrahydro-2H-pyran-4-yl)-3,4-dihydroquinoxalin-2(1H)-one was prepared proceeding from 1.54 g of Intermediate 2, 1.92 g of tetrahydro-4H-pyran-4-one, 2.07 g of phenylsilane and 1.94 g of dibutyltin hydride. After chromatography on silica gel (hexane/ethyl acetate gradient), 1.97 g of the title compound were obtained.

¹H NMR (400 MHz, CDCl₃): δ=1.18 (d, 3H); 1.62-1.71 (m, 1H); 1.76-1.92 (m, 2H); 1.92-2.00 (m, 1H); 3.41-3.56 (m, 2H); 3.62 (tt, 1H); 4.00-4.14 (m, 3H); 6.71 (d, 1H); 6.94 (dd, 1H); 6.98 (d, 1H); 9.5 (bs, 1H).

### Intermediate 22

### (3R)-6-bromo-1,3-dimethyl-4-(tetrahydro-2H-pyran-4-yl)-3,4-dihydroquinoxalin-2(1H)-one

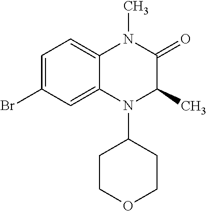 [SMILES: C[C@@H]1C(=O)N(C)c2ccc(Br)cc2N1C1CCOCC1]

In analogy to the preparation of Intermediate 4, (3R)-6-bromo-1,3-dimethyl-4-(tetrahydro-2H-pyran-4-yl)-3,4-dihydroquinoxalin-2(1H)-one was prepared proceeding from 1.97 g of Intermediate 21, 1.29 g of methyl iodide and 363 mg of sodium hydride (60% in oil). After chromatography on silica gel (hexane/ethyl acetate 2:3), 1.54 g of the title compound were obtained.

¹H NMR (400 MHz, CDCl₃): δ=1.10 (d, 3H); 1.58-1.72 (m, 1H); 1.77-2.00 (m, 3H); 3.35 (s, 3H); 3.40-3.68 (m, 3H); 3.99-4.20 (m, 3H); 6.82 (d, 1H); 6.98 (d, 1H); 7.01 (dd, 1H).

### Intermediate 23

### 4-{[(3R)-1,3-dimethyl-2-oxo-4-(tetrahydro-2H-pyran-4-yl)-1,2,3,4-tetrahydroquinoxalin-6-yl]amino}benzoic acid methyl ester

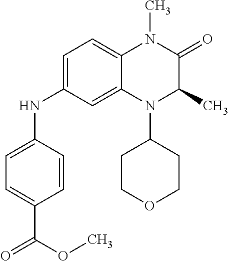 [SMILES: COC(=O)c1ccc(Nc2ccc3c(c2)N(C2CCOCC2)[C@H](C)C(=O)N3C)cc1]

In analogy to the preparation of Intermediate 5, 4-{[(3R)-1,3-dimethyl-2-oxo-4-(tetrahydro-2H-pyran-4-yl)-1,2,3,4-tetrahydroquinoxalin-6-yl]amino}benzoic acid methyl ester was prepared proceeding from 707 mg of Intermediate 22, 630 mg of methyl 4-aminobenzoate, 47 mg of palladium(II) acetate, 2.04 g of caesium carbonate and 130 mg of (+)-BINAP in 15 ml of toluene after stirring at 110° C. under an argon atmosphere in a closed vessel for 8 hours. After chromatography on silica gel (hexane/ethyl acetate 2:3), 677 mg of 4-{[(3R)-1,3-dimethyl-2-oxo-4-(tetrahydro-2H-pyran-4-yl)-1,2,3,4-tetrahydroquinoxalin-6-yl]amino}benzoic acid methyl ester were obtained.

¹H NMR (400 MHz, CDCl₃): δ=1.13 (d, 3H); 1.66-1.76 (m, 1H); 1.77-1.98 (m, 3H); 3.38 (s, 3H); 3.40-3.64 (m, 3H); 3.88 (s, 3H); 3.99-4.12 (m, 2H); 4.15 (q, 1H); 5.97 (s, 1H); 6.69 (d, 1H); 6.76 (dd, 1H); 6.90-6.98 (m, 3H); 7.92 (d, 2H).

### Intermediate 24

### 4-{[(3R)-1,3-dimethyl-2-oxo-4-(tetrahydro-2H-pyran-4-yl)-1,2,3,4-tetrahydroquinoxalin-6-yl]amino}benzoic acid

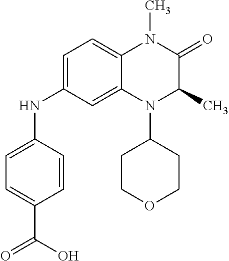 [SMILES: C[C@@H]1C(=O)N(C)c2ccc(Nc3ccc(C(=O)O)cc3)cc2N1C1CCOCC1]

In analogy to the preparation of Intermediate 6, 4-{[(3R)-1,3-dimethyl-2-oxo-4-(tetrahydro-2H-pyran-4-yl)-1,2,3,4-tetrahydroquinoxalin-6-yl]amino}benzoic acid was prepared proceeding from 677 mg of Intermediate 23 and 16.5 ml of lithium hydroxide solution (1N) in 4 ml of THF and 17 ml of methanol. This gave, in quantitative yield, 4-{[(3R)-1,3-dimethyl-2-oxo-4-(tetrahydro-2H-pyran-4-yl)-1,2,3,4-tetrahydroquinoxalin-6-yl]amino}benzoic acid, which was used in the next stage without further purification.

UPLC-MS: Rt=0.54 min (M⁺+1=396)

Instrument: Waters Acquity UPLC-MS SQD; column: Acquity UPLC BEH C18 1.7×50×2.1 mm; eluent A: water+0.1% by vol. of ammonia (99%), eluent B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 25

### 4-{[(3R)-1,3-dimethyl-2-oxo-4-(tetrahydro-2H-pyran-4-yl)-1,2,3,4-tetrahydroquinoxalin-6-yl]amino}-3-methoxybenzoic acid methyl ester

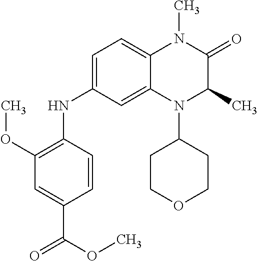 [SMILES: COC(=O)c1ccc(Nc2ccc3c(c2)N(C2CCOCC2)[C@H](C)C(=O)N3C)c(OC)c1]

In analogy to the preparation of Intermediate 5, 4-{[(3R)-1,3-dimethyl-2-oxo-4-(tetrahydro-2H-pyran-4-yl)-1,2,3,4-tetrahydroquinoxalin-6-yl]amino}-3-methoxybenzoic acid methyl ester was prepared proceeding from 2 g of Intermediate 22, 2.03 g of methyl 4-amino-3-methoxybenzoate, 126 mg of palladium(II) acetate, 5.48 g of caesium carbonate and 349 mg of (+)-BINAP in 125 ml of toluene after stirring at 120° C. under an argon atmosphere in a closed vessel for 2 hours. After chromatography on silica gel (hexane/ethyl acetate 3:2), 1.2 g of 4-{[(3R)-1,3-dimethyl-2-oxo-4-(tetrahydro-2H-pyran-4-yl)-1,2,3,4-tetrahydroquinoxalin-6-yl]amino}-3-methoxybenzoic acid methyl ester were obtained.

¹H NMR (400 MHz, CDCl₃): δ=1.13 (d, 3H); 1.71 (bd, 1H); 1.75-1.98 (m, 3H); 3.38 (s, 3H); 3.40-3.51 (m, 2H); 3.58 (tt, 1H); 3.89 (s, 3H); 3.98 (s, 3H); 4.00-4.11 (m, 2H); 4.15 (q, 1H); 6.46 (s, 1H); 6.72 (d, 1H); 6.81 (dd, 1H); 6.94 (d, 1H); 7.11 (d, 1H); 7.54 (s, 1H); 7.60 (dd, 1H).

### Intermediate 26

### 4-{[(3R)-1,3-dimethyl-2-oxo-4-(tetrahydro-2H-pyran-4-yl)-1,2,3,4-tetrahydroquinoxalin-6-yl]amino}-3-methoxybenzoic acid

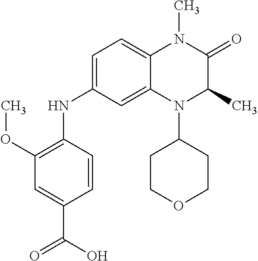 [SMILES: COc1cc(C(=O)O)ccc1Nc1ccc2c(c1)N(C1CCOCC1)[C@H](C)C(=O)N2C]

In analogy to the preparation of Intermediate 6, 4-{[(3R)-1,3-dimethyl-2-oxo-4-(tetrahydro-2H-pyran-4-yl)-1,2,3,4-tetrahydroquinoxalin-6-yl]amino}-3-methoxybenzoic acid was prepared proceeding from 300 mg of Intermediate 25 and 6.5 ml of lithium hydroxide solution (1M) in 2 ml of THF and 16 ml of methanol. This gave 270 mg of 4-{[(3R)-1,3-dimethyl-2-oxo-4-(tetrahydro-2H-pyran-4-yl)-1,2,3,4-tetrahydroquinoxalin-6-yl]amino}-3-methoxybenzoic acid.

¹H NMR (400 MHz, DMSO-d6): δ=0.98 (d, 3H); 1.60 (bd, 1H); 1.63-1.84 (m, 2H); 1.89 (bd, 1H); 3.25 (s, 3H); 3.35-3.47 (m, 2H); 3.62 (tt, 1H); 3.85-3.98 (m+s, 5H); 4.08 (q, 1H); 6.79 (dd, 1H); 6.86 (d, 1H); 7.00 (d, 1H); 7.13 (d, 1H); 7.42 (d, 1H); 7.46 (dd, 1H); 7.71 (s, 1H); 12.20 (bs, 1H).

### Intermediate 27

### N-(2,6-difluorobenzyl)alanine methyl ester

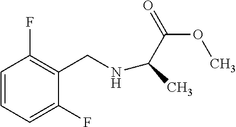 [SMILES: COC(=O)[C@@H](C)NCc1c(F)cccc1F]

A solution of 3.35 g of D-alanine methyl ester and 3.3 ml of triethylamine in 100 ml of dichloromethane was admixed with 2.9 g of 2,6-difluorobenzaldehyde and stirred for 30 min. To this were added 8.5 g of sodium triacetoxyborohydride, and then 2.3 ml of acetic acid were added cautiously at RT. The mixture was stirred for 16 hours, then diluted with dichloromethane and added cautiously to saturated sodium hydrogencarbonate solution. The organic phase was removed, dried over sodium sulphate and freed from the solvent under reduced pressure. This gave 4.7 g of the title compound, which was used without further purification.

UPLC-MS: Rt=1.02 min (M⁺+1=230)

Instrument: Waters Acquity UPLC-MS SQD; column: Acquity UPLC BEH C18 1.7×50×2.1 mm; eluent A: water+0.1% by vol. of ammonia (99%), eluent B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 28

### N-(5-bromo-2-nitrophenyl)-N-(2,6-difluorobenzyl)alanine

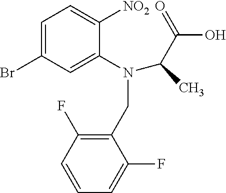 [SMILES: C[C@H](C(=O)O)N(Cc1c(F)cccc1F)c1cc(Br)ccc1[N+](=O)[O-]]

A solution of 2.1 g of Intermediate 27, 1.83 g of 4-bromo-2-fluoronitrobenzene and 1.39 g of potassium carbonate in 20 ml of ethanol and 8 ml of water was stirred at 100° C. in a closed vessel for 6 hours. The mixture was left to stir at RT for a further 56 hours and diluted with water. The pH of the solution was adjusted to <7 with 1N hydrochloric acid and the precipitate was filtered off with suction. This gave 4.7 g of the title compound as a crude product, which was used without further purification.

UPLC-MS: Rt=1.02 min (M⁺+1=415/417)

Instrument: Waters Acquity UPLC-MS SQD; column: Acquity UPLC BEH C18 1.7×50×2.1 mm; eluent A: water+0.1% by volume of formic acid (99%), eluent B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 29

### 6-bromo-4-(2,6-difluorobenzyl)-3-methyl-3,4-dihydroquinoxalin-2(1H)-one

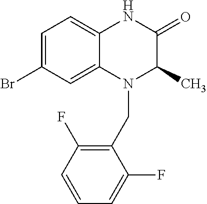 [SMILES: C[C@@H]1C(=O)Nc2ccc(Br)cc2N1Cc1c(F)cccc1F]

4.6 g of Intermediate 28 in 24 ml of methanol and 24 ml of acetic acid were admixed with 2.2 g of iron powder and stirred at 105° C. in a closed vessel for 2 hours. The mixture was filtered and the solution was concentrated completely under reduced pressure. The residue was purified by chromatography on silica gel (dichloromethane/methanol gradient). This gave 970 mg of the title compound.

¹H NMR (400 MHz, DMSO-d6): δ=1.08 (d, 3H); 3.74 (q, 1H); 4.29 (d, 1H); 4.67 (d, 1H); 6.73 (d, 1H); 6.89 (dd, 1H); 7.04 (d, 1H); 7.16 (t, 2H); 7.45 (qi, 1H); 10.52 (bs, 1H).

### Intermediate 30

### 6-bromo-4-(2,6-difluorobenzyl)-1,3-dimethyl-3,4-dihydroquinoxalin-2(1H)-one

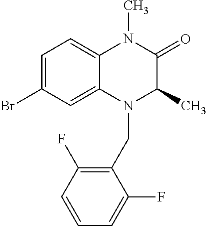 [SMILES: C[C@@H]1C(=O)N(C)c2ccc(Br)cc2N1Cc1c(F)cccc1F]

In analogy to the preparation of Intermediate 4, 6-bromo-4-(2,6-difluorobenzyl)-1,3-dimethyl-3,4-dihydroquinoxalin-2(1H)-one was prepared proceeding from 970 mg of Intermediate 29, 552 mg of methyl iodide and 169 mg of sodium hydride (60% in oil). This gave 1.15 g of the title compound as a crude product.

UPLC-MS: Rt=1.36 min (M⁺+1=381/383)

Instrument: Waters Acquity UPLC-MS SQD; column: Acquity UPLC BEH C18 1.7×50×2.1 mm; eluent A: water+0.1% by volume of formic acid (99%), eluent B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 31

### 4-{[4-(2,6-difluorobenzyl)-1,3-dimethyl-2-oxo-1,2,3,4-tetrahydroquinoxalin-6-yl]amino}benzoic acid ethyl ester

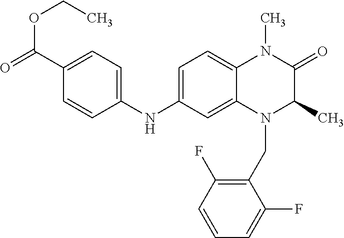 [SMILES: CCOC(=O)c1ccc(Nc2ccc3c(c2)N(Cc2c(F)cccc2F)[C@H](C)C(=O)N3C)cc1]

In analogy to the preparation of Intermediate 5, 4-{[4-(2,6-difluorobenzyl)-1,3-dimethyl-2-oxo-1,2,3,4-tetrahydroquinoxalin-6-yl]amino}benzoic acid ethyl ester was prepared proceeding from 161 mg of Intermediate 30, 131 mg of ethyl 4-aminobenzoate, 18 mg of palladium(II) acetate, 646 mg of caesium carbonate and 49 mg of (+)-BINAP in 4 ml of toluene after stirring at 120° C. under an argon atmosphere in a closed vessel for 3 hours. After chromatography on silica gel (hexane/ethyl acetate 3:2), 165 mg of 4-{[4-(2,6-difluorobenzyl)-1,3-dimethyl-2-oxo-1,2,3,4-tetrahydroquinoxalin-6-yl]amino}benzoic acid ethyl ester were obtained.

UPLC-MS: Rt=1.35 min (M⁺+1=466)

Instrument: Waters Acquity UPLC-MS SQD; column: Acquity UPLC BEH C18 1.7×50×2.1 mm; eluent A: water+0.1% by vol. of formic acid (99%), eluent B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Intermediate 32

### 4-{[4-(2,6-difluorobenzyl)-1,3-dimethyl-2-oxo-1,2,3,4-tetrahydroquinoxalin-6-yl]amino}benzoic acid

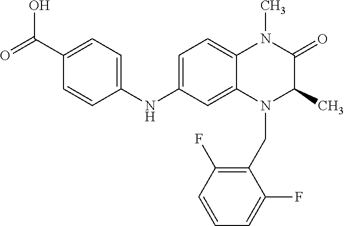 [SMILES: C[C@@H]1C(=O)N(C)c2ccc(Nc3ccc(C(=O)O)cc3)cc2N1Cc1c(F)cccc1F]

In analogy to the preparation of Intermediate 6, 4-{[4-(2,6-difluorobenzyl)-1,3-dimethyl-2-oxo-1,2,3,4-tetrahydroquinoxalin-6-yl]amino}benzoic acid was prepared proceeding from 165 mg of Intermediate 31 and 0.88 ml of sodium hydroxide solution (2N) in 4 ml of ethanol. This gave 4-{[4-(2,6-difluorobenzyl)-1,3-dimethyl-2-oxo-1,2,3,4-tetrahydroquinoxalin-6-yl]amino}benzoic acid quantitatively, which was used in the next stage without further purification.

UPLC-MS: Rt=1.08 min (M⁺+1=438)

Instrument: Waters Acquity UPLC-MS SQD; column: Acquity UPLC BEH C18 1.7×50×2.1 mm; eluent A: water+0.1% by vol. of ammonia (99%), eluent B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Preparation of the Inventive Compounds

### Example 1

### N-cyclopentyl-4-{[(3R)-4-cyclopentyl-1,3-dimethyl-2-oxo-1,2,3,4-tetrahydroquinoxalin-6-yl]amino}benzamide

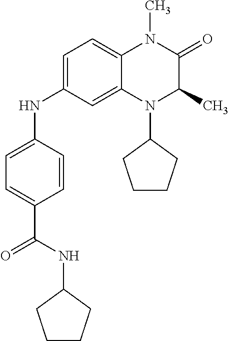 [SMILES: C[C@@H]1C(=O)N(C)c2ccc(Nc3ccc(C(=O)NC4CCCC4)cc3)cc2N1C1CCCC1]

A solution of 121 mg of Intermediate 6, 61 mg of cyclopentylamine, 103 mg of N,N-diisopropylethylamine and 304 mg of HATU in 3 ml of DMF was stirred at RT for 15 hours. The reaction solution was filtered and concentrated under reduced pressure, and the residue was purified by RP-HPLC chromatography (column: X-Bridge C18, 5 μm 100×30 mm, mobile phase: acetonitrile/water (0.2% by vol. of ammonia) gradient). This gave 57 mg of N-cyclopentyl-4-{[(3R)-4-cyclopentyl-1,3-dimethyl-2-oxo-1,2,3,4-tetrahydroquinoxalin-6-yl]amino}benzamide.

¹H NMR (400 MHz, DMSO-d6): δ=0.91 (d, 3H); 1.42-1.71 (m, 12H); 1.77-2.00 (m, 4H); 3.21 (s, 3H); 3.68 (qi, 1H); 4.02 (q, 1H); 4.16 (qi, 1H); 6.59 (d, 1H); 6.63 (dd, 1H); 6.92-6.99 (m, 3H); 7.69 (d, 2H); 7.89 (d, 1H); 8.34 (bs, 1H).

### Example 2

### 4-{[(3R)-4-cyclopentyl-1,3-dimethyl-2-oxo-1,2,3,4-tetrahydroquinoxalin-6-yl]amino}-N-cyclopropylbenzamide

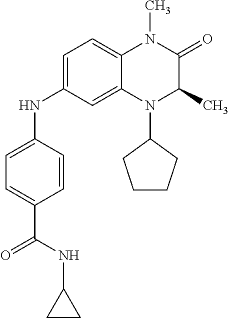 [SMILES: C[C@@H]1C(=O)N(C)c2ccc(Nc3ccc(C(=O)NC4CC4)cc3)cc2N1C1CCCC1]

In analogy to the preparation of Example 1, 4-{[(3R)-4-cyclopentyl-1,3-dimethyl-2-oxo-1,2,3,4-tetrahydroquinoxalin-6-yl]amino}-N-cyclopropylbenzamide was prepared proceeding from 121 mg of Intermediate 6, 46 mg of cyclopropylamine, 103 mg of N,N-diisopropylethylamine and 304 mg of HATU in 3 ml of DMF. After RP-HPLC chromatography (column: X-Bridge C18 5 μm 100×30 mm, mobile phase: acetonitrile/water (0.2% by vol. of ammonia) gradient), 74 mg of 4-{[(3R)-4-cyclopentyl-1,3-dimethyl-2-oxo-1,2,3,4-tetrahydroquinoxalin-6-yl]amino}-N-cyclopropylbenzamide were obtained.

¹H NMR (400 MHz, DMSO-d6): δ=0.47-0.52 (m, 2H); 0.59-0.65 (m, 2H); 0.91 (d, 3H); 1.45-1.72 (m, 6H); 1.87-2.00 (m, 2H); 2.72-2.81 (m, 1H); 3.21 (s, 3H); 3.68 (qi, 1H); 4.01 (q, 1H); 6.59 (d, 1H); 6.63 (dd, 1H); 6.92-6.97 (m, 3H); 7.65 (d, 2H); 8.06 (d, 1H); 8.35 (bs, 1H).

### Example 3

### 4-{[(3R)-4-cyclopentyl-1,3-dimethyl-2-oxo-1,2,3,4-tetrahydroquinoxalin-6-yl]amino}-N,N-dimethylbenzenesulphonamide

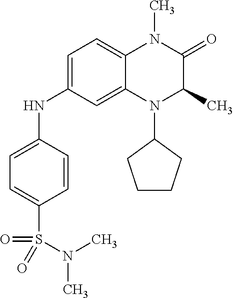 [SMILES: C[C@@H]1C(=O)N(C)c2ccc(Nc3ccc(S(=O)(=O)N(C)C)cc3)cc2N1C1CCCC1]

A suspension of 105 mg of Intermediate 4, 130 mg of 4-amino-N,N-dimethylbenzenesulphonamide (CAS 1709-59-7), 15 mg of palladium(II) acetate, 318 mg of caesium carbonate and 41 mg of (+)-BINAP in 3 ml of toluene was stirred at 110° C. under an argon atmosphere for 3 hours. The reaction solution was filtered, the residue was washed with ethyl acetate, and the combined organic phases were extracted with water and concentrated completely under reduced pressure. The residue was purified by RP-HPLC chromatography (column: X-Bridge C18 5 μm 100×30 mm, mobile phase: acetonitrile/water (0.2% by vol. of ammonia) gradient). This gave 57 mg of 4-{[(3R)-4-cyclopentyl-1,3-dimethyl-2-oxo-1,2,3,4-tetrahydroquinoxalin-6-yl]amino}-N,N-dimethylbenzenesulphonamide.

¹H NMR (400 MHz, CDCl₃): δ=1.08 (d, 3H); 1.53-1.82 (m, 6H); 1.92-2.06 (m, 2H); 2.68 (s, 6H); 3.37 (s, 3H); 3.72 (qi, 1H); 4.19 (q, 1H); 6.13 (bs, 1H); 6.64-6.75 (m, 2H); 6.90 (d, 1H); 6.98 (d, 2H); 7.60 (d, 2H).

### Example 4

### 4-{[(3R)-4-cyclopentyl-1,3-dimethyl-2-oxo-1,2,3,4-tetrahydroquinoxalin-6-yl]amino}-N-(1-methylpiperidin-4-yl)benzamide

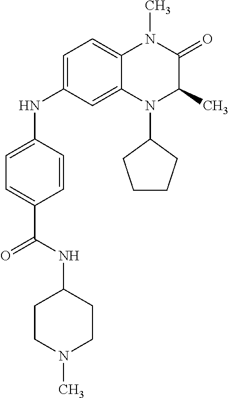 [SMILES: C[C@@H]1C(=O)N(C)c2ccc(Nc3ccc(C(=O)NC4CCN(C)CC4)cc3)cc2N1C1CCCC1]

In analogy to the preparation of Example 1, 4-{[(3R)-4-cyclopentyl-1,3-dimethyl-2-oxo-1,2,3,4-tetrahydroquinoxalin-6-yl]amino}-N-(1-methylpiperidin-4-yl)benzamide was prepared proceeding from 121 mg of Intermediate 6, 91 mg of 4-amino-1-methylpiperidine, 103 mg of N,N-diisopropylethylamine and 304 mg of HATU in 3 ml of DMF. After RP-HPLC chromatography (column: X-Bridge C18 5 μm 100×30 mm, mobile phase: acetonitrile/water (0.2% by vol. of ammonia) gradient), 73 mg of 4-{[(3R)-4-cyclopentyl-1,3-dimethyl-2-oxo-1,2,3,4-tetrahydroquinoxalin-6-yl]amino}-N-(1-methylpiperidin-4-yl)benzamide were obtained.

¹H NMR (400 MHz, CDCl₃): δ=1.03 (d, 3H); 1.49-1.78 (m, 6H); 1.88-2.03 (m, 2H); 2.10-2.20 (m, 2H); 2.31 (q, 2H); 2.74 (s, 3H); 2.84-2.96 (m, 2H); 3.32 (s, 3H); 3.41-3.51 (m, 2H); 3.67 (qi, 1H); 4.15 (q, 1H); 4.19-4.30 (m, 1H); 6.40 (bs, 1H); 6.60-6.67 (m, 2H); 6.83 (d, 1H); 6.95 (d, 2H); 7.05 (d, 1H); 7.72 (d, 2H).

### Example 5

### 4-{[(3R)-4-cyclopentyl-1,3-dimethyl-2-oxo-1,2,3,4-tetrahydroquinoxalin-6-yl]oxy}-N-cyclopropylbenzamide

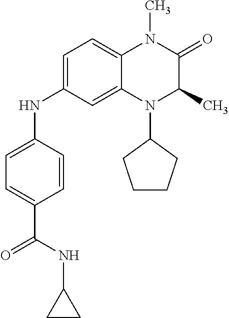 [SMILES: C[C@@H]1C(=O)N(C)c2ccc(Nc3ccc(C(=O)NC4CC4)cc3)cc2N1C1CCCC1]

In analogy to the preparation of Example 1, 4-{[(3R)-4-cyclopentyl-1,3-dimethyl-2-oxo-1,2,3,4-tetrahydroquinoxalin-6-yl]oxy}-N-cyclopropylbenzamide was prepared proceeding from 51 mg of Intermediate 12, 19 mg of cyclopropylamine, 44 mg of N,N-diisopropylethylamine and 128 mg of HATU in 2 ml of DMF. After RP-HPLC chromatography (column: X-Bridge C18 5 μm 100×30 mm, mobile phase: acetonitrile/water (0.2% by vol. of ammonia) gradient), 33 mg of 4-{[(3R)-4-cyclopentyl-1,3-dimethyl-2-oxo-1,2,3,4-tetrahydroquinoxalin-6-yl]oxy}-N-cyclopropylbenzamide were obtained.

¹H NMR (400 MHz, CDCl₃): δ=0.57-0.64 (m, 2H); 0.82-0.90 (m, 2H); 1.07 (d, 3H); 1.49-1.81 (m, 6H); 1.87-2.02 (m, 2H); 2.83-2.94 (m, 1H); 3.36 (s, 3H); 3.68 (qi, 1H); 4.18 (q, 1H); 6.26 (bs, 1H); 6.49-6.56 (m, 2H); 6.89 (d, 1H); 6.97 (d, 2H); 7.71 (d, 2H).

### Example 6

### (3R)-4-cyclopentyl-1,3-dimethyl-6-{[4-(morpholin-4-ylcarbonyl)phenyl]amino}-3,4-dihydroquinoxalin-2(1H)-one

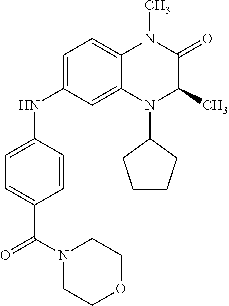 [SMILES: C[C@@H]1C(=O)N(C)c2ccc(Nc3ccc(C(=O)N4CCOCC4)cc3)cc2N1C1CCCC1]

In analogy to the preparation of Example 1, (3R)-4-cyclopentyl-1,3-dimethyl-6-{[4-(morpholin-4-ylcarbonyl)phenyl]amino}-3,4-dihydroquinoxalin-2(1H)-one was prepared proceeding from 93 mg of Intermediate 6, 53 mg of morpholine, 79 mg of N,N-diisopropylethylamine and 233 mg of HATU in 3 ml of DMF. After RP-HPLC chromatography (column: X-Bridge C18 5 μm 100×30 mm, mobile phase: acetonitrile/water (0.2% by vol. of ammonia) gradient), 58 mg of (3R)-4-cyclopentyl-1,3-dimethyl-6-{[4-(morpholin-4-ylcarbonyl)phenyl]amino}-3,4-dihydroquinoxalin-2(1H)-one were obtained.

¹H NMR (400 MHz, CDCl₃): δ=1.07 (d, 3H); 1.52-1.82 (m, 6H); 1.92-2.04 (m, 2H); 3.36 (s, 3H); 3.60-3.77 (m, 9H); 4.17 (q, 1H); 5.89 (bs, 1H); 6.60-6.69 (m, 2H); 6.87 (d, 1H); 6.96 (d, 2H); 7.33 (d, 2H).

### Example 7

### 4-{[(3R)-4-cyclopentyl-1,3-dimethyl-2-oxo-1,2,3,4-tetrahydroquinoxalin-6-yl]amino}-N-isopropylbenzamide

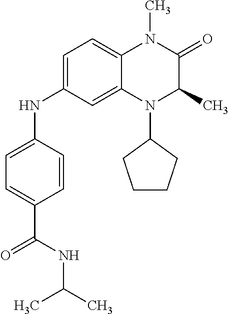 [SMILES: CC(C)NC(=O)c1ccc(Nc2ccc3c(c2)N(C2CCCC2)[C@H](C)C(=O)N3C)cc1]

In analogy to the preparation of Example 1, 4-{[(3R)-4-cyclopentyl-1,3-dimethyl-2-oxo-1,2,3,4-tetrahydroquinoxalin-6-yl]amino}-N-isopropylbenzamide was prepared proceeding from 93 mg of Intermediate 6, 36 mg of isopropylamine, 79 mg of N,N-diisopropylethylamine and 233 mg of HATU in 3 ml of DMF. After RP-HPLC chromatography (column: X-Bridge C18 5 μm 100×30 mm, mobile phase: acetonitrile/water (0.2% by vol. of ammonia) gradient), 36 mg of 4-{[(3R)-4-cyclopentyl-1,3-dimethyl-2-oxo-1,2,3,4-tetrahydroquinoxalin-6-yl]amino}-N-isopropylbenzamide were obtained.

¹H NMR (400 MHz, CDCl₃): δ=1.06 (d, 3H); 1.24 (d, 6H); 1.50-1.85 (m, 6H); 1.89-2.05 (m, 2H); 3.35 (s, 3H); 3.69 (qi, 1H); 4.17 (q, 1H); 4.21-4.35 (m, 1H); 5.86 (bd, 1H); 6.04 (bs, 1H); 6.60-6.69 (m, 2H); 6.87 (d, 1H); 6.96 (d, 2H); 7.65 (d, 2H).

### Example 8

### 4-{[(3R)-4-cyclopentyl-1,3-dimethyl-2-oxo-1,2,3,4-tetrahydroquinoxalin-6-yl]amino}-N,N-dimethylbenzamide

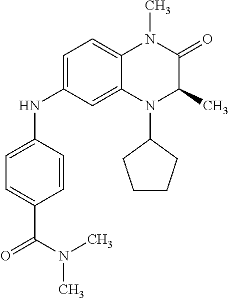 [SMILES: C[C@@H]1C(=O)N(C)c2ccc(Nc3ccc(C(=O)N(C)C)cc3)cc2N1C1CCCC1]

In analogy to the preparation of Example 1, 4-{[(3R)-4-cyclopentyl-1,3-dimethyl-2-oxo-1,2,3,4-tetrahydroquinoxalin-6-yl]amino}-N,N-dimethylbenzamide was prepared proceeding from 93 mg of Intermediate 6, 50 mg of dimethylamine hydrochloride, 79 mg of N,N-diisopropylethylamine and 233 mg of HATU in 3 ml of DMF. After RP-HPLC chromatography (column: X-Bridge C18 5 μm 100×30 mm, mobile phase: acetonitrile/water (0.2% by vol. of ammonia) gradient), 54 mg of 4-{[(3R)-4-cyclopentyl-1,3-dimethyl-2-oxo-1,2,3,4-tetrahydroquinoxalin-6-yl]amino}-N,N-dimethylbenzamide were obtained.

¹H NMR (400 MHz, CDCl₃): δ=1.06 (d, 3H); 1.50-1.83 (m, 6H); 1.89-2.06 (m, 2H); 3.07 (s, 6H); 3.35 (s, 3H); 3.70 (qi, 1H); 4.17 (q, 1H); 5.88 (bs, 1H); 6.59-6.69 (m, 2H); 6.86 (d, 1H); 6.95 (d, 2H); 7.35 (d, 2H).

### Example 9

### 4-{[(3R)-4-benzyl-1,3-dimethyl-2-oxo-1,2,3,4-tetrahydroquinoxalin-6-yl]amino}-N-(oxetan-3-ylmethyl)benzamide

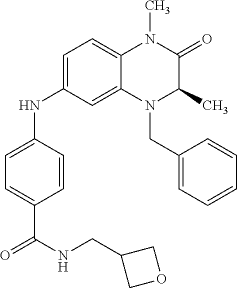 [SMILES: C[C@@H]1C(=O)N(C)c2ccc(Nc3ccc(C(=O)NCC4COC4)cc3)cc2N1Cc1ccccc1]

In analogy to the preparation of Example 1, 4-{[(3R)-4-benzyl-1,3-dimethyl-2-oxo-1,2,3,4-tetrahydroquinoxalin-6-yl]amino}-N-(oxetan-3-ylmethyl)benzamide was prepared proceeding from 113 mg of Intermediate 10, 61 mg of 1-(oxetan-3-yl)methanamine, 91 mg of N,N-diisopropylethylamine and 268 mg of HATU in 3 ml of DMF. After RP-HPLC chromatography (column: X-Bridge C18 5 μm 100×30 mm, mobile phase: acetonitrile/water (0.2% by vol. of ammonia) gradient), 69 mg of 4-{[(3R)-4-benzyl-1,3-dimethyl-2-oxo-1,2,3,4-tetrahydroquinoxalin-6-yl]amino}-N-(oxetan-3-ylmethyl)benzamide were obtained.

¹H NMR (400 MHz, CDCl₃): δ=1.15 (d, 3H); 3.28 (sept, 1H); 3.39 (s, 3H); 3.71 (t, 2H); 4.03 (q, 1H); 4.15 (d, 1H); 4.41-4.52 (m, 3H); 4.82 (t, 2H); 5.95 (bs, 1H); 6.37 (bt, 1H); 6.45 (d, 1H); 6.58 (dd, 1H); 6.72 (d, 2H); 6.88 (d, 1H); 7.27-7.39 (m, 5H); 7.54 (d, 2H).

### Example 10

### 4-{[(3R)-4-benzyl-1,3-dimethyl-2-oxo-1,2,3,4-tetrahydroquinoxalin-6-yl]amino}-N-cyclopropylbenzamide

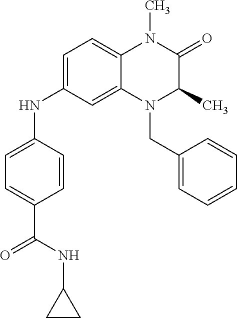 [SMILES: C[C@@H]1C(=O)N(C)c2ccc(Nc3ccc(C(=O)NC4CC4)cc3)cc2N1Cc1ccccc1]

In analogy to the preparation of Example 1, 4-{[(3R)-4-benzyl-1,3-dimethyl-2-oxo-1,2,3,4-tetrahydroquinoxalin-6-yl]amino}-N-cyclopropylbenzamide was prepared proceeding from 113 mg of Intermediate 10, 40 mg of cyclopropylamine, 91 mg of N,N-diisopropylethylamine and 268 mg of HATU in 3 ml of DMF. After RP-HPLC chromatography (column: X-Bridge C18 5 μm 100×30 mm, mobile phase: acetonitrile/water (0.2% by vol. of ammonia) gradient), 72 mg of 4-{[(3R)-4-benzyl-1,3-dimethyl-2-oxo-1,2,3,4-tetrahydroquinoxalin-6-yl]amino}-N-cyclopropylbenzamide were obtained.

¹H NMR (400 MHz, DMSO-d6): δ=0.47-0.52 (m, 2H); 0.59-0.65 (m, 2H); 1.00 (d, 3H); 2.71-2.80 (m, 1H); 3.25 (s, 3H); 3.98 (q, 1H); 4.27 (d, 1H); 4.41 (d, 1H); 6.40 (d, 1H); 6.53 (dd, 1H); 6.67 (d, 2H); 6.95 (d, 1H); 7.24-7.36 (m, 5H); 7.52 (d, 2H); 8.03 (bd, 1H); 8.27 (bs, 1H).

### Example 11

### 4-{[(3R)-4-benzyl-1,3-dimethyl-2-oxo-1,2,3,4-tetrahydroquinoxalin-6-yl]amino}-N-(1-methylpiperidin-4-yl)benzamide

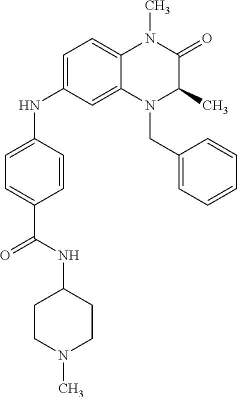 [SMILES: C[C@@H]1C(=O)N(C)c2ccc(Nc3ccc(C(=O)NC4CCN(C)CC4)cc3)cc2N1Cc1ccccc1]

In analogy to the preparation of Example 1, 4-{[(3R)-4-benzyl-1,3-dimethyl-2-oxo-1,2,3,4-tetrahydroquinoxalin-6-yl]amino}-N-(1-methylpiperidin-4-yl)benzamide was prepared proceeding from 113 mg of Intermediate 10, 80 mg of 4-amino-1-methylpiperidine, 91 mg of N,N-diisopropylethylamine and 268 mg of HATU in 3 ml of DMF. After RP-HPLC chromatography (column: X-Bridge C18 5 μm 100×30 mm, mobile phase: acetonitrile/water (0.2% by vol. of ammonia) gradient), 99 mg of 4-{[(3R)-4-benzyl-1,3-dimethyl-2-oxo-1,2,3,4-tetrahydroquinoxalin-6-yl]amino}-N-(1-methylpiperidin-4-yl)benzamide were obtained.

¹H NMR (400 MHz, DMSO-d6): δ=1.00 (d, 3H); 1.45-1.60 (m, 2H); 1.64-1.74 (m, 2H); 1.84-1.95 (m, 2H); 2.12 (s, 3H); 2.68-2.77 (m, 2H); 3.26 (s, 3H); 3.60-3.72 (m, 1H); 3.98 (q, 1H); 4.27 (d, 1H); 4.41 (d, 1H); 6.41 (bs, 1H); 6.54 (d, 1H); 6.69 (d, 2H); 6.95 (d, 1H); 7.23-7.37 (m, 5H); 7.56 (d, 2H); 7.81 (d, 1H); 8.27 (bs, 1H).

### Example 12

### (3R)-4-benzyl-1,3-dimethyl-6-{[4-(morpholin-4-ylcarbonyl)phenyl]amino}-3,4-dihydroquinoxalin-2(1H)-one

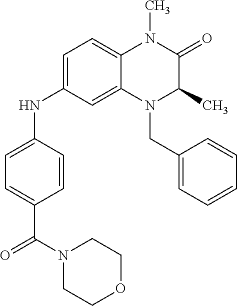 [SMILES: C[C@@H]1C(=O)N(C)c2ccc(Nc3ccc(C(=O)N4CCOCC4)cc3)cc2N1Cc1ccccc1]

In analogy to the preparation of Example 1, (3R)-4-benzyl-1,3-dimethyl-6-{[4-(morpholin-4-ylcarbonyl)phenyl]amino}-3,4-dihydroquinoxalin-2(1H)-one was prepared proceeding from 113 mg of Intermediate 10, 61 mg of morpholine, 91 mg of N,N-diisopropylethylamine and 268 mg of HATU in 3 ml of DMF. After RP-HPLC chromatography (column: X-Bridge C18 5 μm 100×30 mm, mobile phase: acetonitrile/water (0.2% by vol. of ammonia) gradient), 77 mg of (3R)-4-benzyl-1,3-dimethyl-6-{[4-(morpholin-4-ylcarbonyl)phenyl]amino}-3,4-dihydroquinoxalin-2(1H)-one were obtained.

¹H NMR (400 MHz, CDCl₃): δ=1.15 (d, 3H); 3.39 (s, 3H); 3.56-3.80 (m, 8H); 4.04 (q, 1H); 4.16 (d, 1H); 4.45 (d, 1H); 5.88 (bs, 1H); 6.44 (d, 1H); 6.56 (dd, 1H); 6.70 (d, 2H); 6.87 (d, 1H); 7.19 (d, 2H); 7.28-7.39 (m, 5H).

### Example 13

### (3R)-4-benzyl-1,3-dimethyl-6-{[4-(morpholin-4-ylsulphonyl)phenyl]amino}-3,4-dihydroquinoxalin-2(1H)-one

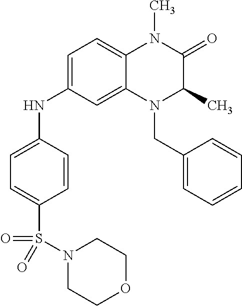 [SMILES: C[C@@H]1C(=O)N(C)c2ccc(Nc3ccc(S(=O)(=O)N4CCOCC4)cc3)cc2N1Cc1ccccc1]

In analogy to the preparation of Example 3, (3R)-4-benzyl-1,3-dimethyl-6-{[4-(morpholin-4-ylsulphonyl)phenyl]amino}-3,4-dihydroquinoxalin-2(1H)-one was prepared proceeding from 78 mg of Intermediate 8, 110 mg of 4-(morpholin-4-ylsulphonyl)aniline (CAS 21626-70-0), 10 mg of palladium(II) acetate, 221 mg of caesium carbonate and 28 mg of (±)-BINAP in 3 ml of toluene after stirring at 110° C. under an argon atmosphere for 3 hours. After RP-HPLC chromatography (column: X-Bridge C18 5 μm 100×30 mm, mobile phase: acetonitrile/water (0.2% by vol. of ammonia) gradient), 67.6 mg of (3R)-4-benzyl-1,3-dimethyl-6-{[4-(morpholin-4-ylsulphonyl)phenyl]amino}-3,4-dihydroquinoxalin-2(1H)-one were obtained.

¹H NMR (400 MHz, CDCl₃): δ=1.19 (d, 3H); 2.97 (t, 4H); 3.42 (s, 3H); 3.75 (t, 4H); 4.08 (q, 1H); 4.21 (d, 1H); 4.47 (d, 1H); 5.99 (bs, 1H); 6.46 (d, 1H); 6.62 (dd, 1H); 6.70 (d, 2H); 6.92 (d, 1H); 7.28-7.39 (m, 5H); 7.43 (d, 2H).

### Example 14

### 4-{[(3R)-4-benzyl-1,3-dimethyl-2-oxo-1,2,3,4-tetrahydroquinoxalin-6-yl]amino}-N,N-dimethylbenzenesulphonamide

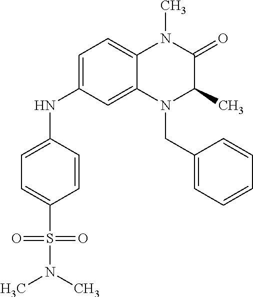 [SMILES: C[C@@H]1C(=O)N(C)c2ccc(Nc3ccc(S(=O)(=O)N(C)C)cc3)cc2N1Cc1ccccc1]

In analogy to the preparation of Example 3, 4-{[(3R)-4-benzyl-1,3-dimethyl-2-oxo-1,2,3,4-tetrahydroquinoxalin-6-yl]amino}-N,N-dimethylbenzenesulphonamide was prepared proceeding from 89 mg of Intermediate 8, 103 mg of 4-amino-N,N-dimethylbenzenesulphonamide (CAS 1709-59-7), 11.5 mg of palladium(II) acetate, 252 mg of caesium carbonate and 32 mg of (±)-BINAP in 3 ml of toluene after stirring at 110° C. under an argon atmosphere for 3 hours. After RP-HPLC chromatography (column: X-Bridge C18 5 μm 100×30 mm, mobile phase: acetonitrile/water (0.2% by vol. of ammonia) gradient), 58 mg of 4-{[(3R)-4-benzyl-1,3-dimethyl-2-oxo-1,2,3,4-tetrahydroquinoxalin-6-yl]amino}-N,N-dimethylbenzenesulphonamide were obtained.

¹H NMR (400 MHz, CDCl₃): δ=1.18 (d, 3H); 2.67 (s, 6H); 3.41 (s, 3H); 4.08 (q, 1H); 4.19 (d, 1H); 4.45 (d, 1H); 6.02 (bs, 1H); 6.44 (d, 1H); 6.60 (d, 1H); 6.69 (d, 2H); 6.91 (d, 1H); 7.32 (m, 5H); 7.44 (d, 2H).

### Example 15

### (3R)-4-benzyl-1,3-dimethyl-6-({4-[(4-methylpiperazin-1-yl)sulphonyl]phenyl}amino)-3,4-dihydroquinoxalin-2(1H)-one

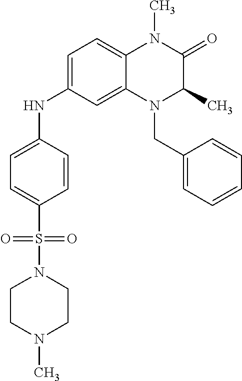 [SMILES: C[C@@H]1C(=O)N(C)c2ccc(Nc3ccc(S(=O)(=O)N4CCN(C)CC4)cc3)cc2N1Cc1ccccc1]

In analogy to the preparation of Example 3, (3R)-4-benzyl-1,3-dimethyl-6-({4-[(4-methylpiperazin-1-yl)sulphonyl]phenyl}amino)-3,4-dihydroquinoxalin-2(1H)-one was prepared proceeding from 83 mg of Intermediate 8, 123 mg of 4-(4-methylpiperazin-1-ylsulphonyl)aniline (CAS 21623-68-7), 11 mg of palladium(II) acetate, 235 mg of caesium carbonate and 30 mg of (±)-BINAP in 3 ml of toluene after stirring at 110° C. under an argon atmosphere for 3 hours. After RP-HPLC chromatography (column: X-Bridge C18 5 μm 100×30 mm, mobile phase: acetonitrile/water (0.2% by vol. of ammonia) gradient), 63 mg of (3R)-4-benzyl-1,3-dimethyl-6-({4-[(4-methylpiperazin-1-yl)sulphonyl]phenyl}amino)-3,4-dihydroquinoxalin-2(1H)-one were obtained.

¹H NMR (400 MHz, CDCl₃): δ=1.19 (d, 3H); 2.29 (s, 3H); 2.45-2.56 (m, 4H); 2.94-3.10 (m, 4H); 3.43 (s, 3H); 4.08 (q, 1H); 4.20 (d, 1H); 4.47 (d, 1H); 5.97 (s, 1H); 6.44 (d, 1H); 6.60 (dd, 1H); 6.67 (d, 2H); 6.92 (d, 1H); 7.29-7.38 (m, 5H); 7.42 (d, 2H).

Tables 1a and 1b

In analogy to the preparation of Example 1, the examples shown in Table 1b were prepared from the intermediates specified in each case and from the amines shown in Table 1a:

TABLE 1a

| Amine | | CAS |
|---|---|---|
| No. | Structure | number |
| 1 | 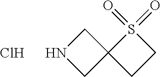 | 1352546- 75-8 |
| 2 | 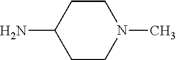 | 41838- 46-4 |
| 3 | 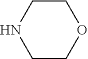 | 110- 91-8 |
| 4 | 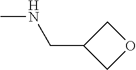 | 6246- 05-5 |
| 5 | 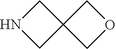 | 1045709- 32-7 |
| 6 | 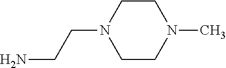 | 934- 98-5 |
| 7 | 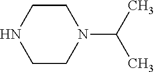 | 4318- 42-7 |
| 8 | 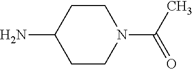 | 160357- 94-8 |
| 9 | 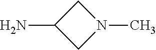 | 959957- 92-7 |
| 10 | 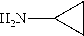 | 765- 30-0 |
| 11 | 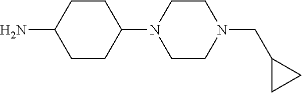 [SMILES: NC1CCC(N2CCN(CC3CC3)CC2)CC1] | 876461- 31-3 |
| 12 | 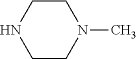 | 109-01-3 |

TABLE 1b

| Ex. | Structure | Name | Intermediate | Analysis |
|---|---|---|---|---|
| 16 | 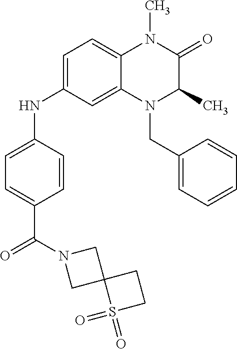 [SMILES: C[C@@H]1C(=O)N(C)c2ccc(Nc3ccc(C(=O)N4CC5(CCS5(=O)=O)C4)cc3)cc2N1Cc1ccccc1] | (3R)-4-benzyl-6-({4- [(1,1-dioxido-1-thia-6- azaspiro[3.3]hept-6- yl)carbonyl]phenyl} amino)-1,3-dimethyl- 3,4-dihydroquinoxalin- 2(1H)-one | Intermediate 10 Amine No. 1 | ¹H NMR (400 MHz, CDCl₃): δ = 1.18 (d, 3H); 2.34-2.50 (m, 2H); 3.42 (s, 3H); 3.99-4.13 (m, 3H); 4.18 (d, 1H); 4.34 (d, 2H); 4.47 (d, 1H); 4.78-4.93 (m, 2H); 5.93 (s, 1H); 6.47 (s, 1H); 6.59 (d, 1H); 6.68 (d, 2H); 6.90 (d, 1H); 7.29-7.49 (m, 7H). |
| 17 | 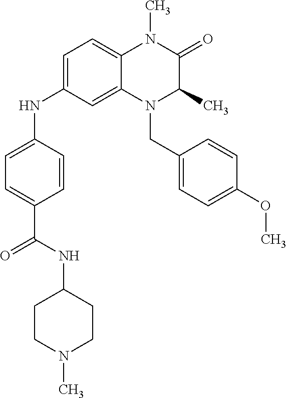 [SMILES: COc1ccc(CN2c3cc(Nc4ccc(C(=O)NC5CCN(C)CC5)cc4)ccc3N(C)C(=O)[C@H]2C)cc1] | 4-{[(3R)-4-(4- methoxybenzyl)-1,3- dimethyl-2-oxo-1,2,3,4- tetrahydroquinoxalin-6- yl]amino}-N-(1- methylpiperidin-4-yl) benzamide | Intermediate 16 Amine No. 2 | ¹H NMR (400 MHz, DMSO- d6): δ = 1.01 (d, 3H); 1.55 (qd, 2H); 1.69-1.75 (m, 2H); 1.92 (td, 2H); 2.15 (s, 3H); 2.75 (d, 2H); 3.28 (s, 3H); 3.64-3.73 (m, 1H); 3.77 (s, 3H); 3.96 (q, 1H); 4.20 (d, 1H); 4.37 (d, 1H); 6.47 (d, 1H); 6.57 (dd, 1H); 6.73 (d, 2H); 6.92 (d, 2H); 6.98 (d, 1H); 7.25 (d, 2H); 7.59 (d, 2H); 7.84 (d, 1H); 8.31 (s, 1H). |
| 18 | 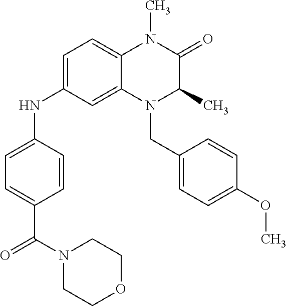 [SMILES: COc1ccc(CN2c3cc(Nc4ccc(C(=O)N5CCOCC5)cc4)ccc3N(C)C(=O)[C@H]2C)cc1] | (3R)-4-(4- methoxybenzyl)-1,3- dimethyl-6-{[4- (morpholin-4- ylcarbonyl)phenyl] amino}-3,4- dihydroquinoxalin- 2(1H)-one | Intermediate 16 Amine No. 3 | ¹H NMR (400 MHz, DMSO- d6): δ = 1.02 (d, 3H); 3.28 (s, 3H); 3.44-3.55 (m, 4H); 3.55- 3.65 (m, 4H); 3.75 (s, 3H); 3.98 (q, 1H); 4.22 (d, 1H); 4.38 (d, 1H); 6.44 (d, 1H); 6.56 (dd, 1H); 6.71 (d, 2H); 6.92 (d, 2H); 6.98 (d, 1H); 7.15 (d, 2H); 7.25 (d, 2H); 8.27 (s, 1H). |
| 19 | 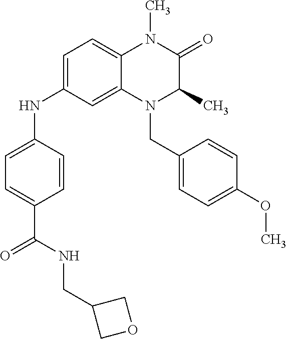 [SMILES: COc1ccc(CN2c3cc(Nc4ccc(C(=O)NCC5COC5)cc4)ccc3N(C)C(=O)[C@H]2C)cc1] | 4-{[(3R)-4-(4- methoxybenzyl)-1,3- dimethyl-2-oxo-1,2,3,4- tetrahydroquinoxalin-6- yl]amino}-N-(oxetan-3- ylmethyl)benzamide | Intermediate 16 Amine No. 4 | ¹H NMR (400 MHz, DMSO- d6): δ = 1.01 (d, 3H); 3.13 (sept, 1H); 3.28 (s, 3H); 3.50 (t, 2H); 3.76 (s, 3H); 3.96 (q, 1H); 4.20 (d, 1H); 4.33 (t, 2H); 4.38 (d, 1H); 4.62 (dd, 2H); 6.47 (d, 1H); 6.58 (dd, 1H); 6.75 (d, 2H); 6.92 (d, 2H); 6.98 (d, 1H); 7.25 (d, 2H); 7.59 (d, 2H); 8.25 (t, 1H); 8.33 (s, 1H). |
| 20 | 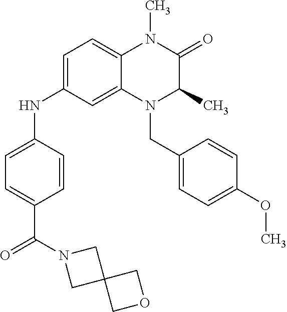 [SMILES: COc1ccc(CN2c3cc(Nc4ccc(C(=O)N5CC6(COC6)C5)cc4)ccc3N(C)C(=O)[C@H]2C)cc1] | (3R)-4-(4- methoxybenzyl)-1,3- dimethyl-6-{[4-(2-oxa- 6-azaspiro[3.3]hept-6- ylcarbonyl)phenyl] amino}-3,4- dihydroquinoxalin- 2(1H)-one | Intermediate 16 Amine No. 5 | ¹H NMR (400 MHz, DMSO- d6): δ = 1.02 (d, 3H); 3.29 (s, 3H); 3.77 (s, 3H); 4.00 (q, 1H); 4.08-4.29 (m, 2H); 4.24 (d, 1H); 4.35-4.56 (m, 2H); 4.38 (d, 1H); 4.69 (s, 4H); 6.44 (d, 1H); 6.56 (dd, 1H); 6.68 (d, 2H); 6.93 (d, 2H); 6.99 (d, 1H); 7.25 (d, 2H); 7.34 (d, 2H); 8.37 (s, 1H). |
| 21 | 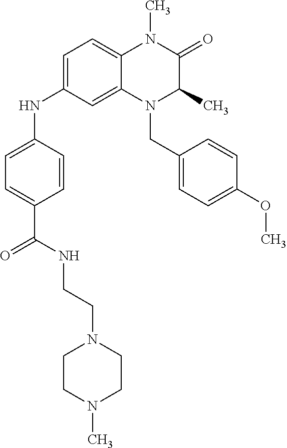 [SMILES: COc1ccc(CN2c3cc(Nc4ccc(C(=O)NCCN5CCN(C)CC5)cc4)ccc3N(C)C(=O)[C@H]2C)cc1] | 4-{[(3R)-4-(4- methoxybenzyl)-1,3- dimethyl-2-oxo-1,2,3,4- tetrahydroquinoxalin-6- yl]amino}-N-[2-(4- methylpiperazin-1- yl)ethyl]benzamide | Intermediate 16 Amine No. 6 | ¹H NMR (400 MHz, DMSO- d6): δ = 1.01 (d, 3H); 2.14 (s, 3H); 2.21-2.46 (m, 10H); 3.28 (s, 3H); 3.76 (s, 3H); 3.97 (q, 1H); 4.20 (d, 1H); 4.38 (d, 1H); 6.47 (d, 1H); 6.58 (dd, 1H); 6.75 (d, 2H); 6.92 (d, 2H); 6.98 (d, 1H); 7.25 (d, 2H); 7.57 (d, 2H); 7.98 (t, 1H); 8.31 (s, 1H). |
| 22 | 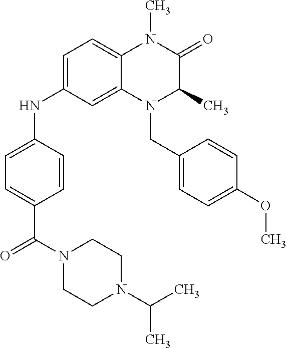 [SMILES: COc1ccc(CN2c3cc(Nc4ccc(C(=O)N5CCN(C(C)C)CC5)cc4)ccc3N(C)C(=O)[C@H]2C)cc1] | (3R)-4-(4- methoxybenzyl)-1,3- dimethyl-6-[(4-{[4- (propan-2-yl)piperazin- 1-yl]carbonyl} phenyl)amino]-3,4- dihydroquinoxalin- 2(1H)-one | Intermediate 16 Amine No. 7 | ¹H NMR (400 MHz, DMSO- d6): δ = 0.98 (d, 6H); 1.02 (d, 3H); 2.40-2.47 (m, 4H); 2.68 (sept, 1H); 3.28 (s, 3H); 3.42- 3.52 (m, 4H); 3.75 (s, 3H); 3.99 (q, 1H); 4.22 (d, 1H); 4.39 (d, 1H); 6.44 (d, 1H); 6.55 (dd, 1H); 6.70 (d, 2H); 6.92 (d, 2H); 6.97 (d, 1H); 7.11 (d, 2H); 7.25 (d, 2H); 8.24 (s, 1H). |
| 23 | 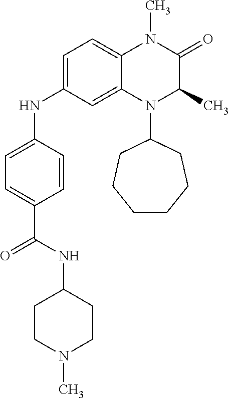 [SMILES: C[C@@H]1C(=O)N(C)c2ccc(Nc3ccc(C(=O)NC4CCN(C)CC4)cc3)cc2N1C1CCCCCC1] | 4-{[(3R)-4-cycloheptyl- 1,3-dimethyl-2-oxo- 1,2,3,4- tetrahydroquinoxalin-6- yl]amino}-N-(1- methylpiperidin-4- yl)benzamide | Intermediate 20 Amine No. 2 | ¹H NMR (400 MHz, CDCl₃): δ = 1.13 (d, 3H); 1.37-1.89 (m, 13H); 1.99-2.10 (m, 3H); 2.17 (t, 2H); 2.31 (s, 3H); 2.78-2.88 (m, 2H); 3.37 (s, 3H); 3.48 (tt, 1H); 3.93-4.07 (m, 1H); 4.20 (q, 1H); 5.83 (d, 1H); 5.88 (s, 1H); 6.58 (d, 1H); 6.65 (dd, 1H); 6.89 (d, 1H); 6.98 (d, 2H); 7.66 (d, 2H). |
| 24 | 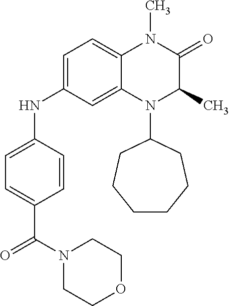 [SMILES: C[C@@H]1C(=O)N(C)c2ccc(Nc3ccc(C(=O)N4CCOCC4)cc3)cc2N1C1CCCCCC1] | (3R)-4-cycloheptyl-1,3- dimethyl-6-{[4- (morpholin-4- ylcarbonyl)phenyl] amino}-3,4- dihydroquinoxalin- 2(1H)-one | Intermediate 20 Amine No. 3 | ¹H NMR (400 MHz, CDCl₃): δ = 1.13 (d, 3H); 1.37-1.90 (m, 12H); 2.00-2.09 (m, 1H); 3.37 (s, 3H); 3.47 (tt, 1H); 3.61-3.78 (m, 8H); 4.20 (q, 1H); 5.83 (s, 1H); 6.57 (d, 1H); 6.64 (dd, 1H); 6.88 (d, 1H); 6.99 (d, 2H); 7.35 (d, 2H). |
| 25 | 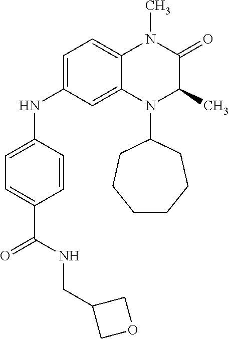 [SMILES: C[C@@H]1C(=O)N(C)c2ccc(Nc3ccc(C(=O)NCC4COC4)cc3)cc2N1C1CCCCCC1] | 4-{[(3R)-4-cycloheptyl- 1,3-dimethyl-2-oxo- 1,2,3,4- tetrahydroquinoxalin-6- yl]amino}-N-(oxetan-3- ylmethyl)benzamide | Intermediate 20 Amine No. 4 | ¹H NMR (400 MHz, CDCl₃): δ = 1.13 (d, 3H); 1.37-1.90 (m, 14H); 2.00-2.09 (m, 1H); 3.25- 3.36 (m, 1H); 3.37 (s, 3H); 3.47 (tt, 1H); 3.75 (t, 2H); 4.20 (q, 1H); 4.49 (t, 2H); 4.84 (dd, 2H); 5.91 (s, 1H); 6.26 (t, 1H); 6.58 (d, 1H); 6.66 (dd, 1H); 6.89 (d, 1H); 6.98 (d, 2H); 7.67 (d, 2H). |
| 26 | 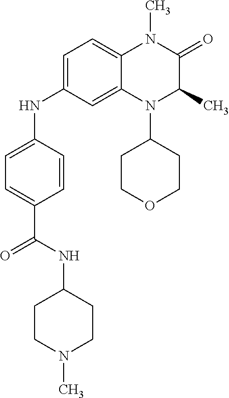 [SMILES: C[C@@H]1C(=O)N(C)c2ccc(Nc3ccc(C(=O)NC4CCN(C)CC4)cc3)cc2N1C1CCOCC1] | 4-{[(3R)-1,3-dimethyl- 2-oxo-4-(tetrahydro- 2H-pyran-4-yl)-1,2,3,4- tetrahydroquinoxalin-6- yl]amino}-N-(1- methylpiperidin-4- yl)benzamide | Intermediate 24 Amine No. 2 | ¹H NMR (400 MHz, CDCl₃): δ = 1.13 (d, 3H); 1.57 (qd, 2H); 1.66-1.75 (m, 3H); 1.78-1.96 (m, 3H); 2.01-2.10 (m, 2H); 2.17 (t, 2H); 2.31 (s, 3H); 2.78- 2.88 (m, 2H); 3.38 (s, 3H); 3.39-3.50 (m, 2H); 3.55 (tt, 1H); 3.93-4.10 (m, 3H); 4.14 (q, 1H); 5.84 (d, 1H); 5.88 (s, 1H); 6.67 (d, 1H); 6.74 (dd, 1H); 6.91 (d, 1H); 6.97 (d, 2H); 7.67 (d, 2H). |
| 27 | 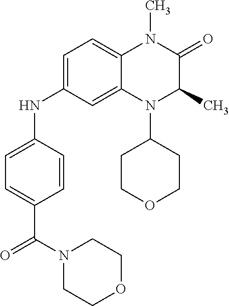 [SMILES: C[C@@H]1C(=O)N(C)c2ccc(Nc3ccc(C(=O)N4CCOCC4)cc3)cc2N1C1CCOCC1] | (3R)-1,3-dimethyl-6- {[4-(morpholin-4- ylcarbonyl)phenyl] amino}-4-(tetrahydro- 2H-pyran-4-yl)-3,4- dihydroquinoxalin- 2(1H)-one | Intermediate 24 Amine No. 3 | ¹H NMR (400 MHz, CDCl₃): δ = 1.13 (d, 3H); 1.70 (qd, 1H); 1.75-1.95 (m, 3H); 3.37 (s, 3H); 3.44 (qd, 2H); 3.55 (tt, 1H); 3.62-3.77 (m, 8H); 4.05 (td, 2H); 4.14 (q, 1H); 5.80 (s, 1H); 6.66 (d, 1H); 6.74 (dd, 1H); 6.91 (d, 1H); 6.97 (d, 2H); 7.35 (d, 2H). |
| 28 | 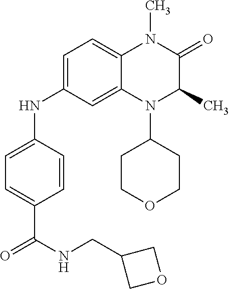 [SMILES: C[C@@H]1C(=O)N(C)c2ccc(Nc3ccc(C(=O)NCC4COC4)cc3)cc2N1C1CCOCC1] | 4-{[(3R)-1,3-dimethyl- 2-oxo-4-(tetrahydro- 2H-pyran-4-yl)-1,2,3,4- tetrahydroquinoxalin-6- yl]amino}-N-(oxetan-3- ylmethyl)benzamide | Intermediate 24 Amine No. 4 | ¹H NMR (400 MHz, CDCl₃): δ = 1.13 (d, 3H); 1.70 (dq, 1H); 1.76-1.95 (m, 3H); 3.30 (sept, 1H); 3.38 (s, 3H); 3.39-3.50 (m, 2H); 3.55 (tt, 1H); 3.75 (t, 2H); 4.05 (td, 2H); 4.14 (q, 1H); 4.49 (t, 2H); 4.85 (dd, 2H); 5.90 (s, 1H); 6.26 (t, 1H); 6.67 (d, 1H); 6.74 (dd, 1H); 6.92 (d, 1H); 6.96 (d, 2H); 7.68 (d, 2H). |
| 29 | 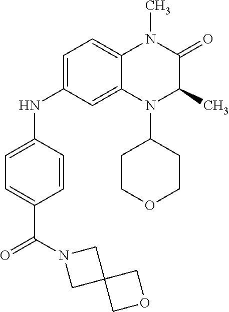 [SMILES: C[C@@H]1C(=O)N(C)c2ccc(Nc3ccc(C(=O)N4CC5(COC5)C4)cc3)cc2N1C1CCOCC1] | (3R)-1,3-dimethyl-6- {[4-(2-oxo-6- azaspiro[3.3]hept-6- ylcarbonyl)phenyl] amino}-4-(tetrahydro- 2H-pyran-4-yl)-3,4- dihydroquinoxalin- 2(1H)-one | Intermediate 24 Amine No. 5 | ¹H NMR (400 MHz, CDCl₃): δ = 1.13 (d, 3H); 1.71 (dq, 1H); 1.77-1.97 (m, 3H); 3.38 (s, 3H); 3.40-3.51 (m, 2H); 3.56 (tt, 1H); 4.06 (td, 2H); 4.15 (q, 1H); 4.29-4.55 (m, 4H); 4.82 (s, 4H); 5.88 (s, 1H); 6.66 (d, 1H); 6.75 (dd, 1H); 6.92 (d, 1H); 6.94 (d, 2H); 7.56 (d, 2H). |
| 30 | 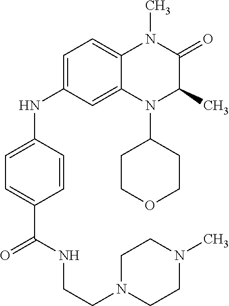 [SMILES: C[C@@H]1C(=O)N(C)c2ccc(Nc3ccc(C(=O)NCCN4CCN(C)CC4)cc3)cc2N1C1CCOCC1] | 4-{[(3R)-1,3-dimethyl- 2-oxo-4-(tetrahydro- 2H-pyran-4-yl)-1,2,3,4- tetrahydroquinoxalin-6- yl]amino}-N-[2-(4- methylpiperazin-1- yl)ethyl]benzamide | Intermediate 24 Amine No. 6 | ¹H NMR (400 MHz, CDCl₃): δ = 1.13 (d, 3H); 1.74-1.97 (m, 4H); 2.31 (s, 3H); 2.36-2.67 (8H); 2.61 (t, 2H); 3.38 (s, 3H); 3.40-3.63 (m, 5H); 3.99- 4.11 (m, 2H); 4.15 (q, 1H); 5.89 (s, 1H); 6.56-6.72 (m, 2H); 6.75 (dd, 1H); 6.92 (d, 1H); 6.98 (d, 2H); 7.69 (d, 2H). |
| 31 | 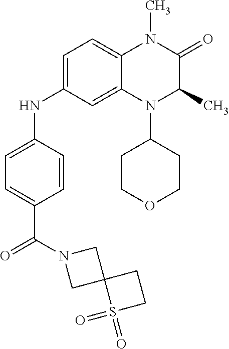 [SMILES: C[C@@H]1C(=O)N(C)c2ccc(Nc3ccc(C(=O)N4CC5(CCS5(=O)=O)C4)cc3)cc2N1C1CCOCC1] | (3R)-6-({4-[(1,1- dioxido-1-thia-6- azaspiro[3.3]hept-6- yl)carbonyl]phenyl} amino)-1,3-dimethyl- 4-(tetrahydro-2H- pyran-4-yl)-3,4- dihydroquinoxalin- 2(1H)-one | Intermediate 24 Amine No. 1 | ¹H NMR (400 MHz, CDCl₃): δ = 1.13 (d, 3H); 1.71 (dq, 1H); 1.77-1.97 (m, 3H); 2.42 (t, 2H); 3.38 (s, 3H); 3.40-3.63 (m, 3H); 3.98-4.12 (m, 4H); 4.15 (q, 1H); 4.37 (d, 2H); 4.88 (d, 2H); 5.92 (s, 1H); 6.67 (d, 1H); 6.75 (dd, 1H); 6.93 (d, 1H); 6.94 (d, 2H); 7.56 (d, 2H). |
| 32 | 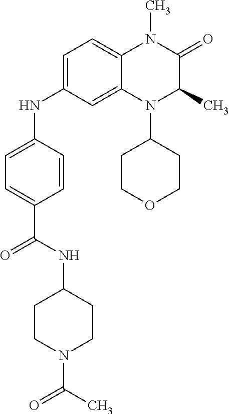 [SMILES: CC(=O)N1CCC(NC(=O)c2ccc(Nc3ccc4c(c3)N(C3CCOCC3)[C@H](C)C(=O)N4C)cc2)CC1] | N-(1-acetylpiperidin-4- yl)-4-{[(3R)-1,3- dimethyl-2-oxo-4- (tetrahydro-2H-pyran-4- yl)-1,2,3,4- tetrahydroquinoxalin-6- yl]amino}benzamide | Intermediate 24 Amine No. 8 | ¹H NMR (400 MHz, CDCl₃): δ = 1.13 (d, 3H); 1.41 (q, 2H); 1.70 (d, 1H); 1.76-1.96 (m, 3H); 2.05 (d, 1H); 2.12 (s, 3H); 2.18 (d, 1H); 2.78 (t, 1H); 3.23 (t, 1H); 3.38 (s, 3H); 3.50-3.63 (m, 3H); 3.84 (d, 1H); 4.05 (t, 2H); 4.15 (q, 1H); 4.17-4.29 (m, 1H); 4.62 (d, 1H); 5.88 (d, 1H); 5.90 (s, 1H); 6.67 (d, 1H); 6.74 (dd, 1H); 6.92 (d, 1H); 6.97 (d, 2H); 7.67 (d, 2H). |
| 33 | 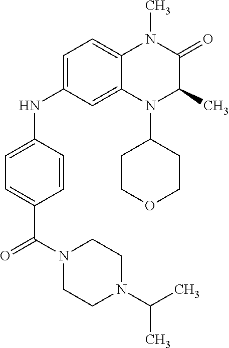 [SMILES: CC(C)N1CCN(C(=O)c2ccc(Nc3ccc4c(c3)N(C3CCOCC3)[C@H](C)C(=O)N4C)cc2)CC1] | (3R)-1,3-dimethyl-6- [(4-{[4-(propan-2- yl)piperazin-1- yl]carbonyl}phenyl) amino]-4-(tetrahydro- 2H-pyran-yl)-3,4- dihydroquinoxalin- 2(1H)-one | Intermediate 24 Amine No. 7 | ¹H NMR (400 MHz, CDCl₃): δ = 1.06 (d, 6H); 1.13 (d, 3H); 1.70 (d, 1H); 1.76-1.96 (m, 3H); 2.45-2.63 (m, 4H); 2.73 (sept, 1H); 3.37 (s, 3H); 3.39- 3.81 (m, 7H); 4.05 (t, 2H); 4.14 (q, 1H); 5.79 (s, 1H); 6.65 (d, 1H); 6.73 (dd, 1H); 6.90 (d, 1H); 6.97 (d, 2H); 7.35 (d, 2H). |
| 34 | 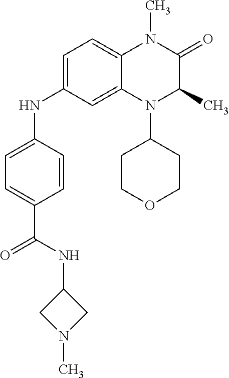 [SMILES: C[C@@H]1C(=O)N(C)c2ccc(Nc3ccc(C(=O)NC4CN(C)C4)cc3)cc2N1C1CCOCC1] | 4-{[(3R)-1,3-dimethyl- 2-oxo-4-(tetrahydro- 2H-pyran-4-yl)-1,2,3,4- tetrahydroquinoxalin-6- yl]amino}-N-(1- methylazetidin-3- yl)benzamide | Intermediate 24 Amino No. 9 | ¹H NMR (400 MHz, CDCl₃): δ = 1.13 (d, 3H); 1.65-1.97 (m, 6H); 2.38 (s, 3H); 3.12 (t, 2H); 3.38 (s, 3H); 3.40-3.62 (m, 3H); 3.68 (t, 2H); 4.06 (t, 2H); 4.15 (q, 1H); 4.64-4.78 (m, 1H); 5.90 (s, 1H); 6.56 (d, 1H); 6.67 (d, 1H); 6.74 (dd, 1H); 6.93 (d, 1H); 6.96 (d, 2H); 7.70 (d, 2H). |
| 35 | 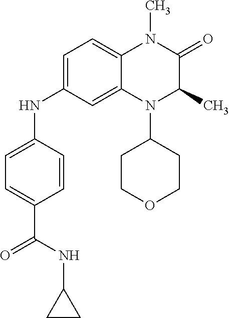 [SMILES: C[C@@H]1C(=O)N(C)c2ccc(Nc3ccc(C(=O)NC4CC4)cc3)cc2N1C1CCOCC1] | N-cyclopropyl-4- {[(3R)-1,3-dimethyl-2- oxo-4-(tetrahydro-2H- pyran-4-yl)-1,2,3,4- tetrahydroquinoxalin-6- yl]amino}benzamide | Intermediate 24 Amine No. 10 | ¹H NMR (400 MHz, CDCl₃): δ = 0.56-0.66 (m, 2H); 0.87 (q, 2H); 1.13 (d, 3H); 1.70 (d, 1H); 1.76-1.97 (m, 3H); 2.84- 2.95 (m, 1H); 3.38 (s, 3H); 3.39-3.63 (m, 3H); 4.05 (t, 2H); 4.14 (q, 1H); 5.89 (s, 1H); 6.13 (s, 1H); 6.67 (d, 1H); 6.73 (dd, 1H); 6.92 (d, 1H); 6.95 (d, 2H); 7.65 (d, 2H). |
| 36 | 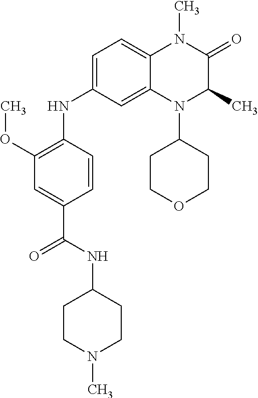 [SMILES: COc1cc(C(=O)NC2CCN(C)CC2)ccc1Nc1ccc2c(c1)N(C1CCOCC1)[C@H](C)C(=O)N2C] | 4-{[(3R)-1,3-dimethyl- 2-oxo-4-(tetrahydro- 2H-pyran-4-yl)-1,2,3,4- tetrahydroquinoxalin-6- yl]amino}-3-methoxy- N-(1-methylpiperidin-4- yl)benzamide | Intermediate 26 Amine No. 2 | ¹H NMR (400 MHz, DMSO- d6): δ = 0.98 (d, 3H); 1.51- 1.83 (m, 7H); 1.83-1.93 (m, 1H); 2.03 (t, 2H); 2.21 (s, 3H); 2.77-2.89 (m, 2H); 3.25 (s, 3H); 3.90 (s, 3H); 4.07 (q, 1H); 6.75 (dd, 1H); 6.83 (d, 1H); 6.98 (d, 1H); 7.13 (d, 1H); 7.40 (d, 1H); 7.43 (s, 1H); 7.53 (s, 1H); 7.98 (d, 1H). |
| 37 | 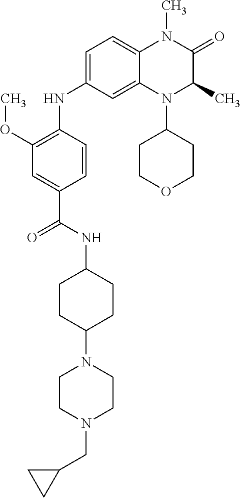 [SMILES: COc1cc(C(=O)NC2CCC(N3CCN(CC4CC4)CC3)CC2)ccc1Nc1ccc2c(c1)N(C1CCOCC1)[C@H](C)C(=O)N2C] | N-{4-[4- (cyclopropylmethyl) piperazin-1-yl] cyclohexyl}-4-{[(3R)- 1,3-dimethyl-2-oxo-4- (tetrahydro-2H-pyran-4- yl)-1,2,3,4- tetrahydroquinoxalin-6- yl]amino}-3- methoxybenzamide | Intermediate 26 Amine No. 11 | ¹H NMR (400 MHz, DMSO- d6): δ = 0.01-0.09 (m, 2H); 0.39-0.49 (m, 2H); 0.72-0.87 (m, 1H); 0.98 (d, 3H); 1.22- 1.44 (m, 4H); 1.60 (bd, 1H); 1.64-1.95 (m, 8H); 2.14 (d, 2H); 2.15-2.29 (m, 1H); 2.43 (bs, 4H); 3.25 (s, 3H); 3.85- 3.99 (m + s, 5H); 4.06 (q, 1H); 6.75 (dd, 1H); 6.83 (d, 1H); 6.98 (d, 1H); 7.13 (d, 1H); 7.38 (dd, 1H); 7.43 (d, 1H); 7.49 (s, 1H); 7.90 (d, 1H). |
| 38 | 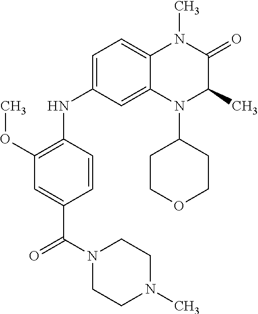 [SMILES: COc1cc(C(=O)N2CCN(C)CC2)ccc1Nc1ccc2c(c1)N(C1CCOCC1)[C@H](C)C(=O)N2C] | (3R)-6-({2-methoxy-4- [(4-methylpiperazin-1- yl)carbonyl]phenyl} amino)-1,3-dimethyl-4- (tetrahydro-2H-pyran-4- yl)-3,4- dihydroquinoxalin- 2(1H)-one | Intermediate 26 Amino No. 12 | ¹H NMR (400 MHz, DMSO- d6): δ = 0.98 (d, 3H); 1.59 (bd, 1H); 1.64-1.83 (m, 2H); 1.88 (bd, 1H); 2.20 (s, 3H); 2.26- 2.37 (m, 4H); 3.24 (s, 3H); 3.33-3.46 (m, 2H); 3.46-3.66 (m, 5H); 3.83-3.99 (m + s, 5H); 4.06 (q, 1H); 6.73 (dd, 1H); 6.81 (d, 1H); 6.88 (dd, 1H); 6.96 (d, 1H); 6.98 (s, 1H); 7.13 (d, 1H); 7.43 (s, 1H). |
| 39 | 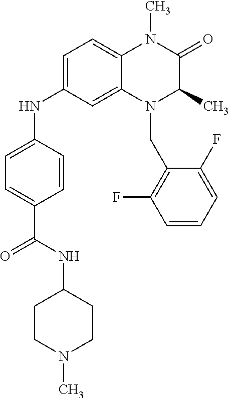 [SMILES: C[C@@H]1C(=O)N(C)c2ccc(Nc3ccc(C(=O)NC4CCN(C)CC4)cc3)cc2N1Cc1c(F)cccc1F] | 4-{[4-(2,6- difluorobenzyl)-1,3- dimethyl-2-oxo-1,2,3,4- tetrahydroquinoxalin-6- yl]amino}-N-(1- methylpiperidin-4- yl)benzamide | Intermediate 32 Amine No. 2 | ¹H NMR (400 MHz, DMSO- d6): δ = 1.03 (d, 3H); 1.47- 1.63 (m, 2H); 1.72 (d, 2H); 1.85-1.97 (m, 2H); 2.14 (s, 3H); 2.77 (s, 1H); 2.73 (s, 1H); 3.22 (s, 3H); 3.61-3.76 (m, 1H); 3.82 (q, 1H); 4.25 (d, 1H); 4.57 (d, 1H); 6.64 (dd, 1H); 6.73 (d, 1H); 6.88-7.02 (m, 3H); 7.08-7.20 (m, 2H); 7.39-7.52 (m, 1H); 7.70 (d, 2H); 7.89 (d, 1H); 8.38 (s, 1H). |
| 40 | 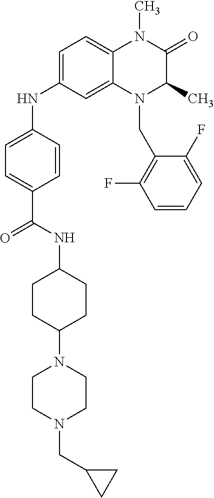 [SMILES: C[C@@H]1C(=O)N(C)c2ccc(Nc3ccc(C(=O)NC4CCC(N5CCN(CC6CC6)CC5)CC4)cc3)cc2N1Cc1c(F)cccc1F] | N-{4-[4- (cyclopropylmethyl) piperazin-1-yl] cyclohexyl}-4-{[4- (2,6-difluorobenzyl)- 1,3-dimethyl-2-oxo- 1,2,3,4- tetrahydroquinoxalin-6- yl]amino}benzamide | Intermediate 32 Amine No. 11 | ¹H NMR (400 MHz, DMSO- d6): δ = 0.03 (q, 2H); 0.38- 0.47 (m, 2H); 0.73-0.86 (m, 1H); 1.03 (d, 3H); 1.19-1.42 (m, 5H); 1.75-1.91 (m, 4H); 2.12 (d, 2H); 2.40 (bs, 3H); 3.31 (s, 8H); 3.69 (bs, 1H); 3.81 (q, 1H); 4.25 (d, 1H); 4.56 (d, 1H); 6.63 (dd, 1H); 6.73 (d, 1H); 6.99 (d, 1H); 6.92 (d, 2H); 7.08-7.19 (m, 2H); 7.39-7.52 (m, 1H); 7.69 (d, 2H); 7.86 (d, 1H); 8.38 (s, 1H). |

Tables 2a and 2b

In analogy to the preparation of Example 3, the examples shown in Table 2b were prepared from the intermediates thereof specified in each case and from the sulphonamides shown in Table 2a:

TABLE 2a

| Sulphon- | | |
|---|---|---|
| amide | | CAS |
| No. | Structure | number |
| 1 | 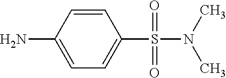 | 1709-59-7 |
| 2 | 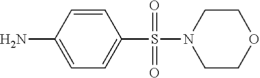 [SMILES: Nc1ccc(S(=O)(=O)N2CCOCC2)cc1] | 21626-70-0 |
| 3 | 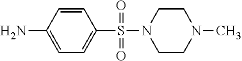 [SMILES: CN1CCN(S(=O)(=O)c2ccc(N)cc2)CC1] | 21623-68-7 |
| 4 | 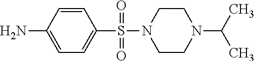 [SMILES: CC(C)N1CCN(S(=O)(=O)c2ccc(N)cc2)CC1] | 524719-43-5 |

TABLE 2b

| Ex. | Structure | Name | Intermediate | Anaylsis |
|---|---|---|---|---|
| 41 | 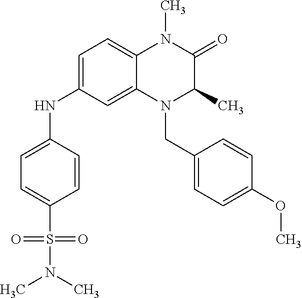 [SMILES: COc1ccc(CN2c3cc(Nc4ccc(S(=O)(=O)N(C)C)cc4)ccc3N(C)C(=O)[C@H]2C)cc1] | 4-{[(3R)-4-(4- methoxybenzyl)-1,3- dimethyl-2-oxo-1,2,3,4- tetrahydroquinoxalin-6- yl]amino}-N,N- dimethylbenzene- sulphonamide | Intermediate 14, sulphonamide No. 1 | ¹H NMR (400 MHz, DMSO- d6): δ = 1.04 (d, 3H); 2.54 (s, 6H); 3.30 (s, 3H); 3.77 (s, 3H); 4.04 (q, 1H); 4.26 (d, 1H); 4.40 (d, 1H); 6.43 (d, 1H); 6.58 (dd, 1H); 6.71 (d, 2H); 6.91 (d, 2H); 7.02 (d, 1H); 7.25 (d, 2H); 7.32 (d, 2H); 8.66 (s, 1H). |
| 42 | 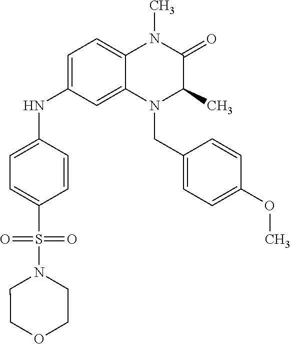 [SMILES: COc1ccc(CN2c3cc(Nc4ccc(S(=O)(=O)N5CCOCC5)cc4)ccc3N(C)C(=O)[C@H]2C)cc1] | (3R)-4-(4- methoxybenzyl)-1,3- dimethyl-6-{[4- (morpholin-4- ylsulphonyl)phenyl] amino}-3,4- dihydroquinoxalin- 2(1H)-one | Intermediate 14, sulphonamide No. 2 | ¹H NMR (400 MHz, DMSO- d6): δ = 1.04 (d, 3H); 2.74- 2.83 (m, 4H); 3.30 (s, 3H); 3.59-3.68 (m, 4H); 3.76 (s, 3H); 4.05 (q, 1H); 4.26 (d, 1H); 4.40 (d, 1H); 6.44 (d, 1H); 6.58 (dd, 1H); 6.72 (d, 2H); 6.92 (d, 2H); 7.02 (d, 1H); 7.25 (d, 2H); 7.31 (d, 2H); 8.71 (s, 1H). |
| 43 | 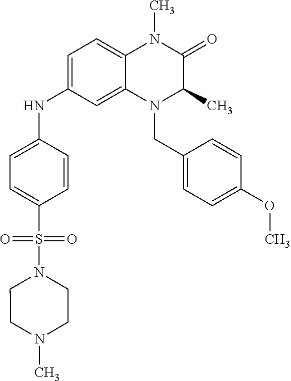 [SMILES: COc1ccc(CN2c3cc(Nc4ccc(S(=O)(=O)N5CCN(C)CC5)cc4)ccc3N(C)C(=O)[C@H]2C)cc1] | (3R)-4-(4- methoxybenzyl)-1,3- dimethyl-6-({4-[(4- methylpiperazin-1- yl)sulphonyl]phenyl} amino)-3,4- dihydroquinoxalin- 2(1H)-one | Intermediate 14, sulphonamide No. 3 | ¹H NMR (400 MHz, DMSO- d6): δ = 1.04 (d, 3H); 2.15 (s, 3H); 2.32-2.39 (m, 4H); 2.76- 2.87 (m, 4H); 3.30 (s, 3H); 3.76 (s, 3H); 4.04 (q, 1H); 4.26 (d, 1H); 4.40 (d, 1H); 6.44 (d, 1H); 6.58 (dd, 1H); 6.73 (d, 2H); 6.91 (d, 2H); 7.02 (d, 1H); 7.25 (d, 2H); 7.31 (d, 2H); 8.66 (s, 1H). |
| 44 | 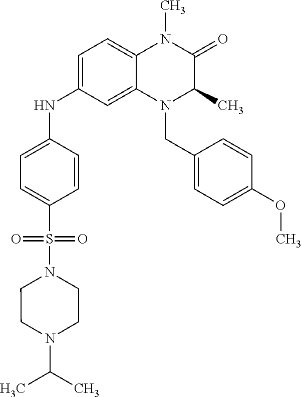 [SMILES: COc1ccc(CN2c3cc(Nc4ccc(S(=O)(=O)N5CCN(C(C)C)CC5)cc4)ccc3N(C)C(=O)[C@H]2C)cc1] | (3R)-4-(4- methoxybenzyl)-1,3- dimethyl-6-[(4-{[4- (propan-2-yl)piperazin- 1-yl]sulphonyl} phenyl)amino]-3,4- dihydroquinoxalin- 2(1H)-one | Intermediate 14, sulphonamide No. 4 | ¹H NMR (400 MHz, CDCl₃): δ = 1.02 (d, 6H); 1.17 (d, 3H); 2.55-2.74 (m, 5H); 2.93-3.11 (m, 4H); 3.42 (s, 3H); 3.84 (s, 3H); 4.06 (q, 1H); 4.13 (d, 1H); 4.42 (d, 1H); 5.96 (s, 1H); 6.48 (d, 1H); 6.60 (dd, 1H); 6.70 (d, 2H); 6.88 (d, 2H); 6.91 (d, 1H); 7.24 (d, 2H); 7.44 (d, 2H). |
| 45 | 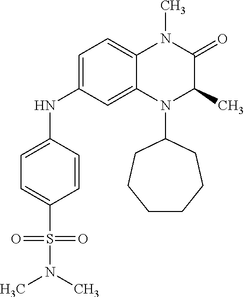 [SMILES: C[C@@H]1C(=O)N(C)c2ccc(Nc3ccc(S(=O)(=O)N(C)C)cc3)cc2N1C1CCCCCC1] | 4-{[(3R)-4-cycloheptyl- 1,3-dimethyl-2-oxo- 1,2,3,4- tetrahydroquinoxalin-6- yl]amino}-N,N- dimethylbenzene- sulphonamide | Intermediate 18, sulphonamide No. 1 | ¹H NMR (400 MHz, CDCl₃): δ = 1.13 (d, 3H); 1.35-1.90 (m, 11H); 1.98-2.10 (m, 1H); 2.69 (s, 6H); 3.38 (s, 3H); 3.47 (tt, 1H); 4.22 (q, 1H); 6.05 (s, 1H); 6.59 (d, 1H); 6.69 (dd, 1H); 6.91 (d, 1H); 7.01 (d, 2H); 7.62 (d, 2H). |
| 46 | 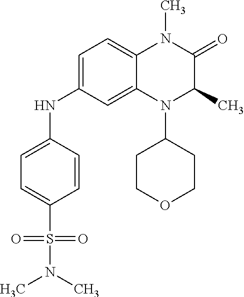 [SMILES: C[C@@H]1C(=O)N(C)c2ccc(Nc3ccc(S(=O)(=O)N(C)C)cc3)cc2N1C1CCOCC1] | 4-{[(3R)-1,3-dimethyl- 2-oxo-4-(tetrahydro- 2H-pyran-4-yl)-1,2,3,4- tetrahydroquinoxalin-6- yl]amino}-N,N- dimethylbenzene- sulphonamide | Intermediate 22, sulphonamide No. 1 | ¹H NMR (400 MHz, CDCl₃): δ = 1.14 (d, 3H); 1.71 (d, 1H); 1.77-1.98 (m, 3H); 2.70 (s, 6H); 3.39 (s, 3H); 3.41-3.64 (m, 3H); 4.06 (t, 2H); 4.16 (q, 1H); 6.02 (s, 1H); 6.69 (s, 1H); 6.79 (d, 1H); 6.95 (d, 1H); 6.98 (d, 2H); 7.62 (d, 2H). |
| 47 | 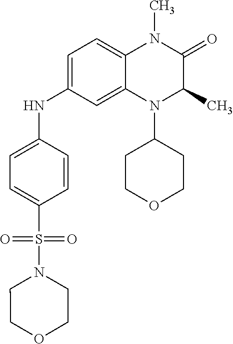 [SMILES: C[C@@H]1C(=O)N(C)c2ccc(Nc3ccc(S(=O)(=O)N4CCOCC4)cc3)cc2N1C1CCOCC1] | (3R)-1,3-dimethyl-6- {[4-(morpholin-4- ylsulphonyl)phenyl] amino}-4-(tetrahydro- 2H-pyran-4-yl)-3,4- dihydroquinoxalin- 2(1H)-one | Intermediate 22, sulphonamide No. 2 | ¹H NMR (400 MHz, CDCl₃): δ = 1.14 (d, 3H); 1.71 (d, 1H); 1.77-1.99 (m, 3H); 2.94-3.08 (m, 4H); 3.39 (s, 3H); 3.42- 3.65 (m, 3H); 3.70-3.83 (m, 4H); 4.07 (t, 2H); 4.17 (q, 1H); 6.03 (s, 1H); 6.69 (s, 1H); 6.80 (d, 1H); 6.91-7.04 (m, 3H); 7.59 (d, 2H). |
| 48 | 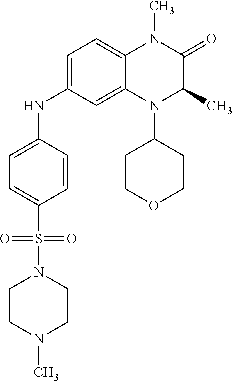 [SMILES: C[C@@H]1C(=O)N(C)c2ccc(Nc3ccc(S(=O)(=O)N4CCN(C)CC4)cc3)cc2N1C1CCOCC1] | (3R)-1,3-dimethyl-6- ({4-[(4-methylpiperazin- 1-yl)sulphonyl]phenyl} amino)-4-(tetrahydro- 2H-pyran-4-yl)-3,4- dihydroquinoxalin- 2(1H)-one | Intermediate 22, sulphonamide No. 3 | ¹H NMR (400 MHz, CDCl₃): δ = 1.14 (d, 3H); 1.71 (d, 1H); 1.77-1.99 (m, 3H); 2.30 (s, 3H); 2.42-2.61 (m, 4H); 2.94- 3.15 (m, 4H); 3.39 (s, 3H); 3.42-3.66 (m, 3H); 4.06 (t, 2H); 4.16 (q, 1H); 5.98 (s, 1H); 6.66 (s, 1H); 6.76 (d, 1H); 6.90-6.99 (m, 3H); 7.58 (d, 2H). |

Biological Efficacy of the Inventive Compounds

Protein-Protein Interaction Assay: BRD4/Acetylated Peptide H4 Binding Assay

1. Assay Description for BRD4 Bromo Domain 1 [BRD4(1)]

To assess the BRD4(1) binding strength of the substances described in this application, the ability thereof to inhibit the interaction between BRD4(1) and acetylated histone H4 in a dose-dependent manner was quantified.

For this purpose, a time-resolved fluorescence resonance energy transfer (TR-FRET) assay was used, which measures the binding between N-terminally His6-tagged BRD4(1) (amino acids 67-152) and a synthetic acetylated histone H4 (Ac-H4) peptide with sequence GRGK(Ac)GGK(Ac)GLGK(Ac)GGAK(Ac)RHGSGSK-biotin. The recombinant BRD4(1) protein produced in house according to Filippakopoulos et al., Cell, 2012, 149:214-231 was expressed in E. coli and purified by means of (Ni-NTA) affinity and (Sephadex G-75) size exclusion chromatography. The Ac-H4 peptide can be purchased, for example, from Biosyntan (Berlin, Germany).

In the assay, typically 11 different concentrations of each substance (0.1 nM, 0.33 nM, 1.1 nM, 3.8 nM, 13 nM, 44 nM, 0.15 μM, 0.51 μM, 1.7 μM, 5.9 μM and 20 μM) were analysed as duplicates on the same microtitre plate. For this purpose, 100-fold concentrated solutions in DMSO were prepared by serial dilutions (1:3.4) of a 2 mM stock solution into a clear, 384-well microtitre plate (Greiner Bio-One, Frickenhausen, Germany). From this, 50 nl were transferred into a black test plate (Greiner Bio-One, Frickenhausen, Germany). The test was started by the addition of 2 μl of a 2.5-fold concentrated BRD4(1) solution (final concentration typically 10 nM in the 5 μl of reaction volume) in aqueous assay buffer [50 mM HEPES pH 7.5, 50 mM sodium chloride (NaCl), 0.25 mM CHAPS and 0.05% serum albumin (BSA)] to the substances in the test plate. This was followed by a 10-minute incubation step at 22° C. for the pre-equilibration of putative complexes between BRD4(1) and the substances. Subsequently, 3 μl of a 1.67-fold concentrated solution (in assay buffer) consisting of Ac-H4 peptide (83.5 nM) and TR-FRET detection reagents [16.7 nM anti-6His-XL665 and 3.34 nM streptavidin cryptate (both from Cisbio Bioassays, Codolet, France), and 668 mM potassium fluoride (KF)] were added.

The mixture was then incubated in the dark at 22° C. for one hour and then at 4° C. for at least 3 hours and for no longer than overnight. The formation of BRD4(1)/Ac-H4 complexes was determined by the measurement of the resonance energy transfer from the streptavidin-Eu cryptate to the anti-6His-XL665 antibody present in the reaction. For this purpose, the fluorescence emission was measured at 620 nm and 665 nm after excitation at 330-350 nm in a TR-FRET measuring instrument, for example a Rubystar or Pherastar (both from BMG Lab Technologies, Offenburg, Germany) or a Viewlux (Perkin-Elmer). The ratio of the emissions at 665 nm and at 622 nm was taken as an indicator of the amount of BRD4(1)/Ac-H4 complexes formed.

The data (ratios) obtained were normalized, with 0% inhibition corresponding to the mean from the measurements for a set of controls (typically 32 data points) in which all the reagents were present. In these, in place of test substances, 50 nl of DMSO (100%) were used Inhibition of 100% corresponded to the mean from the measurements for a set of controls (typically 32 data points) in which all the reagents except BRD4(1) were present. The IC₅₀ was determined by regression analysis based on a 4-parameter equation (minimum, maximum, IC₅₀, Hill; Y=max+(min−max)/(1+(X/IC₅₀)Hill).

2. Assay Description for BRD4 Bromo Domain 2 [BRD4(2)]

To assess the BRD4(2) binding strength of the substances described in this application, the ability thereof to inhibit the interaction between BRD4(2) and acetylated histone H4 in a dose-dependent manner was quantified.

For this purpose, a time-resolved fluorescence resonance energy transfer (TR-FRET) assay was used, which measures the binding between N-terminally His6-tagged BRD4(2) (amino acids 357-445) and a synthetic acetylated histone H4 (Ac-H4) peptide with sequence SGRGK(Ac)GGK(Ac)GLGK(Ac)GGAK(Ac)RHRKVLRDNGSGSK-biotin. The recombinant BRD4(2) protein produced in house according to Filippakopoulos et al., Cell, 2012, 149:214-231 was expressed in E. coli and purified by means of (Ni-NTA) affinity and (Sephadex G-75) size exclusion chromatography. The Ac-H4 peptide can be purchased, for example, from Biosyntan (Berlin, Germany).

In the assay, typically 11 different concentrations of each substance (0.1 nM, 0.33 nM, 1.1 nM, 3.8 nM, 13 nM, 44 nM, 0.15 μM, 0.51 μM, 1.7 μM, 5.9 μM and 20 μM) were analysed as duplicates on the same microtitre plate. For this purpose, 100-fold concentrated solutions in DMSO were prepared by serial dilutions (1:3.4) of a 2 mM stock solution into a clear, 384-well microtitre plate (Greiner Bio-One, Frickenhausen, Germany). From this, 50 nl were transferred into a black test plate (Greiner Bio-One, Frickenhausen, Germany). The test was started by the addition of 2 μl of a 2.5-fold concentrated BRD4(2) solution (final concentration typically 100 nM in the 5 μl of reaction volume) in aqueous assay buffer [50 mM HEPES pH 7.5, 50 mM sodium chloride (NaCl); 50 mM potassium fluoride (KF); 0.25 mM CHAPS and 0.05% serum albumin (BSA)] to the substances in the test plate. This was followed by a 10-minute incubation step at 22° C. for the pre-equilibration of putative complexes between BRD4(2) and the substances. Subsequently, 3 μl of a 1.67-fold concentrated solution (in assay buffer) consisting of Ac-H4 peptide (83.5 nM) and TR-FRET detection reagents [83.5 nM anti-6His-XL665 (Cisbio Bioassays, Codolet, France) and 12.52 nM streptavidin-Eu (Perkin Elmer, # W1024)] were added to the assay buffer.

The mixture was then incubated in the dark at 22° C. for one hour and then at 4° C. for at least 3 hours and for no longer than overnight. The formation of BRD4(2)/Ac-H4 complexes was determined by the measurement of the resonance energy transfer from the streptavidin-Eu chelate to the anti-6His-XL665 antibody present in the reaction. For this purpose, the fluorescence emission was measured at 620 nm and 665 nm after excitation at 330-350 nm in a TR-FRET measuring instrument, for example a Rubystar or Pherastar (both from BMG Lab Technologies, Offenburg, Germany) or a Viewlux (Perkin-Elmer). The ratio of the emissions at 665 nm and at 622 nm was taken as an indicator of the amount of BRD4(2)/Ac-H4 complexes formed.

The data (ratios) obtained were normalized, with 0% inhibition corresponding to the mean from the measurements for a set of controls (typically 32 data points) in which all the reagents were present. In these, in place of test substances, 50 nl of DMSO (100%) were used Inhibition of 100% corresponded to the mean from the measurements for a set of controls (typically 32 data points) in which all the reagents except BRD4(2) were present. The IC₅₀ was determined by regression analysis based on a 4-parameter equation (minimum, maximum, IC₅₀, Hill; Y=max+(min−max)/(1+(X/IC₅₀)Hill)).

3. Cell Assay

Cell Proliferation Assay

In accordance with the invention, the ability of the substances to inhibit cell proliferation was determined Cell viability was determined by means of the alamarBlue® reagent (Invitrogen) in a Victor X3 Multilabel Reader (Perkin Elmer). The excitation wavelength was 530 nm and the emission wavelength 590 nM.

The MOLM-13 cells (DSMZ, ACC 554) were sown at a concentration of 4000 cells/well in 100 μl of growth medium (RPMI1640, 10% FCS) on 96-well microtitre plates.

The B16F10 cells (ATCC, CRL-6475) were sown at a concentration of 300-500 cells/well in 100 μl of growth medium (DMEM with phenol red, 10% FCS) on 96-well microtitre plates.

The MOLP-8 cells (DSMZ, ACC 569) were sown at a concentration of 4000 cells/well in 100 μl of growth medium (RPMI1640, 20% FCS) on 96-well microtitre plates.

After overnight incubation at 37° C., the fluorescence values were determined (CI values). Then the plates were treated with various substance dilutions (1E-5 M, 3E-6 M, 1E-6 M, 3E-7 M, 1E-7 M, 3E-8 M, 1E-8 M) and incubated at 37° C. over 96 hours (MOLM-13, B16F10 cells) or 120 hours (MOLP-8 cells). Subsequently, the fluorescence values were determined (CO values). For the data analysis, the CI values were subtracted from the CO values and the results were compared between cells which had been treated with various dilutions of the substance or only with buffer solution. The IC₅₀ values (substance concentration needed for 50% inhibition of cell proliferation) were calculated therefrom.

4. Results:

4.1 Binding Assay

Table 3 shows the results from the BRD4(1) binding assay.

TABLE 3

| | IC₅₀ [BRD4(1)] |
|---|---|
| Example | (nmol/l) |
| 1 | 173 |
| 2 | 144 |
| 3 | 63 |
| 4 | 50 |
| 5 | 399 |
| 6 | 74 |
| 7 | 167 |
| 8 | 98 |
| 9 | 249 |
| 10 | 308 |
| 11 | 239 |
| 12 | 156 |
| 13 | 168 |
| 14 | 283 |
| 15 | 128 |
| 16 | 257 |
| 17 | 114 |
| 18 | 190 |
| 19 | 148 |
| 20 | 105 |
| 21 | 108 |
| 22 | 175 |
| 23 | 176 |
| 24 | 143 |
| 25 | 454 |
| 26 | 374 |
| 27 | 230 |
| 28 | 366 |
| 29 | 185 |
| 30 | 384 |
| 31 | 192 |
| 32 | 371 |
| 33 | 310 |
| 34 | 511 |
| 35 | 365 |
| 36 | 482 |
| 37 | 422 |
| 38 | 272 |
| 39 | 1040 |
| 40 | 1280 |
| 41 | 183 |
| 42 | 120 |
| 43 | 103 |
| 44 | 55 |
| 45 | 264 |
| 46 | 145 |
| 47 | 95 |
| 48 | 237 |

Table 4 shows the results from the BRD4(2) binding assay.

TABLE 4

| | IC₅₀ [BRD4(2)] |
|---|---|
| Example | (nmol/l) |
| 1 | 326 |
| 2 | 222 |
| 3 | 85 |
| 5 | 487 |
| 6 | 80 |
| 7 | 159 |
| 8 | 171 |
| 9 | 62 |
| 10 | 64 |
| 11 | 107 |
| 12 | 47 |
| 13 | 51 |
| 14 | 78 |
| 15 | 51 |
| 16 | 97 |
| 17 | 83 |
| 18 | 111 |
| 19 | 110 |
| 20 | 43 |
| 21 | 54 |
| 22 | 86 |
| 23 | 152 |
| 24 | 102 |
| 25 | 357 |
| 26 | 391 |
| 27 | 419 |
| 28 | 513 |
| 29 | 158 |
| 30 | 351 |
| 31 | 126 |
| 32 | 482 |
| 33 | 383 |
| 34 | 238 |
| 35 | 217 |
| 36 | 441 |
| 37 | 410 |
| 38 | 382 |
| 39 | 262 |
| 40 | 386 |
| 41 | 48 |
| 42 | 68 |
| 43 | 54 |
| 44 | 73 |
| 45 | 83 |
| 46 | 148 |
| 47 | 134 |
| 48 | 112 |

4.2 Cell Proliferation Assay

The cell lines studied are representative of the following indications:

MOLM-13 human AML (acute myeloid leukaemia) cell line

B16F10 mouse melanoma cell line

MOLP-8 human multiple myeloma cell line

Table 5 shows the results from the MOLM-13 cell proliferation assay.

TABLE 5
The ability of the inventive compounds to inhibit the
proliferation of the MOLM-13 cell line was determined.

| | IC₅₀ [MOLM-13] |
|---|---|
| Example | (nmol/l) |
| 1 | 1160 |
| 2 | 811 |
| 3 | 289 |
| 4 | 356 |
| 5 | 2740 |
| 6 | 698 |
| 7 | 1440 |
| 8 | 627 |
| 9 | 366 |
| 10 | 527 |
| 11 | 364 |
| 12 | 300 |
| 13 | 223 |
| 14 | 233 |
| 15 | 90 |
| 16 | 374 |
| 17 | 315 |
| 18 | 257 |
| 19 | 335 |
| 20 | 351 |
| 21 | 275 |
| 22 | 261 |
| 23 | 1260 |
| 24 | 967 |
| 25 | 1360 |
| 26 | 1580 |
| 27 | 1950 |
| 28 | 2760 |
| 29 | 2270 |
| 30 | 1700 |
| 31 | 1790 |
| 32 | 3450 |
| 33 | 1050 |
| 34 | 2100 |
| 35 | 2480 |
| 36 | 1080 |
| 37 | 1080 |
| 38 | 1790 |
| 39 | 2850 |
| 40 | >10 000 |
| 41 | 145 |
| 42 | 149 |
| 43 | 145 |
| 44 | 340 |
| 45 | 1340 |
| 46 | 719 |
| 47 | 407 |
| 48 | 1250 |

Table 6 shows the results from the B16F10 cell proliferation assay.

TABLE 6
The ability of the inventive compounds to inhibit the
proliferation of the B16F10 cell line was determined.

| | IC₅₀ [B16F10] |
|---|---|
| Example | (nmol/l) |
| 1 | 1500 |
| 2 | 1690 |
| 3 | 327 |
| 4 | 643 |
| 6 | 371 |
| 7 | 1020 |
| 8 | 584 |
| 9 | 365 |
| 11 | 768 |
| 12 | 179 |
| 13 | 136 |
| 14 | 178 |
| 15 | 224 |
| 16 | 251 |
| 17 | 378 |
| 18 | 287 |
| 19 | 248 |
| 20 | 256 |
| 21 | 403 |
| 22 | 522 |
| 23 | 1960 |
| 24 | 732 |
| 26 | 4010 |
| 27 | 2490 |
| 28 | 2830 |
| 29 | 2710 |
| 30 | 2680 |
| 31 | 3240 |
| 32 | 6420 |
| 33 | 1750 |
| 34 | 3570 |
| 35 | 2400 |
| 36 | 2090 |
| 37 | 1510 |
| 38 | 1780 |
| 39 | 3280 |
| 40 | 4980 |
| 41 | 175 |
| 42 | 79 |
| 43 | 212 |
| 44 | 460 |
| 45 | 1570 |
| 46 | 600 |
| 47 | 423 |
| 48 | 1450 |

Table 7 shows the results from the MOLP-8 cell proliferation assay.

TABLE 7
The ability of the inventive compounds to inhibit the
proliferation of the MOLP-8 cell line was determined.

| | IC₅₀ [MOLP-8] |
|---|---|
| Example | (nmol/l) |
| 1 | 1470 |
| 2 | 727 |
| 3 | 301 |
| 4 | 468 |
| 6 | 271 |
| 7 | 637 |
| 8 | 521 |
| 9 | 139 |
| 10 | 260 |
| 11 | 102 |
| 12 | 98 |
| 13 | 98 |
| 14 | 115 |
| 15 | 109 |
| 16 | 96 |
| 17 | 69 |
| 18 | 148 |
| 19 | 124 |
| 20 | 120 |
| 21 | 69 |
| 22 | 93 |
| 23 | 1020 |
| 24 | 662 |
| 25 | 1360 |
| 26 | 1030 |
| 27 | 1130 |
| 28 | 1800 |
| 29 | 1120 |
| 30 | 869 |
| 31 | 1250 |
| 32 | 2580 |
| 33 | 509 |
| 34 | 1540 |
| 35 | 1390 |
| 36 | 675 |
| 37 | 479 |
| 38 | 758 |
| 39 | 1740 |
| 40 | 6620 |
| 41 | 77 |
| 42 | 61 |
| 43 | 35 |
| 44 | 101 |
| 45 | 1100 |
| 46 | 365 |
| 47 | 496 |
| 48 | 803 |

1. A compound of the general formula (I) 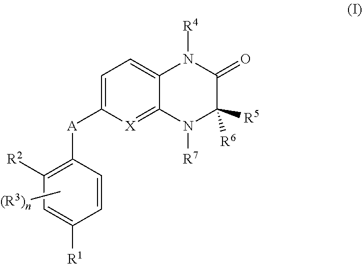
in which
A is —NH— or —O—,
X is —CH—,
n is 0 or 1,
R¹ is a —C(═O)NR⁸R⁹ or —S(═O)₂NR⁸R⁹ group,
R² is hydrogen, halogen, cyano, C₂-C₄-alkenyl, C₂-C₄-alkynyl, C₁-C₄-alkoxy, halo-C₁-C₄-alkoxy, C₁-C₄ alkylthio, halo-C₁-C₄-alkylthio, or —NR¹⁰R¹¹,
R³ is halogen, C₁-C₃-alkyl, C₁-C₃-alkoxy, trifluoromethyl or cyano and may be bonded to any of the still-unoccupied positions in the aromatic system,
R⁴ is methyl or ethyl,
R⁵ is hydrogen or C₁-C₃-alkyl,
R⁶ is hydrogen or C₁-C₃-alkyl, or
R⁵ and R⁶ together are C₂-C₅-alkylene,
R⁷ is C₁-C₆-alkyl, C₃-C₈-cycloalkyl, 4- to 8-membered heterocycloalkyl, phenyl or phenyl-C₁-C₃-alkyl in which each phenyl radical may optionally be mono-, di- or trisubstituted identically or differently by halogen, cyano, C₁-C₄-alkyl, C₂-C₄-alkenyl, C₂-C₄-alkynyl, C₁-C₄-alkoxy, halo-C₁-C₄-alkyl or halo-C₁-C₄-alkoxy,
R⁸ is C₁-C₆-alkyl which may optionally be mono-, di- or trisubstituted identically or differently by hydroxyl, oxo, fluorine, cyano, C₁-C₄-alkoxy, halo-C₁-C₄-alkoxy, —NR¹⁰R¹¹, 4- to 8-membered heterocycloalkyl, 4- to 8-membered heterocycloalkenyl, C₅-C₁₁-heterospirocycloalkyl, bridged C₆-C₁₂-heterocycloalkyl, C₆-C12-heterobicycloalkyl, phenyl or 5- to 6-membered heteroaryl,
in which 4- to 8-membered heterocycloalkyl, 4- to 8-membered heterocycloalkenyl, C₅-C₁₁-heterospirocycloalkyl, bridged C₆-C₁₂-heterocycloalkyl, C₆-C12-heterobicycloalkyl may optionally be monosubstituted by oxo or C₁-C₃-alkyl,
and in which phenyl and 5- to 6-membered heteroaryl may optionally be mono- or disubstituted identically or differently by halogen, cyano, trifluoromethyl, C₁-C₃-alkyl or C₁-C₃-alkoxy, or
is C₃-C₆-alkenyl or C₃-C₆-alkynyl, or
is C₃-C₈-cycloalkyl or C₄-C₈-cycloalkenyl, which may optionally be mono- or disubstituted identically or differently by hydroxyl, oxo, cyano, fluorine, C₁-C₃-alkyl, C₁-C₃-alkoxy, trifluoromethyl or —NR¹⁰R¹¹, or
is 4- to 8-membered heterocycloalkyl, 4- to 8-membered heterocycloalkenyl, C₅-C₁₁-heterospirocycloalkyl, bridged C₆-C₁₂-heterocycloalkyl, or C₆-C₁₂-heterobicycloalkyl, which may optionally be mono- or disubstituted identically or differently by hydroxyl, oxo, cyano, fluorine, C₁-C₃-alkyl, C₁-C₃-alkoxy, trifluoromethyl, C₁-C₃-alkylcarbonyl or —NR¹⁰R¹¹,
R⁹ is hydrogen or C₁-C₃-alkyl, or
R⁸ and R⁹, together with the nitrogen atom to which they are bonded, are 4- to 8-membered heterocycloalkyl, 4- to 8-membered heterocycloalkenyl, C₅-C₁₁-heterospirocycloalkyl, bridged C₆-C₁₂-heterocycloalkyl or C₆-C₁₂-heterobicycloalkyl, which may optionally be mono- or disubstituted identically or differently by hydroxyl, oxo, cyano, fluorine, C₁-C₃-alkyl, C₁-C₃-alkoxy, trifluoromethyl or —NR¹⁰R¹¹,
R¹⁰ and R¹¹ are each independently hydrogen or C₁-C₃-alkyl optionally mono- or disubstituted identically or differently by hydroxyl, oxo or fluorine, or
R¹⁰ and R¹¹, together with the nitrogen atom to which they are bonded, are 4- to 8-membered heterocycloalkyl which may optionally be mono- or disubstituted identically or differently by hydroxyl, oxo, cyano, fluorine, C₃-C₆-cycloalkyl-C₁-C₃-alkyl or C₁-C₃-alkyl,
R¹² is C₁-C₆-alkyl or phenyl-C₁-C₃-alkyl,
or a diastereomer or physiologically acceptable salt thereof,
excluding the compounds
4-{[(3R)-4-cyclopentyl-3-ethyl-1-methyl-2-oxo-1,2,3,4-tetrahydroquinoxalin-6-yl]amino}-3-methoxy-N-[2-methyl-1-(pyrrolidin-1-yl)propan-2-yl]benzamide and
4-{[(3R)-4-cyclohexyl-1,3-dimethyl-2-oxo-1,2,3,4-tetrahydroquinoxalin-6-yl]amino}-N-[1-(dimethylamino)-2-methylpropan-2-yl]-3-methoxybenzamide.

2. A compound according to claim 1, in which
A is —NH— or —O—,
X is —CH—,
n is 0 or 1,
R¹ is a —C(═O)NR⁸R⁹ or —S(═O)₂NR⁸R⁹ group,
R² is hydrogen, fluorine, chlorine, cyano, C₁-C₃-alkyl, fluoro-C₁-C₃-alkyl, C₁-C₃-alkoxy, fluoro-C₁-C₃-alkoxy, C₁-C₃-alkylthio or fluoro-C₁-C₃-alkylthio,
R³ is fluorine, chlorine or cyano and may be bonded to any of the still-unoccupied positions in the aromatic system,
R⁴ is methyl or ethyl,
R⁵ is C₁-C₃-alkyl,
R⁶ is hydrogen,
R⁷ is C₂-C₅-alkyl, C₃-C₇-cycloalkyl, 4- to 7-membered heterocycloalkyl or phenyl-C₁-C₃-alkyl,
in which the phenyl radical may optionally be mono- or disubstituted identically or differently by fluorine, chlorine, bromine, cyano, C₁-C₃-alkyl, C₁-C₃-alkoxy, or trifluoromethyl,
R⁸ is C₁-C₆-alkyl which may optionally be mono-, di- or trisubstituted identically or differently by hydroxyl, oxo, fluorine, cyano, C₁-C₃-alkoxy, fluoro-C₁-C₃-alkoxy, —NR¹⁰R¹¹, 4- to 8-membered heterocycloalkyl, phenyl or 5- to 6-membered heteroaryl,
in which the 4- to 8-membered heterocycloalkyl may optionally be monosubstituted by oxo or C₁-C₃-alkyl, or
is C₃-C₈-cycloalkyl which may optionally be mono- or disubstituted identically or differently by hydroxyl, oxo, cyano, fluorine or —NR¹⁰R¹¹, or
is 4- to 8-membered heterocycloalkyl, C₆-C₈-heterospirocycloalkyl, bridged C₆-C₁₀-heterocycloalkyl or C₆-C₁₀-heterobicycloalkyl, which may optionally be mono- or disubstituted identically or differently by hydroxyl, oxo, cyano, fluorine, C₁-C₃-alkyl, C₁-C₃-alkylcarbonyl or —NR¹⁰R¹¹,
R⁹ is hydrogen or C₁-C₃-alkyl, or
R⁸ and R⁹, together with the nitrogen atom to which they are bonded, are 4- to 8-membered heterocycloalkyl, C₆-C₈-heterospirocycloalkyl, bridged C₆-C₁₀-heterocycloalkyl or C₆-C₁₀-heterobicycloalkyl, which may optionally be mono- or disubstituted identically or differently by hydroxyl, oxo or C₁-C₃-alkyl,
R¹⁰ and R¹¹ are each independently hydrogen or optionally mono-hydroxyl-, -oxo- or -fluorine-substituted C₁-C₃-alkyl, or
R¹⁰ and R¹¹, together with the nitrogen atom to which they are bonded, are 4- to 7-membered heterocycloalkyl which may optionally be mono- or disubstituted identically or differently by hydroxyl, cyano, fluorine, cyclopropylmethyl or C₁-C₃-alkyl,
or a diastereomer or physiologically acceptable salt thereof,
excluding the compounds
4-{[(3R)-4-cyclopentyl-3-ethyl-1-methyl-2-oxo-1,2,3,4-tetrahydroquinoxalin-6-yl]amino}-3-methoxy-N-[2-methyl-1-(pyrrolidin-1-yl)propan-2-yl]benzamide and
4-{[(3R)-4-cyclohexyl-1,3-dimethyl-2-oxo-1,2,3,4-tetrahydroquinoxalin-6-yl]amino}-N-[1-(dimethylamino)-2-methylpropan-2-yl]-3-methoxybenzamide.

3. A compound according to claim 1, in which
A is —NH— or —O—,
X is —CH—,
n is 0,
R¹ is a —C(═O)NR⁸R⁹ or —S(═O)₂NR⁸R⁹ group,
R² is hydrogen, fluorine, chlorine, cyano, C₁-C₃-alkyl, fluoro-C₁-C₃-alkyl, C₁-C₃-alkoxy, fluoro-C₁-C₃-alkoxy, C₁-C₃-alkylthio or fluoro-C₁-C₃-alkylthio,
R⁴ is methyl,
R⁵ is methyl or ethyl,
R⁶ is hydrogen,
R⁷ is C₃-C₅-alkyl, C₃-C₇-cycloalkyl, 4- to 7-membered heterocycloalkyl or phenyl-C₁-C₃-alkyl,
in which the phenyl radical may optionally be mono- or disubstituted identically or differently by fluorine, C₁-C₃-alkyl or C₁-C₃-alkoxy,
R⁸ is C₁-C₄-alkyl which may optionally be monosubstituted by —NR¹⁰R¹¹ or 4- to 8-membered heterocycloalkyl,
in which the 4- to 8-membered heterocycloalkyl may optionally be monosubstituted by oxo or C₁-C₃-alkyl, or
is C₃-C₈-cycloalkyl which may optionally be monosubstituted by oxo or —NR¹⁰R¹¹, or
is 4- to 8-membered heterocycloalkyl which may optionally be monosubstituted by oxo, C₁-C₃-alkyl or C₁-C₃-alkylcarbonyl,
R⁹ is hydrogen or methyl, or
R⁸ and R⁹, together with the nitrogen atom to which they are bonded, are 4- to 8-membered heterocycloalkyl or C₆-C₈-heterospirocycloalkyl, which may optionally be mono- or disubstituted identically or differently by oxo or C₁-C₃-alkyl,
R¹⁰ and R¹¹ are each independently hydrogen, methyl or ethyl, or
R¹⁰ and R¹¹, together with the nitrogen atom to which they are bonded, are 4- to 7-membered heterocycloalkyl which may optionally be mono- or disubstituted identically or differently by fluorine, cyclopropylmethyl or C₁-C₃-alkyl,
or a diastereomer or physiologically acceptable salt thereof,
excluding the compounds
4-{[(3R)-4-cyclopentyl-3-ethyl-1-methyl-2-oxo-1,2,3,4-tetrahydroquinoxalin-6-yl]amino}-3-methoxy-N-[2-methyl-1-(pyrrolidin-1-yl)propan-2-yl]benzamide and
4-{[(3R)-4-cyclohexyl-1,3-dimethyl-2-oxo-1,2,3,4-tetrahydroquinoxalin-6-yl]amino}-N-[1-(dimethylamino)-2-methylpropan-2-yl]-3-methoxybenzamide.

4. A compound according to claim 1, in which
A is —NH— or —O—,
X is —CH—,
n is 0,
R¹ is a —C(═O)NR⁸R⁹ or —S(═O)₂NR⁸R⁹ group,
R² is hydrogen or methoxy,
R⁴ is methyl,
R⁵ is methyl,
R⁶ is hydrogen,
R⁷ is iso-propyl, C₅-C₇-cycloalkyl, 5- or 6-membered heterocycloalkyl or benzyl, in which the phenyl radical present in benzyl may optionally be mono- or disubstituted identically or differently by fluorine or methoxy,
R⁸ is C₁-C₂-alkyl which may optionally be monosubstituted by oxetanyl, pyrrolidinyl, piperidinyl, morpholinyl or piperazinyl,
in which piperazinyl may optionally be monosubstituted by C₁-C₃-alkyl, or
is C₃-C₆-cycloalkyl which may optionally be monosubstituted by oxo or —NR¹⁰R¹¹, or
is 4- to 6-membered heterocycloalkyl which may optionally be monosubstituted by oxo, methyl or acetyl,
R⁹ is hydrogen or methyl, or
R⁸ and R⁹, together with the nitrogen atom to which they are bonded, are 5- or 6-membered heterocycloalkyl or C₆-C₈-heterospirocycloalkyl, which may optionally be mono- or disubstituted identically or differently by oxo or C₁-C₃-alkyl,
R¹⁰ and R¹¹ are each independently hydrogen, methyl or ethyl, or
R¹⁰ and R¹¹, together with the nitrogen atom to which they are bonded, are pyrrolidinyl, piperidinyl, morpholinyl or piperazinyl bonded via the common nitrogen, where the piperazinyl may optionally be monosubstituted by cyclopropylmethyl or C₁-C₃-alkyl,
or a diastereomer or physiologically acceptable salt thereof.

5. A compound according to claim 1, in which
A is —NH— or —O—,
X is —CH—,
n is 0,
R¹ is a —C(═O)NR⁸R⁹ or —S(═O)₂NR⁸R⁹ group,
R² is hydrogen or methoxy,
R⁴ is methyl,
R⁵ is methyl,
R⁶ is hydrogen,
R⁷ is cyclopentyl, cycloheptyl, tetrahydropyran-4-yl, benzyl, 4-methoxybenzyl or 2,6-difluorobenzyl,
R⁸ is one of the following groups: 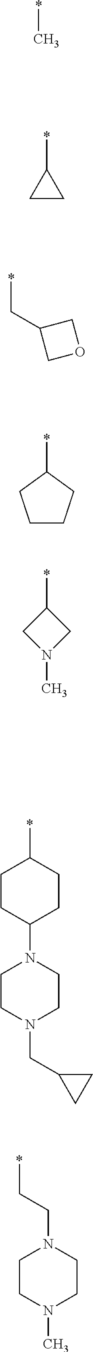 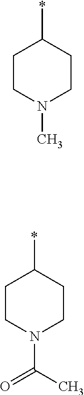
and in which “*” indicates the connection point to the nitrogen atom in —C(═O)NR⁸R⁹ or —S(═O)₂NR⁸R⁹
R⁹ is hydrogen or methyl, or
R⁸ and R⁹ together with the nitrogen atom to which they are bonded are one of the following groups: 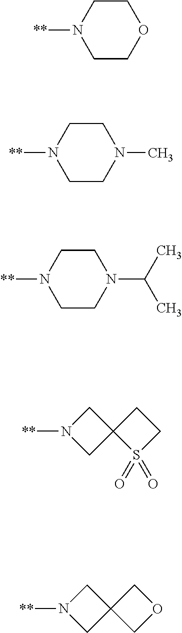
and in which “**” indicates the connection point to the carbonyl or sulphonyl group present in R′,
or a diastereomer or physiologically acceptable salt thereof.

6. A compound according to claim 1, in which
A is —NH—,
X is —CH—,
n is 0,
R¹ is a —C(═O)NR⁸R⁹ or —S(═O)₂NR⁸R⁹ group,
R² is hydrogen or methoxy,
R⁴ is methyl,
R⁵ is methyl,
R⁶ is hydrogen,
R⁷ is cyclopentyl, cycloheptyl, tetrahydropyran-4-yl, benzyl, 4-methoxybenzyl or 2,6-difluorobenzyl,
R⁸ is one of the following groups: 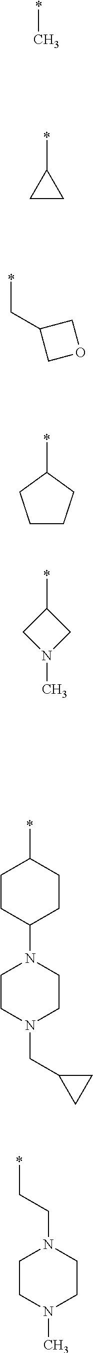 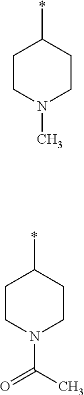
and in which “*” indicates the connection point to the nitrogen atom in —C(═O)NR⁸R⁹ or —S(═O)₂NR⁸R⁹
R⁹ is hydrogen or methyl, or
R⁸ and R⁹ together with the nitrogen atom to which they are bonded are one of the following groups: 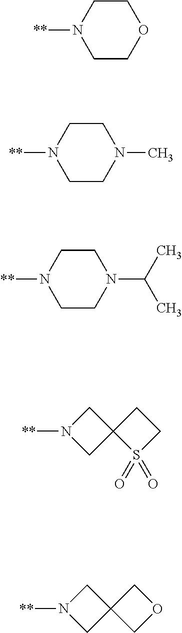
and in which “**” indicates the connection point to the carbonyl or sulphonyl group present in R′,
or a diastereomer or physiologically acceptable salt thereof.

7. A compound according to claim 1, in which
A is —O—,
X is —CH—,
n is 0,
R¹ is a —C(═O)NR⁸R⁹ or —S(═O)₂NR⁸R⁹ group,
R² is hydrogen or methoxy,
R⁴ is methyl,
R⁵ is methyl,
R⁶ is hydrogen,
R⁷ is cyclopentyl, cycloheptyl, tetrahydropyran-4-yl, benzyl, 4-methoxybenzyl or 2,6-difluorobenzyl,
R⁸ is one of the following groups: 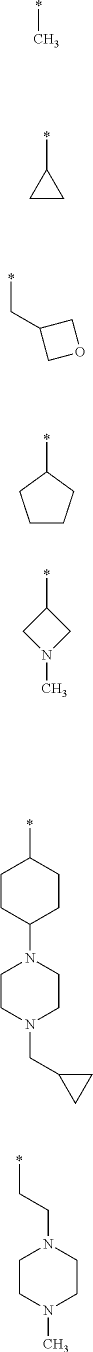 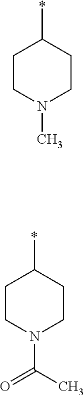
and in which “*” indicates the connection point to the nitrogen atom in —C(═O)NR⁸R⁹ or —S(═O)₂NR⁸R⁹
R⁹ is hydrogen or methyl, or
R⁸ and R⁹ together with the nitrogen atom to which they are bonded are one of the following groups: 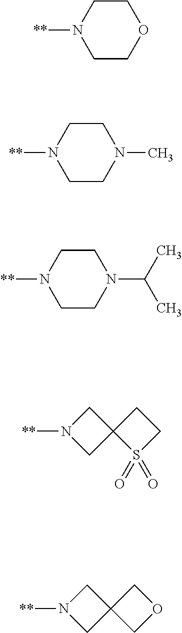
and in which “**” indicates the connection point to the carbonyl or sulphonyl group present in R′,
or a diastereomer or physiologically acceptable salt thereof.

8. A compound according to claim 1 selected from:
N-cyclopentyl-4-{[(3R)-4-cyclopentyl-1,3-dimethyl-2-oxo-1,2,3,4-tetrahydroquinoxalin-6-yl]amino}benzamide;
4-{[(3R)-4-cyclopentyl-1,3-dimethyl-2-oxo-1,2,3,4-tetrahydroquinoxalin-6-yl]amino}-N-cyclopropylbenzamide;
4-{[(3R)-4-cyclopentyl-1,3-dimethyl-2-oxo-1,2,3,4-tetrahydroquinoxalin-6-yl]amino}-N,N-dimethylbenzenesulphonamide;
4-{[(3R)-4-cyclopentyl-1,3-dimethyl-2-oxo-1,2,3,4-tetrahydroquinoxalin-6-yl]amino}-N-(1-methylpiperidin-4-yl)benzamide;
4-{[(3R)-4-cyclopentyl-1,3-dimethyl-2-oxo-1,2,3,4-tetrahydroquinoxalin-6-yl]oxy}-N-cyclopropylbenzamide;
(3R)-4-cyclopentyl-1,3-dimethyl-6-{[4-(morpholin-4-ylcarbonyl)phenyl]amino}-3,4-dihydroquinoxalin-2(1H)-one;
4-{[(3R)-4-cyclopentyl-1,3-dimethyl-2-oxo-1,2,3,4-tetrahydroquinoxalin-6-yl]amino}-N-isopropylbenzamide;
4-{[(3R)-4-cyclopentyl-1,3-dimethyl-2-oxo-1,2,3,4-tetrahydroquinoxalin-6-yl]amino}-N,N-dimethylbenzamide;
4-{[(3R)-4-benzyl-1,3-dimethyl-2-oxo-1,2,3,4-tetrahydroquinoxalin-6-yl]amino}-N-(oxetan-3-ylmethyl)benzamide;
4-{[(3R)-4-benzyl-1,3-dimethyl-2-oxo-1,2,3,4-tetrahydroquinoxalin-6-yl]amino}-N-cyclopropylbenzamide;
4-{[(3R)-4-benzyl-1,3-dimethyl-2-oxo-1,2,3,4-tetrahydroquinoxalin-6-yl]amino}-N-(1-methylpiperidin-4-yl)benzamide;
(3R)-4-benzyl-1,3-dimethyl-6-{[4-(morpholin-4-ylcarbonyl)phenyl]amino}-3,4-dihydroquinoxalin-2(1H)-one;
(3R)-4-benzyl-1,3-dimethyl-6-{[4-(morpholin-4-ylsulphonyl)phenyl]amino}-3,4-dihydroquinoxalin-2(1H)-one;
4-{[(3R)-4-benzyl-1,3-dimethyl-2-oxo-1,2,3,4-tetrahydroquinoxalin-6-yl]amino}-N,N-dimethylbenzenesulphonamide;
(3R)-4-benzyl-1,3-dimethyl-6-({4-[(4-methylpiperazin-1-yl)sulphonyl]phenyl}amino)-3,4-dihydroquinoxalin-2(1H)-one;
(3R)-4-benzyl-6-({4-[(1,1-dioxido-1-thia-6-azaspiro[3.3]hept-6-yl)carbonyl]phenyl}amino)-1,3-dimethyl-3,4-dihydroquinoxalin-2(1H)-one;
4-{[(3R)-4-(4-methoxybenzyl)-1,3-dimethyl-2-oxo-1,2,3,4-tetrahydroquinoxalin-6-yl]amino}-N-(1-methylpiperidin-4-yl)benzamide;
(3R)-4-(4-methoxybenzyl)-1,3-dimethyl-6-{[4-(morpholin-4-ylcarbonyl)phenyl]amino}-3,4-dihydroquinoxalin-2(1H)-one;
4-{[(3R)-4-(4-methoxybenzyl)-1,3-dimethyl-2-oxo-1,2,3,4-tetrahydroquinoxalin-6-yl]amino}-N-(oxetan-3-ylmethyl)benzamide;
(3R)-4-(4-methoxybenzyl)-1,3-dimethyl-6-{[4-(2-oxa-6-azaspiro[3.3]hept-6-ylcarbonyl)phenyl]amino}-3,4-dihydroquinoxalin-2(1H)-one;
4-{[(3R)-4-(4-methoxybenzyl)-1,3-dimethyl-2-oxo-1,2,3,4-tetrahydroquinoxalin-6-yl]amino}-N-[2-(4-methylpiperazin-1-yl)ethyl]benzamide;
(3R)-4-(4-methoxybenzyl)-1,3-dimethyl-6-[(4-{[4-(propan-2-yl)piperazin-1-yl]carbonyl}phenyl)amino]-3,4-dihydroquinoxalin-2(1H)-one;
4-{[(3R)-4-cycloheptyl-1,3-dimethyl-2-oxo-1,2,3,4-tetrahydroquinoxalin-6-yl]amino}-N-(1-methylpiperidin-4-yl)benzamide;
(3R)-4-cycloheptyl-1,3-dimethyl-6-{[4-(morpholin-4-ylcarbonyl)phenyl]amino}-3,4-dihydroquinoxalin-2(1H)-one;
4-{[(3R)-4-cycloheptyl-1,3-dimethyl-2-oxo-1,2,3,4-tetrahydroquinoxalin-6-yl]amino}-N-(oxetan-3-ylmethyl)benzamide;
4-{[(3R)-1,3-dimethyl-2-oxo-4-(tetrahydro-2H-pyran-4-yl)-1,2,3,4-tetrahydroquinoxalin-6-yl]amino}-N-(1-methylpiperidin-4-yl)benzamide;
(3R)-1,3-dimethyl-6-{[4-(morpholin-4-ylcarbonyl)phenyl]amino}-4-(tetrahydro-2H-pyran-4-yl)-3,4-dihydroquinoxalin-2(1H)-one;
4-{[(3R)-1,3-dimethyl-2-oxo-4-(tetrahydro-2H-pyran-4-yl)-1,2,3,4-tetrahydroquinoxalin-6-yl]amino}-N-(oxetan-3-ylmethyl)benzamide;
(3R)-1,3-dimethyl-6-{[4-(2-oxa-6-azaspiro[3.3]hept-6-ylcarbonyl)phenyl]amino}-4-(tetrahydro-2H-pyran-4-yl)-3,4-dihydroquinoxalin-2(1H)-one;
4-{[(3R)-1,3-dimethyl-2-oxo-4-(tetrahydro-2H-pyran-4-yl)-1,2,3,4-tetrahydroquinoxalin-6-yl]amino}-N-[2-(4-methylpiperazin-1-yl)ethyl]benzamide;
(3R)-6-({4-[(1,1-dioxido-1-thia-6-azaspiro[3.3]hept-6-yl)carbonyl]phenyl}amino)-1,3-dimethyl-4-(tetrahydro-2H-pyran-4-yl)-3,4-dihydroquinoxalin-2(1H)-one;
N-(1-acetylpiperidin-4-yl)-4-{[(3R)-1,3-dimethyl-2-oxo-4-(tetrahydro-2H-pyran-4-yl)-1,2,3,4-tetrahydroquinoxalin-6-yl]amino}benzamide;
(3R)-1,3-dimethyl-6-[(4-{[4-(propan-2-yl)piperazin-1-yl]carbonyl}phenyl)amino]-4-(tetrahydro-2H-pyran-4-yl)-3,4-dihydroquinoxalin-2(1H)-one;
4-{[(3R)-1,3-dimethyl-2-oxo-4-(tetrahydro-2H-pyran-4-yl)-1,2,3,4-tetrahydroquinoxalin-6-yl]amino}-N-(1-methylazetidin-3-yl)benzamide;
N-cyclopropyl-4-{[(3R)-1,3-dimethyl-2-oxo-4-(tetrahydro-2H-pyran-4-yl)-1,2,3,4-tetrahydroquinoxalin-6-yl]amino}benzamide;
4-{[(3R)-1,3-dimethyl-2-oxo-4-(tetrahydro-2H-pyran-4-yl)-1,2,3,4-tetrahydroquinoxalin-6-yl]amino}-3-methoxy-N-(1-methylpiperidin-4-yl)benzamide;
N-{4-[4-(cyclopropylmethyl)piperazin-1-yl]cyclohexyl}-4-{[(3R)-1,3-dimethyl-2-oxo-4-(tetrahydro-2H-pyran-4-yl)-1,2,3,4-tetrahydroquinoxalin-6-yl]amino}-3-methoxybenzamide;
(3R)-6-({2-methoxy-4-[(4-methylpiperazin-1-yl)carbonyl]phenyl}amino)-1,3-dimethyl-4-(tetrahydro-2H-pyran-4-yl)-3,4-dihydroquinoxalin-2(1H)-one;
4-{[4-(2,6-difluorobenzyl)-1,3-dimethyl-2-oxo-1,2,3,4-tetrahydroquinoxalin-6-yl]amino}-N-(1-methylpiperidin-4-yl)benzamide;
N-{4-[4-(cyclopropylmethyl)piperazin-1-yl]cyclohexyl}-4-{[4-(2,6-difluorobenzyl)-1,3-dimethyl-2-oxo-1,2,3,4-tetrahydroquinoxalin-6-yl]amino}benzamide;
4-{[(3R)-4-(4-methoxybenzyl)-1,3-dimethyl-2-oxo-1,2,3,4-tetrahydroquinoxalin-6-yl]amino}-N,N-dimethylbenzenesulphonamide;
(3R)-4-(4-methoxybenzyl)-1,3-dimethyl-6-{[4-(morpholin-4-ylsulphonyl)phenyl]amino}-3,4-dihydroquinoxalin-2(1H)-one;
(3R)-4-(4-methoxybenzyl)-1,3-dimethyl-6-({4-[(4-methylpiperazin-1-yl)sulphonyl]phenyl}amino)-3,4-dihydroquinoxalin-2(1H)-one;
(3R)-4-(4-methoxybenzyl)-1,3-dimethyl-6-[(4-{[4-(propan-2-yl)piperazin-1-yl]sulphonyl}phenyl)amino]-3,4-dihydroquinoxalin-2(1H)-one;
4-{[(3R)-4-cycloheptyl-1,3-dimethyl-2-oxo-1,2,3,4-tetrahydroquinoxalin-6-yl]amino}-N,N-dimethylbenzenesulphonamide;
4-{[(3R)-1,3-dimethyl-2-oxo-4-(tetrahydro-2H-pyran-4-yl)-1,2,3,4-tetrahydroquinoxalin-6-yl]amino}-N,N-dimethylbenzenesulphonamide;
(3R)-1,3-dimethyl-6-{[4-(morpholin-4-ylsulphonyl)phenyl]amino}-4-(tetrahydro-2H-pyran-4-yl)-3,4-dihydroquinoxalin-2(1H)-one; and
(3R)-1,3-dimethyl-6-({4-[(4-methylpiperazin-1-yl)sulphonyl]phenyl}amino)-4-(tetrahydro-2H-pyran-4-yl)-3,4-dihydroquinoxalin-2(1H)-one,
or a diastereomer or physiologically acceptable salt thereof.

9. (canceled)

10. (canceled)

11. (canceled)

12. A method for the treatment of a neoplastic disorder comprising administering to a patient in need thereof a therapeutically effective amount of a compound according to claim 1.

13. A method for the treatment of melanoma, multiple myeloma and or acute myeloid leukaemia comprising administering to a patient in need thereof a therapeutically effective amount of a compound according to claim 1.

14. A method for the treatment of a hyperproliferative disorder comprising administering to a patient in need thereof a therapeutically effective amount of a compound according to claim 1.

15. A method for the treatment of a viral infection, neurodegenerative disorder, inflammation disorder, atherosclerotic disorder or male fertility control comprising administering to a patient in need thereof a therapeutically effective amount of a compound according to claim 1.

16. (canceled)

17. A pharmaceutical composition comprising a compound according to claim 1 in combination with one or more further pharmacologically active substances.

18. (canceled)

19. (canceled)

20. (canceled)

21. (canceled)

22. A compound of the general formula (XI) 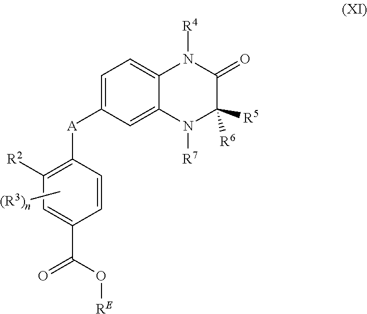
in which
A is —NH— or —O—,
R² is hydrogen, halogen, cyano, C₁-C₄-alkyl, C₂-C₄-alkenyl, C₂-C₄-alkynyl, halo-C₁-C₄-alkyl, C₁-C₄-alkoxy, halo-C₁-C₄-alkoxy, C₁-C₄-alkylthio, halo-C₁-C₄-alkylthio, or —NR¹⁰R¹¹,
R³ is halogen, C₁-C₃-alkyl, C₁-C₃-alkoxy, trifluoromethyl or cyano and may be bonded to any of the still-unoccupied positions in the aromatic system,
R⁴ is methyl or ethyl,
R⁵ is hydrogen or C₁-C₃-alkyl,
R⁶ is hydrogen or C₁-C₃-alkyl, or
R⁵ and R⁶ together are C₂-C₅-alkylene,
R⁷ is C₁-C₆-alkyl, C₃-C₈-cycloalkyl, 4- to 8-membered heterocycloalkyl, phenyl or phenyl-C₁-C₃-alkyl in which each phenyl radical may optionally be mono-, di- or trisubstituted identically or differently by halogen, cyano, C₁-C₄-alkyl, C₂-C₄-alkenyl, C₂-C₄-alkynyl, C₁-C₄-alkoxy, halo-C₁-C₄-alkyl or halo-C₁-C₄-alkoxy,
R¹⁰ and R¹¹ are each independently hydrogen or C₁-C₃-alkyl optionally mono- or disubstituted identically or differently by hydroxyl, oxo or fluorine, or
R¹⁰ and R¹¹, together with the nitrogen atom to which they are bonded, are 4- to 8-membered heterocycloalkyl which may optionally be mono- or disubstituted identically or differently by hydroxyl, oxo, cyano, fluorine, C₃-C₆-cycloalkyl-C₁-C₃-alkyl or C₁-C₃-alkyl,
n is 0 or 1,
and R^{E} is C₁-C₆-alkyl.

23. A compound of the general formula (XII) 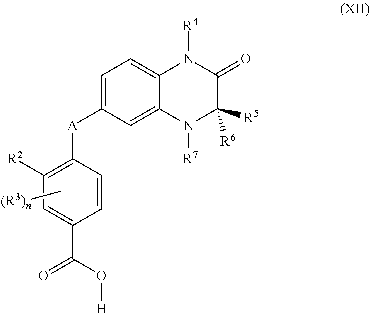
in which
A is —NH— or —O—,
R² is hydrogen, halogen, cyano, C₁-C₄-alkyl, C₂-C₄-alkenyl, C₂-C₄-alkynyl, halo-C₁-C₄-alkyl, C₁-C₄-alkoxy, halo-C₁-C₄-alkoxy, C₁-C₄-alkylthio, halo-C₁-C₄-alkylthio, or —NR¹⁰R¹¹,
R³ is halogen, C₁-C₃-alkyl, C₁-C₃-alkoxy, trifluoromethyl or cyano and may be bonded to any of the still-unoccupied positions in the aromatic system,
R⁴ is methyl or ethyl,
R⁵ is hydrogen or C₁-C₃-alkyl,
R⁶ is hydrogen or C₁-C₃-alkyl, or
R⁵ and R⁶ together are C₂-C₅-alkylene,
R⁷ is C₁-C₆-alkyl, C₃-C₈-cycloalkyl, 4- to 8-membered heterocycloalkyl, phenyl or phenyl-C₁-C₃-alkyl in which each phenyl radical may optionally be mono-, di- or trisubstituted identically or differently by halogen, cyano, C₁-C₄-alkyl, C₂-C₄-alkenyl, C₂-C₄-alkenyl, C₁-C₄-alkoxy, halo-C₁-C₄-alkyl or halo-C₁-C₄-alkoxy,
R¹⁰ and R¹¹ are each independently hydrogen or C₁-C₃-alkyl optionally mono- or disubstituted identically or differently by hydroxyl, oxo or fluorine, or
R¹⁰ and R¹¹, together with the nitrogen atom to which they are bonded, are 4- to 8-membered heterocycloalkyl which may optionally be mono- or disubstituted identically or differently by hydroxyl, oxo, cyano, fluorine, C₃-C₆-cycloalkyl-C₁-C₃-alkyl or C₁-C₃-alkyl,
n is 0 or 1.

24. A compound according to claim 22 selected from:
4-{[(3R)-4-cyclopentyl-1,3-dimethyl-2-oxo-1,2,3,4-tetrahydroquinoxalin-6-yl]amino}benzoic acid methyl ester;
4-{[(3R)-4-benzyl-1,3-dimethyl-2-oxo-1,2,3,4-tetrahydroquinoxalin-6-yl]amino}benzoic acid methyl ester;
4-{[(3R)-4-cyclopentyl-1,3-dimethyl-2-oxo-1,2,3,4-tetrahydroquinoxalin-6-yl]oxy}benzoic acid ethyl ester;
4-{[(3R)-4-(4-methoxybenzyl)-1,3-dimethyl-2-oxo-1,2,3,4-tetrahydroquinoxalin-6-yl]amino}benzoic acid methyl ester;
4-{[(3R)-4-cycloheptyl-1,3-dimethyl-2-oxo-1,2,3,4-tetrahydroquinoxalin-6-yl]amino}benzoic acid methyl ester;
4-{[(3R)-1,3-dimethyl-2-oxo-4-(tetrahydro-2H-pyran-4-yl)-1,2,3,4-tetrahydroquinoxalin-6-yl]amino}benzoic acid methyl ester;
4-{[(3R)-1,3-dimethyl-2-oxo-4-(tetrahydro-2H-pyran-4-yl)-1,2,3,4-tetrahydroquinoxalin-6-yl]amino}-3-methoxybenzoic acid methyl ester; and
4-{[4-(2,6-difluorobenzyl)-1,3-dimethyl-2-oxo-1,2,3,4-tetrahydroquinoxalin-6-yl]amino}benzoic acid ethyl ester.

25. A compound according to claim 23 selected from:
4-{[(3R)-4-cyclopentyl-1,3-dimethyl-2-oxo-1,2,3,4-tetrahydroquinoxalin-6-yl]amino}benzoic acid;
4-{[(3R)-4-benzyl-1,3-dimethyl-2-oxo-1,2,3,4-tetrahydroquinoxalin-6-yl]amino}benzoic acid;
4-{[(3R)-4-cyclopentyl-1,3-dimethyl-2-oxo-1,2,3,4-tetrahydroquinoxalin-6-yl]oxy}benzoic acid;
4-{[(3R)-4-(4-methoxybenzyl)-1,3-dimethyl-2-oxo-1,2,3,4-tetrahydroquinoxalin-6-yl]amino}benzoic acid;
4-{[(3R)-4-cycloheptyl-1,3-dimethyl-2-oxo-1,2,3,4-tetrahydroquinoxalin-6-yl]amino}benzoic acid;
4-{[(3R)-1,3-dimethyl-2-oxo-4-(tetrahydro-2H-pyran-4-yl)-1,2,3,4-tetrahydroquinoxalin-6-yl]amino}benzoic acid;
4-{[(3R)-1,3-dimethyl-2-oxo-4-(tetrahydro-2H-pyran-4-yl)-1,2,3,4-tetrahydroquinoxalin-6-yl]amino}-3-methoxybenzoic acid; and
4-{[4-(2,6-difluorobenzyl)-1,3-dimethyl-2-oxo-1,2,3,4-tetrahydroquinoxalin-6-yl]amino}benzoic acid.
